SEQUENCE LISTING (80 sequences)

SEQ ID NO: 1 - protein, 411 aa, Homo sapiens
MRAHPGGGRCCPEQEEGESAAGGSGAGGDSAIEQGGQGSALAPSPVSGVRREGARGGGRGRGRWKQAGRGGGVCGRGRGRGRGRGRGRGRGRGRGRPPSGGSGLGGDGGGCGGGGSGGGGAPRREPVPFPSGSAGPGPRGPRATESGKRMDCPALPPGWKKEEVIRKSGLSAGKSDVYYFSPSGKKFRSKPQLARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEMDIEMDSGDEA

SEQ ID NO: 2 - DNA, 1233 bp, Homo sapiens
  CDS: (1)..(1236)
atgcgcgcgcacccggggggaggccgctgctgcccggagcaggaggagggggagagtgcggcgggcggcagcggcgctggcggcgactccgccatagagcaggggggccagggcagcgcgctcgccccgtccccggtgagcggcgtgcgcagggaaggcgctcggggcggcggccgtggccgggggcggtggaagcaggcgggccggggcggcggcgtctgtggccgtggccggggccggggccgtggccggggacggggacggggccggggccggggccgcggccgtcccccgagtggcggcagcggccttggcggcgacggcggcggctgcggcggcggcggcagcggtggcggcggcgccccccggcgggagccggtccctttcccgtcggggagcgcggggccggggcccaggggaccccgggccacggagagcgggaagaggatggattgcccggccctcccccccggatggaagaaggaggaagtgatccgaaaatctgggctaagtgctggcaagagcgatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctcaatcaaaataagggtaaaccagacttgaatacaacattgccaattagacaaacagcatcaattttcaaacaaccggtaaccaaagtcacaaatcatcctagtaataaagtgaaatcagacccacaacgaatgaatgaacagccacgtcagcttttctgggagaagaggctacaaggacttagtgcatcagatgtaacagaacaaattataaaaaccatggaactacccaaaggtcttcaaggagttggtccaggtagcaatgatgagacccttttatctgctgttgccagtgctttgcacacaagctctgcgccaatcacagggcaagtctccgctgctgtggaaaagaaccctgctgtttggcttaacacatctcaacccctctgcaaagcttttattgtcacagatgaagacatcaggaaacaggaagagcgagtacagcaagtacgcaagaaattggaagaagcactgatggcagacatcttgtcgcgagctgctgatacagaagagatggatattgaaatggacagtggagatgaagcc

SEQ ID NO: 3 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 4 - DNA, 588 bp, Homo sapiens
  CDS: (1)..(591)
atgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacggatgtgagg

SEQ ID NO: 5 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 6 - DNA, 723 bp, Homo sapiens
  CDS: (1)..(726)
atgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcc

SEQ ID NO: 7 - protein, 155 aa, Homo sapiens [sequence omitted]

SEQ ID NO: 8 - DNA, 465 bp, Homo sapiens
  CDS: (1)..(468)
atggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcattgaaacgaactgggcagtataaacttggatccaaaacaggacctgggcagaaagctatactttttcttccaatgtctgctaagagc

SEQ ID NO: 9 - protein, 324 aa, Homo sapiens
MFPSPALTPTPFSVKDILNLEQQQRSLAAAGELSARLEATLAPSSCMLAAFKPEAYAGPEAAAPGLPELRAELGRAPSPAKCASAFPAAPAFYPRAYSDPDPAKDPRAEKKELCALQKAVELEKTEADNAERPRARRRRKPRVLFSQAQVYELERRFKQQRYLSAPERDQLASVLKLTSTQVKIWFQNRRYKCKRQRQDQTLELVGLPPPPPPPARRIAVPVLVRDGKPCLGDSAPYAPAYGVGLNPYGYNAYPAYPGYGGAACSPGYSCTAAYPAGPSPAQPATAAANNNFVNFGVGDLNAVQSPGIPQSNSGVSTLHGIRAW

SEQ ID NO: 10 - DNA, 972 bp, Homo sapiens
  CDS: (1)..(975)
atgttccccagccctgctctcacgcccacgcccttctcagtcaaagacatcctaaacctggaacagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgagctggagcggcgcttcaagcagcagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctgg

SEQ ID NO: 11 - protein, 442 aa, Homo sapiens [sequence omitted]

SEQ ID NO: 12 - DNA, 1326 bp, Homo sapiens
  CDS: (1)..(1329)
atgtatcagagcttggccatggccgccaaccacgggccgccccccggtgcctaccaggcgggcggccccggccccttcatgcacggcgcgggcgccgcgtcctcgccagtctacctgcccacaccgcgggtgccctcctccgttctgggcctgtcctacctccagggcggaggcgcgggctctgcgtccggaggcccctcgggcggcagccccggtggggccgcgtctggtgcggggcccgggacccagcagggcagcccgggatggagccaggcgggagcgaccggagccgcttacaccccgccgccggtgtcgccgcgcttctccttcccggggaccaccgggtccctggcggcggcggcggcggctgccgccgcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggacgggtcactatctgtgcaacgcctgtggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtgcccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagtctggtcttggccgacagtcacggggacataatcactgcg

SEQ ID NO: 13 - protein, 507 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKGMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPQPPQPQPRQEMGRSPVDSLSSSSSSYDGSDREDPRGDFHSPIVLGRPPNTEDRESPSVKRMRMDAWVT

SEQ ID NO: 14 - DNA, 1521 bp, Homo sapiens
  CDS: (1)..(1524)
atggggcggaagaaaatacaaatcacacgcataatggatgaaaggaaccgacaggtcacttttacaaagagaaagtttggattaatgaagaaagcctatgaacttagtgtgctctgtgactgtgaaatagcactcatcattttcaacagctctaacaaactgtttcaatatgctagcactgatatggacaaagttcttctcaagtatacagaatataatgaacctcatgaaagcagaaccaactcggatattgttgaggctctgaacaagaaggaacacagagggtgcgacagcccagaccctgatacttcatatgtgctaactccacatacagaagaaaaatataaaaaaattaatgaggaatttgataatatgatgcggaatcataaaatcgcacctggtctgccacctcagaacttttcaatgtctgtcacagttccagtgaccagccccaatgctttgtcctacactaacccagggagttcactggtgtccccatctttggcagccagctcaacgttaacagattcaagcatgctctctccacctcaaaccacattacatagaaatgtgtctcctggagctcctcagagaccaccaagtactggcaatgcaggtgggatgttgagcactacagacctcacagtgccaaatggagctggaagcagtccagtggggaatggatttgtaaactcaagagcttctccaaatttgattggagctactggtgcaaatagcttaggcaaagtcatgcctacaaagtctccccctccaccaggtggtggtaatcttggaatgaacagtaggaaaccagatcttcgagttgtcatccccccttcaagcaagggcatgatgcctccactatcggaggaagaggaattggagttgaacacccaaaggatcagtagttctcaagccactcaacctcttgctaccccagtcgtgtctgtgacaaccccaagcttgcctccgcaaggacttgtgtactcagcaatgccgactgcctacaacactgattattcactgaccagcgctgacctgtcagcccttcaaggcttcaactcgccaggaatgctgtcgctgggacaggtgtcggcctggcagcagcaccacctaggacaagcagccctcagctctcttgttgctggagggcagttatctcagggttccaatttatccattaataccaaccaaaacatcagcatcaagtccgaaccgatttcacctcctcgggatcgtatgaccccatcgggcttccagcagcagcagcagcagcagcagcagcagcagccgccgccaccaccgcagccccagccacaacccccgcagccccagccccgacaggaaatggggcgctcccctgtggacagtctgagcagctctagtagctcctatgatggcagtgatcgggaggatccacggggcgacttccattctccaattgtgcttggccgacccccaaacactgaggacagagaaagcccttctgtaaagcgaatgaggatggacgcgtgggtgacc

SEQ ID NO: 15 - protein, 365 aa, Homo sapiens
MGRKKIQISRILDQRNRQVTFTKRKFGLMKKAYELSVLCDCEIALIIFNSANRLFQYASTDMDRVLLKYTEYSEPHESRTNTDILETLKRRGIGLDGPELEPDEGPEEPGEKFRRLAGEGGDPALPRPRLYPAAPAMPSPDVVYGALPPPGCDPSGLGEALPAQSRPSPFRPAAPKAGPPGLVHPLFSPSHLTSKTPPPLYLPTEGRRSDLPGGLAGPRGGLNTSRSLYSGLQNPCSTATPGPPLGSFPFLPGGPPVGAEAWARRVPQPAAPPRRPPQSASSLSASLRPPGAPATFLRPSPIPCSSPGPWQSLCGLGPPCAGCPWPTAGPGRRSPGGTSPERSPGTARARGDPTSLQASSEKTQQ

SEQ ID NO: 16 - DNA, 1095 bp, Homo sapiens
  CDS: (1)..(1098)
atggggaggaaaaaaatccagatctcccgcatcctggaccaaaggaatcggcaggtgacgttcaccaagcggaagttcgggctgatgaagaaggcctatgagctgagcgtgctctgtgactgtgagatagccctcatcatcttcaacagcgccaaccgcctcttccagtatgccagcacggacatggaccgtgtgctgctgaagtacacagagtacagcgagccccacgagagccgcaccaacactgacatcctcgagacgctgaagcggaggggcattggcctcgatgggccagagctggagccggatgaagggcctgaggagccaggagagaagtttcggaggctggcaggcgaagggggtgatccggccttgccccgaccccggctgtatcctgcagctcctgctatgcccagcccagatgtggtatacggggccttaccgccaccaggctgtgaccccagtgggcttggggaagcactgcccgcccagagccgcccatctcccttccgaccagcagcccccaaagccgggcccccaggcctggtgcaccctctcttctcaccaagccacctcaccagcaagacaccacccccactgtacctgccgacggaagggcggaggtcagacctgcctggtggcctggctgggccccgagggggactaaacacctccagaagcctctacagtggcctgcagaacccctgctccactgcaactcccggacccccactggggagcttccccttcctccccggaggccccccagtgggggccgaagcctgggcgaggagggtcccccaacccgcggcgcctccccgccgacccccccagtcagcatcaagtctgagcgcctctctccggcccccgggggccccggcgactttcctaagaccttcccctatcccttgctcctcgcccggtccctggcagagcctctgcggcctgggcccgccctgcgccggctgcccttggccgacggctggccccggtaggagatcacccggtggcaccagcccagagcgctcgccaggtacggcgagggcacgtggggaccccacctccctccaggcctcttcagagaagacccaacag

SEQ ID NO: 17 - protein, 465 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 18 - DNA, 1395 bp, Homo sapiens
  CDS: (1)..(1398)
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaaca

SEQ ID NO: 19 - protein, 521 aa, Homo sapiens
MGRKKIQIQRITDERNRQVTFTKRKFGLMKKAYELSVLCDCEIALIIFNHSNKLFQYASTDMDKVLLKYTEYNEPHESRTNADIIETLRKKGFNGCDSPEPDGEDSLEQSPLLEDKYRRASEELDGLFRRYGSTVPAPNFAMPVTVPVSNQSSLQFSNPSGSLVTPSLVTSSLTDPRLLSPQQPALQRNSVSPGLPQRPASAGAMLGGDLNSANGACPSPVGNGYVSARASPGLLPVANGNSLNKVIPAKSPPPPTHSTQLGAPSRKPDLRVITSQAGKGLMHHLTEDHLDLNNAQRLGVSQSTHSLTTPVVSVATPSLLSQGLPFSSMPTAYNTDYQLTSAELSSLPAFSSPGGLSLGNVTAWQQPQQPQQPQQPQPPQQQPPQPQQPQPQQPQQPQQPPQQQSHLVPVSLSNLIPGSPLPHVGAALTVTTHPHISIKSEPVSPSRERSPAPPPPAVFPAARPEPGDGLSSPAGGSYETGDRDDGRGDFGPTLGLLRPAPEPEAEGSAVKRMRLDTWTLK

SEQ ID NO: 20 - DNA, 1563 bp, Homo sapiens
  CDS: (1)..(1566)
atggggaggaaaaagattcagatccagcgaatcaccgacgagcggaaccgacaggtgactttcaccaagcggaagtttggcctgatgaagaaggcgtatgagctgagcgtgctatgtgactgcgagatcgcactcatcatcttcaaccactccaacaagctgttccagtacgccagcaccgacatggacaaggtgctgctcaagtacacggagtacaatgagccacacgagagccgcaccaacgccgacatcatcgagaccctgaggaagaagggcttcaatggctgcgacagccccgagcccgacggggaggactcgctggaacagagccccctgctggaggacaagtaccgacgcgccagcgaggagctcgacgggctcttccggcgctatgggtcaactgtcccggcccccaactttgccatgcctgtcacggtgcccgtgtccaatcagagctcactgcagttcagcaatcccagcggctccctggtcaccccttccctggtgacatcatccctcacggacccgcggctcctgtccccccagcagccagcactacagaggaacagtgtgtctcctggcctgccccagcggccagctagtgcgggggccatgctggggggtgacctgaacagtgctaacggagcctgccccagccctgttgggaatggctacgtcagtgctcgggcttcccctggcctcctccctgtggccaatggcaacagcctaaacaaggtcatccctgccaagtctccgcccccacctacccacagcacccagcttggagcccccagccgcaagcccgacctgcgagtcatcacttcccaggcaggaaaggggttaatgcatcacttgactgaggaccatttagatctgaacaatgcccagcgccttggggtctcccagtctactcattcgctcaccaccccagtggtttctgtggcaacgccgagtttactcagccagggcctccccttctcttccatgcccactgcctacaacacagattaccagttgaccagtgcagagctctcctccttaccagcctttagttcacctggggggctgtcgctaggcaatgtcactgcctggcaacagccacagcagccccagcagccgcagcagccacagcctccacagcagcagccaccgcagccacagcagccacagccacagcagcctcagcagccgcaacagccacctcagcaacagtcccacctggtccctgtatctctcagcaacctcatcccgggcagccccctgccccacgtgggtgctgccctcacagtcaccacccacccccacatcagcatcaagtcagaaccggtgtccccaagccgtgagcgcagccctgcgcctccccctccagctgtgttcccagctgcccgccctgagcctggcgatggtctcagcagcccagccgggggatcctatgagacgggagaccgggatgacggacggggggacttcgggcccacactgggcctgctgcgcccagccccagagcctgaggctgagggctcagctgtgaagaggatgcggcttgatacctggacattaaag

SEQ ID NO: 21 - protein, 217 aa, Rattus norvegicus
MSLVGGFPHHPVVHHEGYPFAAAAAAAAAAAASRCSHEENPYFHGWLIGHPEMSPPDYSMALSYSPEYASGAAGLDHSHYGGVPPGAGPPGLGGPRPVKRRGTANRKERRRTQSINSAFAELRECIPNVPADTKLSKIKTLRLATSYIAYLMDLLAKDDQNGEAEAFKAEIKKTDVKEEKRKKELNEILKSTVSSNDKKTKGRTGWPQHVWALELKQ

SEQ ID NO: 22 - DNA, 651 bp, Rattus norvegicus
  CDS: (1)..(654)
atgagtctggtggggggctttccccaccaccccgtggtgcaccatgagggctacccgttcgccgcagccgcagccgccgctgctgctgccgccgccagccgctgcagtcacgaggagaacccctatttccacggctggcttattggccacccggagatgtcgccccccgactacagcatggccctgtcctacagtcccgagtacgccagcggtgccgcgggcctggaccactcccattatgggggagtgccgcccggtgccgggcctcccggcctgggggggccgcgcccggtgaagcgtcggggcaccgccaaccgcaaggagcggcgcaggactcagagcatcaacagcgccttcgccgagctgcgcgagtgcatccccaacgtgcccgccgacaccaaactctccaaaatcaagactctgcgcctggccaccagctacatcgcctacctcatggatctgctggccaaggacgaccagaacggagaggcggaggccttcaaggcggagatcaagaagaccgacgtgaaagaggagaagaggaagaaagagctgaatgaaatcttgaaaagtacagtgagcagcaacgacaagaaaaccaaaggccggacaggctggccacagcacgtctgggccctggagctcaagcag

SEQ ID NO: 23 - protein, 215 aa, Homo sapiens
MNLVGSYAHHHHHHHPHPAHPMLHEPFLFGPASRCHQERPYFQSWLLSPADAAPDFPAGGPPPAAAAAATAYGPDARPGQSPGRLEALGGRLGRRKGSGPKKERRRTESINSAFAELRECIPNVPADTKLSKIKTLRLATSYIAYLMDVLAKDAQSGDPEAFKAELKKADGGRESKRKRELQQHEGFPPALGPVEKRIKGRTGWPQQVWALELNQ

SEQ ID NO: 24 - DNA, 645 bp, Homo sapiens
  CDS: (1)..(648)
atgaacctcgtgggcagctacgcacaccatcaccaccatcaccacccgcaccctgcgcaccccatgctccacgaacccttcctcttcggtccggcctcgcgctgtcatcaggaaaggccctacttccagagctggctgctgagcccggctgacgctgccccggacttccctgcgggcgggccgccgcccgcggccgctgcagccgccaccgcctatggtcctgacgccaggcctgggcagagccccgggcggctggaggcgcttggcggccgtcttggccggcggaaaggctcaggacccaagaaggagcggagacgcactgagagcattaacagcgcattcgcggagttgcgcgagtgcatccccaacgtgccggccgacaccaagctctccaagatcaagactctgcgcctagccaccagctacatcgcctacctgatggacgtgctggccaaggatgcacagtctggcgatcccgaggccttcaaggctgaactcaagaaggcggatggcggccgtgagagcaagcggaaaagggagctgcagcagcacgaaggttttcctcctgccctgggcccagtcgagaagaggattaaaggacgcaccggctggccgcagcaagtctgggcgctggagttaaaccag

SEQ ID NO: 25 - protein, 411 aa, Homo sapiens
MERMSDSADKPIDNDAEGVWSPDIEQSFQEALAIYPPCGRRKIILSDEGKMYGRNELIARYIKLRTGKTRTRKQVSSHIQVLARRKSRDFHSKLKDQTAKDKALQHMAAMSSAQIVSATAIHNKLGLPGIPRPTFPGAPGFWPGMIQTGQPGSSQDVKPFVQQAYPIQPAVTAPIPGFEPASAPAPSVPAWQGRSIGTTKLRLVEFSAFLEQQRDPDSYNKHLFVHIGHANHSYSDPLLESVDIRQIYDKFPEKKGGLKELFGKGPQNAFFLVKFWADLNCNIQDDAGAFYGVTSQYESSENMTVTCSTKVCSFGKQVVEKVETEYARFENGRFVYRINRSPMCEYMINFIHKLKHLPEKYMMNSVLENFTILLVVTNRDTQETLLCMACVFEVSNSEHGAQHHIYRLVKD

SEQ ID NO: 26 - DNA, 1233 bp, Homo sapiens
  CDS: (1)..(1236)
atggaaaggatgagtgactctgcagataagccaattgacaatgatgcagaaggggtctggagccccgacatcgagcaaagctttcaggaggccctggctatctatccaccatgtgggaggaggaaaatcatcttatcagacgaaggcaaaatgtatggtaggaatgaattgatagccagatacatcaaactcaggacaggcaagacgaggaccagaaaacaggtgtctagtcacattcaggttcttgccagaaggaaatctcgtgattttcattccaagctaaaggatcagactgcaaaggataaggccctgcagcacatggcggccatgtcctcagcccagatcgtctcggccactgccattcataacaagctggggctgcctgggattccacgcccgaccttcccaggggcgccggggttctggccgggaatgattcaaacagggcagccaggatcctcacaagacgtcaagccttttgtgcagcaggcctaccccatccagccagcggtcacagcccccattccagggtttgagcctgcatcggccccagctccctcagtccctgcctggcaaggtcgctccattggcacaaccaagcttcgcctggtggaattttcagcttttctcgagcagcagcgagacccagactcgtacaacaaacacctcttcgtgcacattgggcatgccaaccattcttacagtgacccattgcttgaatcagtggacattcgtcagatttatgacaaatttcctgaaaagaaaggtggcttaaaggaactgtttggaaagggccctcaaaatgccttcttcctcgtaaaattctgggctgatttaaactgcaatattcaagatgatgctggggctttttatggtgtaaccagtcagtacgagagttctgaaaatatgacagtcacctgttccaccaaagtttgctcctttgggaagcaagtagtagaaaaagtagagacggagtatgcaaggtttgagaatggccgatttgtataccgaataaaccgctccccaatgtgtgaatatatgatcaacttcatccacaagctcaaacacttaccagagaaatatatgatgaacagtgttttggaaaacttcacaattttattggtggtaacaaacagggatacacaagaaactctactctgcatggcctgtgtgtttgaagtttcaaatagtgaacacggagcacaacatcatatttacaggcttgtaaaggac

SEQ ID NO: 27 - protein, 427 aa, Homo sapiens
ITSNEWSSPTSPEGSTASGGSQALDKPIDNDAEGVWSPDIEQSFQEALAIYPPCGRRKIILSDEGKMYGRNELIARYIKLRTGKTRTRKQVSSHIQVLARRKAREIQAKLKDQAAKDKALQSMAAMSSAQIISATAFHSSMALARGPGRPAVSGFWQGALPGQAGTSHDVKPFSQQTYAVQPPLPLPGFESPAGPAPSPSAPPAPPWQGRSVASSKLWMLEFSAFLEQQQDPDTYNKHLFVHIGQSSPSYSDPYLEAVDIRQIYDKFPEKKGGLKDLFERGPSNAFFLVKFWADLNTNIEDEGSSFYGVSSQYESPENMIITCSTKVCSFGKQVVEKVETEYARYENGHYSYRIHRSPLCEYMINFIHKLKHLPEKYMMNSVLENFTILQVVTNRDTQETLLCIAYVFEVSASEHGAQHHIYRLVKE

SEQ ID NO: 28 - DNA, 1281 bp, Homo sapiens
  CDS: (1)..(1284)
attacctccaacgagtggagctctcccacctcccctgaggggagcaccgcctctgggggcagtcaggcactggacaagcccatcgacaatgacgcagagggcgtgtggagcccggatattgagcagagtttccaggaggccctcgccatctacccgccctgtggcaggcgcaaaatcatcctgtcggacgagggcaagatgtatggtcggaacgagctgattgcccgctacatcaagctccggacagggaagacccgcaccaggaagcaggtctccagccacatccaggtgctggctcgtcgcaaagctcgcgagatccaggccaagctaaaggaccaggcagctaaggacaaggccctgcagagcatggctgccatgtcgtctgcacagatcatctccgccacggccttccacagtagcatggccctcgcccggggccccggccgcccagcagtctcagggttttggcaaggagctttgccaggccaagccggaacgtcccatgatgtgaagcctttctctcagcaaacctatgctgtccagcctccgctgcctctgccagggtttgagtctcctgcagggcccgccccatcgccctctgcgcccccggcacccccatggcagggccgcagcgtggccagctccaagctctggatgttggagttctctgccttcctggagcagcagcaggacccggacacgtacaacaagcacctgttcgtgcacattggccagtccagcccaagctacagcgacccctacctcgaagccgtggacatccgccaaatctatgacaaattcccggagaaaaagggtggactcaaggatctcttcgaacggggaccctccaatgccttttttcttgtgaagttctgggcagacctcaacaccaacatcgaggatgaaggcagctccttctatggggtctccagccagtatgagagccccgagaacatgatcatcacctgctccacgaaggtctgctctttcggcaagcaggtggtggagaaagttgagacagagtatgctcgctatgagaatggacactactcttaccgcatccaccggtccccgctctgtgagtacatgatcaacttcatccacaagctcaagcacctccctgagaagtacatgatgaacagcgtgctggagaacttcaccatcctgcaggtggtcaccaacagagacacacaggagaccttgctgtgcattgcctatgtctttgaggtgtcagccagtgagcacggggctcagcaccacatctacaggctggtgaaagaa

SEQ ID NO: 29 - protein, 435 aa, Homo sapiens
IASNSWNASSSPGEAREDGPEGLDKGLDNDAEGVWSPDIEQSFQEALAIYPPCGRRKIILSDEGKMYGRNELIARYIKLRTGKTRTRKQVSSHIQVLARKKVREYQVGIKAMNLDQVSKDKALQSMASMSSAQIVSASVLQNKFSPPSPLPQAVFSTSSRFWSSPPLLGQQPGPSQDIKPFAQPAYPIQPPLPPTLSSYEPLAPLPSAAASVPVWQDRTIASSRLRLLEYSAFMEVQRDPDTYSKHLFVHIGQTNPAFSDPPLEAVDVRQIYDKFPEKKGGLKELYEKGPPNAFFLVKFWADLNSTIQEGPGAFYGVSSQYSSADSMTISVSTKVCSFGKQVVEKVETEYARLENGRFVYRIHRSPMCEYMINFIHKLKHLPEKYMMNSVLENFTILQVVTSRDSQETLLVIAFVFEVSTSEHGAQHHVYKLVKD

SEQ ID NO: 30 - DNA, 1305 bp, Homo sapiens
  CDS: (1)..(1305)
atagcgtccaacagctggaacgccagcagcagccccggggaggcccgggaggatgggcccgagggcctggacaaggggctggacaacgatgcggagggcgtgtggagcccggacatcgagcagagcttccaggaggccctggccatctacccgccctgcggccggcggaagatcatcctgtcagacgagggcaagatgtacggccgaaatgagttgattgcacgctatattaaactgaggacggggaagactcggacgagaaaacaggtgtccagccacatacaggttctagctcggaagaaggtgcgggagtaccaggttggcatcaaggccatgaacctggaccaggtctccaaggacaaagcccttcagagcatggcgtccatgtcctctgcccagatcgtctctgccagtgtcctgcagaacaagttcagcccaccttcccctctgccccaggccgtcttctccacttcctcgcggttctggagcagcccccctctcctgggacagcagcctggaccctctcaggacatcaagccctttgcacagccagcctaccccatccagccgcccctgccgccgacgctcagcagttatgagcccctggccccgctcccctcagctgctgcctctgtgcctgtgtggcaggaccgtaccattgcctcctcccggctgcggctcctggagtattcagccttcatggaggtgcagcgagaccctgacacgtacagcaaacacctgtttgtgcacatcggccagacgaaccccgccttctcagacccacccctggaggcagtagatgtgcgccagatctatgacaaattccccgagaaaaagggaggattgaaggagctctatgagaaggggccccctaatgccttcttccttgtcaagttctgggccgacctcaacagcaccatccaggagggcccgggagccttctatggggtcagctctcagtacagctctgctgatagcatgaccatcagcgtctccaccaaggtgtgctcctttggcaaacaggtggtagagaaggtggagactgagtatgccaggctggagaacgggcgctttgtgtaccgtatccaccgctcgcccatgtgcgagtacatgatcaacttcatccacaagctgaagcacctgcccgagaagtacatgatgaacagcgtgctggagaacttcaccatcctgcaggtggtcacgagccgggactcccaggagaccttgcttgtcattgcttttgtcttcgaagtctccaccagtgagcacggggcccagcaccatgtctacaagctcgtcaaagac

SEQ ID NO: 31 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 32 - DNA, 3396 bp, Homo sapiens
  CDS: (1)..(3399)
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggac

SEQ ID NO: 33 - DNA, 21 bp, Artificial Sequence
  Description of Artificial Sequence artificially synthesized primer sequence
ttggcttccaggccataattg

SEQ ID NO: 34 - DNA, 20 bp, Artificial Sequence
  Description of Artificial Sequence artificially synthesized primer sequence
aagagggcagatctatcgga

SEQ ID NO: 35 - DNA, 20 bp, Artificial Sequence
  Description of Artificial Sequence artificially synthesized primer sequence
atggatctcctgaaggtgct

SEQ ID NO: 36 - DNA, 20 bp, Artificial Sequence
  Description of Artificial Sequence artificially synthesized primer sequence
aagagggcagatctatcgga

SEQ ID NO: 37 - DNA, 23 bp, Artificial Sequence
  Description of Artificial Sequence artificially synthesized primer sequence
ggaagagtgagcggccatcaagg

SEQ ID NO: 38 - DNA, 22 bp, Artificial Sequence
  Description of Artificial Sequence artificially synthesized primer sequence
ctgctggagaggttattcctcg

SEQ ID NO: 39 - DNA, 24 bp, Artificial Sequence
  Description of Artificial Sequence artificially synthesized primer sequence
gccaacaccaacctgtccaagttc

SEQ ID NO: 40 - DNA, 24 bp, Artificial Sequence
  Description of Artificial Sequence artificially synthesized primer sequence
tgcaaaggctccaggtctgagggc

SEQ ID NO: 41 - DNA, 19 bp, Artificial Sequence
  Description of Artificial Sequence artificially synthesized primer sequence
ctctctctcctcaggacaa

SEQ ID NO: 42 - DNA, 22 bp, Artificial Sequence
  Description of Artificial Sequence artificially synthesized primer sequence
tggagcaaaacagaatggctgg

SEQ ID NO: 43 - DNA, 24 bp, Artificial Sequence
  Description of Artificial Sequence artificially synthesized primer sequence
ctgagatgtctctctctctcttag

SEQ ID NO: 44 - DNA, 20 bp, Artificial Sequence
  Description of Artificial Sequence artificially synthesized primer sequence
acaatgactgatgagagatg

SEQ ID NO: 45 - DNA, 18 bp, Artificial Sequence
  Description of Artificial Sequence artificially synthesized primer sequence
cagacctgaaggagacct

SEQ ID NO: 46 - DNA, 18 bp, Artificial Sequence
  Description of Artificial Sequence artificially synthesized primer sequence
gtcagcgtaaacagttgc

SEQ ID NO: 47 - DNA, 20 bp, Artificial Sequence
  Description of Artificial Sequence artificially synthesized primer sequence
gccaagaagcggatagaagg

SEQ ID NO: 48 - DNA, 20 bp, Artificial Sequence
  Description of Artificial Sequence artificially synthesized primer sequence
ctgtggttcagggctcagtc

SEQ ID NO: 49 - DNA, 20 bp, Artificial Sequence
  Description of Artificial Sequence artificially synthesized primer sequence
cagtggagctggacaaagcc

SEQ ID NO: 50 - DNA, 20 bp, Artificial Sequence
  Description of Artificial Sequence artificially synthesized primer sequence
tagcgacggttctggaacca

SEQ ID NO: 51 - DNA, 20 bp, Artificial Sequence
  Description of Artificial Sequence artificially synthesized primer sequence
ctgtcatctcactatgggca

SEQ ID NO: 52 - DNA, 20 bp, Artificial Sequence
  Description of Artificial Sequence artificially synthesized primer sequence
ccaagtccgagcaggaattt

SEQ ID NO: 53 - DNA, 20 bp, Artificial Sequence
  Description of Artificial Sequence artificially synthesized primer sequence
aagacgtcaagccctttgtg

SEQ ID NO: 54 - DNA, 20 bp, Artificial Sequence
  Description of Artificial Sequence artificially synthesized primer sequence
aaaggagcacactttggtgg

SEQ ID NO: 55 - DNA, 20 bp, Artificial Sequence
  Description of Artificial Sequence artificially synthesized primer sequence
agcaagaatacgatgccatc

SEQ ID NO: 56 - DNA, 20 bp, Artificial Sequence
  Description of Artificial Sequence artificially synthesized primer sequence
gaaggggtggtggtacggtc

SEQ ID NO: 57 - DNA, 20 bp, Artificial Sequence
  Description of Artificial Sequence artificially synthesized primer sequence
tgggaatggctatgtcagtg

SEQ ID NO: 58 - DNA, 20 bp, Artificial Sequence
  Description of Artificial Sequence artificially synthesized primer sequence
ctggtaatctgtgttgtagg

SEQ ID NO: 59 - DNA, 20 bp, Artificial Sequence
  Description of Artificial Sequence artificially synthesized primer sequence
caagggcctctccaaacttg

SEQ ID NO: 60 - DNA, 20 bp, Artificial Sequence
  Description of Artificial Sequence artificially synthesized primer sequence
gccccagagacagcattcca

SEQ ID NO: 61 - protein, 268 aa, Homo sapiens
MAQPLCPPLSESWMLSAAWGPTRRPPPSDKDCGRSLVSSPDSWGSTPADSPVASPARPGTLRDPRAPSVGRRGARSSRLGSGQRQSASEREKLRMRTLARALHELRRFLPPSVAPAGQSLTKIETLRLAIRYIGHLSAVLGLSEESLQRRCRQRGDAGSPRGCPLCPDDCPAQMQTRTQAEGQGQGRGLGLVSAVRAGASWGSPPACPGARAAPEPRDPPALFAEAACPEGQAMEPSPPSPLLPGDVLALLETWMPLSPLEWLPEEPK

SEQ ID NO: 62 - DNA, 804 bp, Homo sapiens
  CDS: (1)..(807)
atggcccagcccctgtgcccgccgctctccgagtcctggatgctctctgcggcctggggcccaactcggcggccgccgccctccgacaaggactgcggccgctccctcgtctcgtccccagactcatggggcagcaccccagccgacagccccgtggcgagccccgcgcggccaggcaccctccgggacccccgcgccccctccgtaggtaggcgcggcgcgcgcagcagccgcctgggcagcgggcagaggcagagcgccagtgagcgggagaaactgcgcatgcgcacgctggcccgcgccctgcacgagctgcgccgctttctaccgccgtccgtggcgcccgcgggccagagcctgaccaagatcgagacgctgcgcctggctatccgctatatcggccacctgtcggccgtgctaggcctcagcgaggagagtctccagcgccggtgccggcagcgcggtgacgcggggtcccctcggggctgcccgctgtgccccgacgactgccccgcgcagatgcagacacggacgcaggctgaggggcaggggcaggggcgcgggctgggcctggtatccgccgtccgcgccggggcgtcctggggatccccgcctgcctgccccggagcccgagctgcacccgagccgcgcgacccgcctgcgctgttcgccgaggcggcgtgccctgaagggcaggcgatggagccaagcccaccgtccccgctccttccgggcgacgtgctggctctgttggagacctggatgcccctctcgcctctggagtggctgcctgaggagcccaag

SEQ ID NO: 63 - protein, 215 aa, Homo sapiens
MGSPRSALSCLLLHLLVLCLQAQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 64 - DNA, 645 bp, Homo sapiens
  CDS: (1)..(648)
atgggcagcccccgctccgcgctgagctgcctgctgttgcacttgctggtcctctgcctccaagcccaggtaactgttcagtcctcacctaattttacacagcatgtgagggagcagagcctggtgacggatcagctcagccgccgcctcatccggacctaccaactctacagccgcaccagcgggaagcacgtgcaggtcctggccaacaagcgcatcaacgccatggcagaggacggcgaccccttcgcaaagctcatcgtggagacggacacctttggaagcagagttcgagtccgaggagccgagacgggcctctacatctgcatgaacaagaaggggaagctgatcgccaagagcaacggcaaaggcaaggactgcgtcttcacggagattgtgctggagaacaactacacagcgctgcagaatgccaagtacgagggctggtacatggccttcacccgcaagggccggccccgcaagggctccaagacgcggcagcaccagcgtgaggtccacttcatgaagcggctgccccggggccaccacaccaccgagcagagcctgcgcttcgagttcctcaactacccgcccttcacgcgcagcctgcgcggcagccagaggacttgggccccggaaccccga

SEQ ID NO: 65 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 66 - DNA, 636 bp, Homo sapiens
  CDS: (1)..(639)
atggattatttgctcatgattttctctctgctgtttgtggcttgccaaggagctccagaaacagcagtcttaggcgctgagctcagcgcggtgggtgagaacggcggggagaaacccactcccagtccaccctggcggctccgccggtccaagcgctgctcctgctcgtccctgatggataaagagtgtgtctacttctgccacctggacatcatttgggtcaacactcccgagcacgttgttccgtatggacttggaagccctaggtccaagagagccttggagaatttacttcccacaaaggcaacagaccgtgagaatagatgccaatgtgctagccaaaaagacaagaagtgctggaatttttgccaagcaggaaaagaactcagggctgaagacattatggagaaagactggaataatcataagaaaggaaaagactgttccaagcttgggaaaaagtgtatttatcagcagttagtgagaggaagaaaaatcagaagaagttcagaggaacacctaagacaaaccaggtcggagaccatgagaaacagcgtcaaatcatcttttcatgatcccaagctgaaaggcaagccctccagagagcgttatgtgacccacaaccgagcacattgg

SEQ ID NO: 67 - protein, 143 aa, Homo sapiens
MQHRGFLLLTLLALLALTSAVAKKKDKVKKGGPGSECAEWAWGPCTPSSKDCGVGFREGTCGAQTQRIRCRVPCNWKKEFGADCKYKFENWGACDGGTGTKVRQGTLKKARYNAQCQETIRVTKPCTPKTKAKAKAKKGKGKD

SEQ ID NO: 68 - DNA, 429 bp, Homo sapiens
  CDS: (1)..(432)
atgcagcaccgaggcttcctcctcctcaccctcctcgccctgctggcgctcacctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggac

SEQ ID NO: 69 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 70 - DNA, 1224 bp, Homo sapiens
  CDS: (1)..(1227)
atgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggcgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgc

SEQ ID NO: 71 - DNA, 24 bp, Artificial Sequence
gcccgcgctccaactgctctgatg

SEQ ID NO: 72 - DNA, 24 bp, Artificial Sequence
tgcctacggtggtgcgccctctgc

SEQ ID NO: 73 - DNA, 22 bp, Artificial Sequence
gaagcgcaacagggccatcacg

SEQ ID NO: 74 - DNA, 22 bp, Artificial Sequence
ccacgtcacgcaggtcccgttc

SEQ ID NO: 75 - DNA, 22 bp, Artificial Sequence
gatcctgttctctgcctctgga

SEQ ID NO: 76 - DNA, 22 bp, Artificial Sequence
tcatccactttgtccacccgag

SEQ ID NO: 77 - DNA, 21 bp, Artificial Sequence
ttcctcgtcttggccttttgg

SEQ ID NO: 78 - DNA, 21 bp, Artificial Sequence
gctggatcttcgtaggctccg

SEQ ID NO: 79 - DNA, 19 bp, Artificial Sequence
ggcaagctgaccctgaagt

SEQ ID NO: 80 - DNA, 19 bp, Artificial Sequence
gggtgctcaggtagtggtt

(19) United States
(12) Patent Application Publication
(10) Pub. No.: US 2002/0142457 A1
(43) Pub. Date: Oct. 3, 2002

(54) Cell having the potentiality of differentiation into cardiomyocytes

(76) Inventors: Akihiro Umezawa, Chiba (JP); Jun-Ichi Hata, Tokyo (JP); Keiichi Fukuda, Tokyo (JP); Satoshi Ogawa, Tokyo (JP); Kazuhiro Sakurada, Tokyo (JP); Satoshi Gojo, Saitama (JP); Yoji Yamada, Tokyo (JP)

Correspondence Address: FITZPATRICK CELLA HARPER & SCINTO, 30 ROCKEFELLER PLAZA, NEW YORK, NY, 10112, US

(21) Appl. No.: 09/749,728

(22) Filed: Dec. 28, 2000

Related U.S. Application Data
(60) Provisional application No. 60/244,594, filed on Nov. 1, 2000.

(30) Foreign Application Priority Data
Dec. 28, 1999 (JP) HEI. 11-372826
Feb. 28, 2000 (WO) PCT/JP00/01148

(51) Int. Cl. C12N 5/08

(52) U.S. Cl. 435/366

(57) ABSTRACT

The present invention relates to methods for isolation, purification, expansion, and differentiation of cells having the potential to differentiate into cardiomyocytes. Furthermore, the present invention relates to methods for proliferating cells having the potential to differentiate into cardiomyocytes and for regulating their differentiation into cardiomyocytes using various cytokines and transcription factors. Moreover, the present invention relates to a method for obtaining surface antigens specific for cells having the potential to differentiate into cardiomyocytes, a method for obtaining genes encoding the surface antigens, a method for obtaining antibodies specific for the surface antigens, and a method for obtaining a protein and a gene controlling the proliferation of cells having the potential to differentiation into cardiomyocytes and their differentiation into cardiomyocytes. Also, the present invention relates to therapeutic agents for various heart diseases containing cells having the potential to differentiate into cardiomyocytes. Still furthermore, the present invention relates to a method for differentiating various cells and tissues such as neural cells, hepatocytes, adipocytes, skeletal muscle cells, vascular endothelial cells and osteoblasts, using cells having the potential to differentiate into cardiomyocytes.

### TECHNICAL FIELD

The present invention relates to methods for isolation, purification, expansion, and differentiation of cells having the potential to differentiate into cardiomyocytes. Furthermore, the present invention relates to methods for proliferating cells having the potential to differentiate into cardiomyocytes and for regulating their differentiation into cardiomyocytes using various cytokines and transcription factors. Moreover, the present invention relates to a method for obtaining surface antigens specific for cells having the potential to differentiate into cardiomyocytes, a method for obtaining genes encoding the surface antigens, a method for obtaining antibodies specific for the surface antigens, and a method for obtaining a protein and a gene controlling the proliferation of cells having the potential to differentiate into cardiomyocytes and their differentiation into cardiomyocytes. Also, the present invention relates to therapeutic agents for various heart diseases containing cells having the potential to differentiate into cardiomyocytes.

### BACKGROUND ART

Cardiomyocytes actively divide into daughter cells with spontaneous beating before birth. However, they lose the proliferative activity after birth and never acquire the division potentiality again unlike hepatocytes. Furthermore, unlike skeletal muscles, they does not have undifferentiated precursor cells such as satellite cells. Therefore, when cardiomyocytes are necrotized by myocardial infarction, myocarditis, senility etc., hypertrophy of the remaining cardiomyocytes occurs in vivo instead of cell division. Cardiac hypertrophy is a physiological adaptation at the initial stage, but when coupled with the fibrosis of stroma by the growth of cardiac fibroblasts, it comes to lower the diastolic function and the systolic function of heart itself, leading to heart failure. Therapy so far developed for heart failure caused by myocardial infarction, etc. is mainly symptomatic therapy, for example, intensification of the cardiac systolic function, alleviation of the pressure overload and the volume load on heart using a vasodilator drug, and decrease of blood flow using of a diuretic. On the other hand, heart transplantation is alternative therapy for severe heart failure, but is not generally adopted as a common treatment because of problems such as shortage of heart donors, difficulty in judging cerebral death, immune rejection and a great rise in medical cost. At present, heart diseases are the third cause of mortality in Japan (Annual Report on Health and Welfare, 1998), and thus success in regeneration of lost cardiomyocytes will lead to a great advance in medical welfare.

As a cell line retaining the characteristics of cardiomyocytes, AT-1 cell line has been obtained from the atrial tumor of the transgenic mouse expressing SV40 promoter large T antigen under the control of atrial natriuretic hormone promoter (Science, 239: 1029-1038 (1988)). However, this cell line forms tumors when transplanted in vivo and thus is inappropriate for cell transplantation. Under these circumstances, the following methods have been proposed for reconstructing myocardium.

The first method is conversion of cells other than cardiomyocytes into cardiomyocytes, which has been proposed on the analogy of the conversion of fibroblasts into skeletal muscle cells by the introduction of MyoD. Although a successful result has been reported with P19 cell which is a murine embryonal carcinoma cell (Cell Struc. & Func., 21: 101-110 (1996)), there has been no report on success with non-carcinomatous cells.

The second method is restoration of proliferative activity to cardiomyocytes, which is based on the fact that beating cardiomyocytes can proliferate in the fetus. No successful example of this method has been reported yet.

The third method is induction of cardiomyocytes from undifferentiated stem cells. It has already been demonstrated that cardiomyocytes can be differentiated from embryonic stem cells (ES cells), but there still remain the problems of carcinoma formation and immune rejection by embryonic stem cells transplanted into an adult tissue. (Nature Biotechnology, 17: 139-142 (1999)).

In order to practically utilize embryonic stem cells in medical treatments, it is essential to develop a technique for highly purifying at least cardiomyocyte precursor cells or cardiomyocytes. As for the problem of immune rejection, the possibility of solving the problem by the cloning technique has been suggested, but it is difficult to apply this technique to general medical treatments because of its complicated operation.

It has also been proposed to transplant undifferentiated cardiomyocyte precursor cells obtained from an aborted fetus, and it is known that such cells effectively function as cardiomyocytes in an experiment using animals (Science, 264: 98-101 (1994)). However, it is difficult to obtain a large amount of cardiomyocyte precursor cells in this method, and the method is hardly applicable to general medical treatments also from an ethical viewpoint.

It is known that there exist mesenchymal stem cells besides hematopoietic stem cells and vascular stem cells in adult bone marrow and that mesenchymal stem cells can be induced to differentiate into osteocytes, chondrocytes, tendon cells, ligament cells, skeletal muscle cells, adipocytes, stromal cells and hepatic oval cells (Science, 284: 143-147 (1999); Science, 284: 1168-1170 (1999)). On the other hand, it has been recently reported that the cells obtained from the bone marrow of an adult mouse can be induced to differentiate into cardiomyocytes (J. Clinical Investigation, 103: 10-18 (1999)). This report suggests that the cell therapy which comprises transplanting cells which are obtained from bone marrow fluid taken from a patient followed by in vitro expansion and drug treatment to the damaged part of the patient's heart can be a practical medical treatment (J. Clinical Investigation, 103: 591-592 (1999)). However, this report merely indicates that a part of the immortalized cells established from the bone marrow of an adult mouse can differentiate into cardiomyocytes. Furthermore, the report fails to isolate, selectively proliferate, and efficiently differentiate the adult bone marrow cells having the potential to differentiate into cardiomyocytes (J. Clinical Investigation, 103: 591-592 (1999)).

Antibodies which recognize various surface antigens are used to isolate the target cells from the tissue of vital body. For example, it is known that immature hematopoietic stem cells have the characteristics of CD34+/CD38-/HLA-DR-/CD90 (Thy-1)+, and CD38 is expressed while CD90(Thy-1) disappears in the process of differentiation (Protein, Nucleic Acid, Enzyme, 45: 13, 2056-2062 (2000)). In vascular endothelial cells, markers such as CD34, CD31, Flk-1, Tie-2, E-selectin, etc. are expressed (Molecular Cardiovascular Disease, 1(3): 294-302 (2000)). In bone marrow mesenchymal stem cells, markers such as CD90, CD105, CD140, etc. are expressed (Science, 284: 143-147 (1999); Science, 284: 1168-1170 (1999)). However, no surface marker of stem cells capable of inducing both myocardium and vascular endothelial cells is known.

### DISCLOSURE OF THE INVENTION

Under the circumstances, a need exists for the development of therapy for heart diseases which therapy is safer and more established than those currently available. It is useful to select cells having the potential to differentiate into cardiomyocytes from a vital tissue such as bone marrow cells or the like or umbilical blood and to control the growth or differentiation of the cells for the development of myocardium-regenerating therapy using vital cells such as bone marrow-derived cells or the like or umbilical blood. For this purpose, it is necessary to separate the cells having the potential to differentiate into cardiomyocytes and to identify cytokines or transcription factors participating in the growth or differentiation of such cells.

The present inventors have made intensive studies aiming at solving the above problems and have obtained the following results. Specifically, various cell lines were obtained by separating mouse bone marrow-derived cells to single cell level. Then, various cell lines have characterized by their potential to differentiate into cardiomyocytes by treating each cell line with 5-azacytidine. Next, by labeling the thus obtained cell line using a retrovirus vector which expresses a GFP (green fluorescent protein) and tracing the cells using a fluorescence microscope, it has been found that the bone marrow-derived cells are pluripotent stem cells which can differentiate into at least two different cells, i.e., cardiomyocytes and adipocytes. Furthermore, it has been found that the stem cells can be differentiated into cardiomyocytes, adipocytes and skeletal muscle cells stochastically by addition of not only 5-azacytidine but also other genomic DNA-demethylating agents, such as DMSO (dimethyl sulfoxide), indicating that demethylation of genomic DNA is effective in inducing the differentiation of bone marrow-derived cells into cardiomyocytes. Moreover, it was found that the expression of myocardium-specific genes, ANP (atrral natriuretic peptide) and cTnI (cardiac Troponin I), can be expressed in the bone marrow-derived cells by adding at least one cytokine of four cytokines, FGF-8, ET1, midkine and BMP4, combined with 5-azacytidine. Also, it was found that differentiation of the bone marrow-derived cells into cardiomyocytes can be promoted about 50-fold by the forced expression of two transcriptional factors, Nkx2.5 and GATA4, in these bone marrow-derived cells using virus vectors followed by 5-azacytidine treatment. Furthermore, it was found that the expression of ANP and cTnI, which are myocardium-specific genes, in the bone marrow-derived cells can be specifically promoted by culturing these bone marrow-derived cells in a culture dish coated with a cardiomyocyte-derived extracellular substrate. Moreover, it was found that the formation of myocardium from the bone marrow-derived cells can be about 10 times or more promoted by co-culturing the bone marrow-derived cells together with primarily cultured cells derived from myocardium. Moreover, it was found that differentiation of the bone marrow-derived cells into cardiomyocytes can be promoted about 500-fold when the forced expression of two transcription factors Nkx2.5 and GATA4 in the bone marrow-derived cells using virus vectors and co-culturing these cells with cardiomyocytes were combined.

Subsequently, the differentiation potency of the bone marrow-derived cells was examined by a transplantation experiment. First, the bone marrow-derived cells were transplanted into an adult mouse heart and it was thus found that these bone marrow-derived cells were differentiated into myocardia and vessels. Furthermore, the bone marrow-derived cells were transplanted into an adult mouse muscle and it was thus found that these bone marrow-derived cells could form skeletal muscles. When the bone marrow-derived cells were transplanted into a mouse blastocyst, tissues derived from these transplanted cells were formed in the central nervous system, liver and heart of the mouse. The central nervous system, liver and heart are tissues of the ectoderm, endoderm and mesoderm, respectively.

These results indicate that the bone marrow-derived cells found in the present invention have properties different from those possessed by hematopoietic stem cells which are differentiated into only hematopoietic stem tissue present in bone marrow and from those possessed by mesenchymal stem cell which are differentiated into only dorsal mesoderm tissue such as skeletal muscle, adipocytes, bone and the like known in the art, that is, a totipotency of differentiating into all of the three germ layers including the ectoderm, mesoderm and endoderm.

Furthermore, the inventors analyzed the expression of surface antigens of bone marrow-derived cells using antibodies which recognize hematopoietic cell surface antigens, CD34, CD117, CD14, CD45, CD90, Sca-1, Ly6c and Ly6g, antibodies which recognize vascular endothelial cell surface antigens, Flk-1, CD31, CD105 and CD144, antibodies which recognize a mesenchymal cell surface antigen, CD140, antibodies which recognize integrin surface antigens, CD49b, CD49d, CD29 and CD41, and antibodies which recognize matrix receptors, CD54, CD102, CD106 and CD44, and the like in these bone marrow cells of the present invention and thus found that they are totipotential stem cells exhibiting a quite novel expression form having been unknown, thereby completing the present invention.

Specifically, the present invention provides the following (1)-(91):

(1) A cell which has been isolated from a living tissue or umbilical blood, and which has the potential to differentiate into at least a cardiomyocyte.

(2) The cell according to (1), wherein the living tissue is bone marrow.

(3) The cell according to (1) or (2), wherein the cell is a multipotential stem cell.

(4) The cell according to any one of (1) to (3), wherein the cell is a multipotential stem cell which differentiates into at least a cardiomyocyte and a vascular endothelial cell.

(5) The cell according to any one of (1) to (4), wherein the cell is a multipotential stem cell which differentiates into at least a cardiomyocyte, an adipocyte, a skeletal muscle cell, an osteoblast, and a vascular endothelial cell.

(6) The cell according to any one of (1) to (5), wherein the cell is a multipotential stem cell which differentiates into at least a cardiomyocyte, an adipocyte, a skeletal muscle cell, an osteoblast, a vascular endothelial cell, a nervous cell, and a hepatic cell.

(7) The cell according to any one of (1) to (3), wherein the cell is a multipotential stem cell which differentiates into any cell in adult tissues.

(8) The cell according to any one of (1) to (7), wherein the cell is CD117-positive and CD140-positive.

(9) The cell according to (8), wherein the cell is further CD34-positive.

(10) The cell according to (9), wherein the cell is further CD144-positive.

(11) The cell according to (9), wherein the cell is further CD140-negative.

(12) The cell according to (8), wherein the cell is CD34-negative.

(13) The cell according to (12), wherein the cell is further CD144-positive.

(14) The cell according to (12), wherein the cell is further CD144-negative.

(15) The cell according to (10), wherein the cell is further CD14-negative, CD45-negative, CD90-negative, Flk-1-negative, CD31-negative, CD105-negative, CD49b-negative, CD49d-negative, CD29-positive, CD54-negative, CD102-negative, CD106-negative, and CD44-positive.

(16) The cell according to (11), wherein the cell is further CD14-negative, CD45-negative, CD90-negative, Flk-1-negative, CD31-negative, CD105-negative, CD49b-negative, CD49d-negative, CD29-positive, CD54-negative, CD102-negative, CD106-negative, and CD44-positive.

(17) The cell according to (12), wherein the cell is further CD14-negative, CD45-negative, CD90-negative, Flk-1-negative, CD31-negative, CD105-negative, CD49b-negative, CD49d-negative, CD29-positive, CD54-negative, CD102-negative, CD106-negative, and CD44-positive.

(18) The cell according to (13), wherein the cell is further CD14-negative, CD45-negative, CD90-negative, Flk-1-negative, CD31-negative, CD105-negative, CD49b-negative, CD49d-negative, CD29-positive, CD54-negative, CD102-negative, CD106-negative, and CD44-positive.

(19) The cell according to (1), which does not take up Hoechst 33342.

(20) A cardiomyocyte precursor which differentiates into only cardiomyocyte induced from the cell according to any one of (1) to (19).

(21) The cell according to any one of (1) to (20), which has the potential to differentiate into a ventricular cardiac muscle cell.

(22) The cell according to any one of (1) to (20), which has the potential to differentiate into a sinus node cell.

(23) The cell according to any one of (1) to (20), wherein the vital tissue or umbilical blood is derived from a mammal.

(24) The cell according to (23), wherein the mammal is selected from the group consisting of a mouse, a rat, a guinea pig, a hamster, a rabbit, a cat, a dog, a sheep, a swine, cattle, a goat and a human.

(25) The cell according to any one of (1) to (8), which is mouse bone marrow-derived multipotential stem cell BMSC (FERM BP-7043).

(26) The cell according to any one of (1) to (25), which has the potential to differentiate into a cardiomyocyte by demethylation of a chromosomal DNA of the cell.

(27) The cell according to (26), wherein the demethylation is carried out by at least one selected from the group consisting of demethylase, 5-azacytidine, and dimethyl sulfoxide, DMSO.

(28) The cell according to (27), wherein the demethylase comprises the amino acid sequence represented by SEQ ID NO:1.

(29) The cell according to any one of (1) to (28), wherein the differentiation is accelerated by a factor which is expressed in a cardiogenesis region of a fetus or a factor which acts on differentiation into a cardiomyocyte in a cardiogenesis stage of a fetus.

(30) The cell according to (29), wherein the factor which is expressed in a cardiogenesis region of a fetus or the factor which acts on differentiation into a cardiomyocyte in a cardiogenesis stage of a fetus is at least one selected from the group consisting of a cytokine, an adhesion molecule, a vitamin, a transcription factor, and an extracellular matrix.

(31) The cell according to (30), wherein the cytokine is at least one selected from the group consisting of a platelet-derived growth factor, PDGF; a fibroblast growth factor-8, FGF-8; an endothelin 1, ET1; a midkine; and a bone morphogenetic factor, BMP-4.

(32) The cell according to (31), wherein the PDGF, FGF-8, ET1, midkine, and BMP-4 comprise the amino acid sequence represented by SEQ ID NO:3 or 5, the amino acid sequence represented by SEQ ID NO:64, the amino acid sequence represented by SEQ ID NO:66, the amino acid sequence represented by SEQ ID NO:68, and the amino acid sequence represented by SEQ ID NO:70, respectively.

(33) The cell according to (30), wherein the adhesion molecule is at least one selected from the group consisting of a gelatin, a laminin, a collagen, and a fibronectin.

(34) The cell according to (30), wherein the vitamin is retinoic acid.

(35) The cell according to (30), wherein the transcription factor is at least one selected from the group consisting of Nkx2)5/Csx, GATA4, MEF-2A, MEF-2B, MEF-2C, MEF-2D, dHAND, eHAND, TEF-1, TEF-3, TEF-5, and MesP1.

(36) The cell according to (35), wherein the Nkx2)5/Csx, GATA4, MEF-2A, MEF-2B, MEF-2C, MEF-2D, dHAND, eHAND, TEF-1, TEF-3, TEF-5, and MesP1 comprise the amino acid sequence represented by SEQ ID NO:9, the amino acid sequence represented by SEQ ID NO:11, the amino acid sequence represented by SEQ ID NO:13, the amino acid sequence represented by SEQ ID NO:15, the amino acid sequence represented by SEQ ID NO:17, the amino acid sequence represented by SEQ ID NO:19, the amino acid sequence represented by SEQ ID NO:21, the amino acid sequence represented by SEQ ID NO:23, the amino acid sequence represented by SEQ ID NO:25, the amino acid sequence represented by SEQ ID NO:27, the amino acid sequence represented by SEQ ID NO:29, and the amino acid sequence represented by SEQ ID NO:62, respectively.

(37) The cell according to (30), wherein the extracellular matrix is an extracellular matrix derived from a cardiomyocyte.

(38) The cell according to any one of (1) to (28), wherein the differentiation is inhibited by a fibroblast growth factor-2, FGF-2.

(39) The cell according to (38), wherein the FGF-2 comprises the amino acid sequence represented by SEQ ID NO:7 or 8.

(40) The cell according to any one of (1) to (28), which is capable of differentiating into a cardiomyocyte or a blood vessel by transplantation into a heart.

(41) The cell according to any one of (1) to (28), which is capable of differentiating into a cardiac muscle by transplantation into a blastocyst or by co-culturing with a cardiomyocyte.

(42) The cell according to any one of (1) to (28), which is capable of differentiating into an adipocyte by an activator of a nuclear receptor, PPAR-Y.

(43) The cell according to (42), wherein the activator is a compound having a thiazolidione skeleton.

(44) The cell according to (43), wherein the compound is at least one selected from the group consisting of troglitazone, pioglitazone, and rosiglitazone.

(45) The cell according to any one of (1) to (28), which is capable of differentiating into a nervous cell by transplantation into a blastocyst or by transplantation into an encephalon or a spinal cord.

(46) The cell according to any one of (1) to (28), which is capable of differentiating into a hepatic cell by transplantation into a blastocyst or by transplantation into a liver.

(47) A method for differentiting the cell according to any one of (1) to (28) into a cardiac muscle, comprising using a chromosomal DNA-dimethylating agent.

(48) A method for redifferentiating the cell according to (9) into the cell according to (12), comprising using a chromosomal DNA-dimethylating agent.

(49) A method for redifferentiating a cell which is CD117-negative and CD140-positive into the cell according to (8), comprising using a chromosomal DNA-dimethylating agent.

(50) The method according to (48) or (49), wherein the chromosomal DNA-dimethylating agent is selected from the group consisting of a demethylase, 5-azacytidine, and DMSO.

(51) The method according to (50), wherein the demethylase comprises the amino acid sequence represented by SEQ ID NO:1.

(52) A method for differentiating the cell according to any one of (1) to (28) into a cardiac muscle, comprising using a factor which is expressed in a cardiogenesis region of a fetus or a factor which acts on differentiation into a cardiomyocyte in a cardiogenesis stage of a fetus.

(53) The method according to (52), wherein the factor which is expressed in a cardiogenesis region of a fetus or the factor which acts on differentiation into a cardiomyocyte in a cardiogenesis stage of a fetus is at least one selected from the group consisting of a cytokine, an adhesion molecule, a vitamin, a transcription factor, and an extracellular matrix.

(54) The method according to (53), wherein the cytokine is at least one selected from the group consisting of a platelet-derived growth factor, PDGF; a fibroblast growth factor-8, FGF-8; an endothelin 1, ET1; a midkine; and a bone morphogenetic factor, BMP-4.

(55) The method according to (54), wherein the PDGF, FGF-8, ET1, midkine, and BMP-4 comprise the amino acid sequence represented by SEQ ID NO:3 or 5, the amino acid sequence represented by SEQ ID NO:64, the amino acid sequence represented by SEQ ID NO:66, the amino acid sequence represented by SEQ ID NO:68, and the amino acid sequence represented by SEQ ID NO:70, respectively.

(56) The method according to (53), wherein the adhesion molecule is at least one selected from the group consisting of a gelatin, a laminin, a collagen, and a fibronectin.

(57) The method according to (53), wherein the vitamin is retinoic acid.

(58) The method according to (53), wherein the transcription factor is at least one selected from the group consisting of Nkx2)5/Csx, GATA4, MEF-2A, MEF-2B, MEF-2C, MEF-2D, dHAND, eHAND, TEF-1, TEF-3, TEF-5, and MesP1.

(59) The method according to (58), wherein the Nkx2)5/Csx, GATA4, MEF-2A, MEF-2B, MEF-2C, MEF-2D, dHAND, eHAND, TEF-1, TEF-3, TEF-5, and MesP1 comprise the amino acid sequence represented by SEQ ID NO:9, the amino acid sequence represented by SEQ ID NO:11, the amino acid sequence represented by SEQ ID NO:13, the amino acid sequence represented by SEQ ID NO:15, the amino acid sequence represented by SEQ ID NO:17, the amino acid sequence represented by SEQ ID NO:19, the amino acid sequence represented by SEQ ID NO:21, the amino acid sequence represented by SEQ ID NO:23, the amino acid sequence represented by SEQ ID NO:25, the amino acid sequence represented by SEQ ID NO:27, the amino acid sequence represented by SEQ ID NO:29, the amino acid sequence represented by SEQ ID NO:62, respectively.

(60) The method according to (53), wherein the extracellular matrix is an extracellular matrix derived from a cardiomyocyte. (61) A method for differentiating the cell according to any one of (1) to (28) into an adipocyte, comprising using an activator of a nuclear receptor , PPAR-γ.

(62) The method according to (61), wherein the activator is a compound having a thiazolidione skeleton.

(63) The method according to (62), wherein the compound is at least one selected from the group consisting of troglitazone, pioglitazone, and rosiglitazone.

(64) A myocardium-forming agent, comprising, as an active ingredient, a chromosomal DNA-demethylating agent.

(65) The myocardium-forming agent according to (64), wherein the chromosomal DNA-demethylating agent is at least one selected from the group consisting of a demethylase, 5-azacytidine, and DMSO.

(66) The myocardium-forming agent according to (65), wherein the demethylase comprises the amino acid sequence represented by SEQ ID NO:1.

(67) A myocardium-forming agent, comprising, as an active ingredient, a factor which is expressed in a cardiogenesis region of a fetus or a factor which acts on differentiation into a cardiomyocyte in a cardiogenesis stage of a fetus.

(68) The myocardium-forming agent according to (67), wherein the factor which is expressed in a cardiogenesis region of a fetus or the factor which acts on differentiation into a cardiomyocyte in a cardiogenesis stage of a fetus is at least one selected from the group consisting of a cytokine, an adhesion molecule, a vitamin, a transcription factor, and an extracellular matrix.

(69) The myocardium-forming agent according to (68), wherein the cytokine is at least one selected from the group consisting of a platelet-derived growth factor, PDGF; a fibroblast growth factor-8, FGF-8; an endothelin 1, ET1; a midkine; and a bone morphogenetic factor, BMP-4.

(70) The myocardium-forming agent according to (69), wherein the PDGF, FGF-8, ET1, midkine, and BMP-4 comprise the amino acid sequence represented by SEQ ID NO:3 or 5, the amino acid sequence represented by SEQ ID NO:64, the amino acid sequence represented by SEQ ID NO:66, the amino acid sequence represented by SEQ ID NO:68, and the amino acid sequence represented by SEQ ID NO:70, respectively.

(71) The myocardium-forming agent according to (68), wherein the adhesion molecule is selected from the group consisting of a gelatin, a laminin, a collagen, and a fibronectin.

(72) The myocardium-forming agent according to (71), wherein the vitamin is retinoic acid.

(73) The myocardium-forming agent according to (68), wherein the transcription factor is at least one selected from the group consisting of Nkx2)5/Csx, GATA4, MEF-2A, MEF-2B, MEF-2C, MEF-2D, dHAND, eHAND, TEF-1, TEF-3, TEF-5, and MesP1.

(74) The myocardium-forming agent according to (73), wherein the Nkx2)5/Csx, GATA4, MEF-2A, MEF-2B, MEF-2C, MEF-2D, dHAND, eHAND, TEF-1, TEF-3, TEF-5, and MesP1 comprise the amino acid sequence represented by SEQ ID NO:9, the amino acid sequence represented by SEQ ID NO:11, the amino acid sequence represented by SEQ ID NO: 13, the amino acid sequence represented by SEQ ID NO:15, the amino acid sequence represented by SEQ ID NO:17, the amino acid sequence represented by SEQ ID NO:19, the amino acid sequence represented by SEQ ID NO:21, the amino acid sequence represented by SEQ ID NO:23, the amino acid sequence represented by SEQ ID NO:25, the amino acid sequence represented by SEQ ID NO:27, the amino acid sequence represented by SEQ ID NO:29, and the amino acid sequence represented by SEQ ID NO:62, respectively.

(75) The myocardium-forming agent according to (68), wherein the extracellular matrix is an extracellular matrix derived from a cardiomyocyte.

(76) A method for regenerating a heart damaged by a heart disease, comprising using the cell according to any one of (1) to (46).

(77) An agent for cardiac regeneration, comprising, as an active ingredient, the cell according to any one of (1) to (46).

(78) A method for specifically transfecting a wild-type gene corresponding to a mutant gene in a congenital genetic disease to a myocardium, comprising using the cell according to any one of (1) to (46) into which the wild-type gene corresponding to a mutant gene in a congenital genetic disease of a heart has been introduced.

(79) A therapeutic agent for a heart disease, comprising, as an active ingredient, the cell according to any one of (1) to (46) into which a wild-type gene corresponding to a mutant gene in a congenital genetic disease of a heart has been introduced.

(80) A method for producing an antibody which specifically recognizes the cell according to any one of (1) to (46), comprising using the cell as an antigen.

(81) A method for isolating a cell having the potential to differentiate into a cardiomyocyte according to any one of (1) to (46), comprising using an antibody obtained by the method according to (80).

(82) A method for obtaining a surface antigen specific for the cell according to any one of (1) to (46), comprising using the cell.

(83) A method for screening a factor which proliferates the cell according to any one of (1) to (46), comprising using the cell.

(84) A method for screening a factor which induces the cell according to any one of (1) to (46) to differentiate into a cardiomyocyte, comprising using the cell.

(85) A method for screening a factor which immortalizes the cell according to any one of (1) to (46), comprising using the cell.

(86) A method for immortalizing the cell according to any one of (1) to (46), comprising expressing a telomerase in the cell.

(87) The method according to (86), wherein the telomerase comprises the amino acid sequence represented by SEQ ID NO:31.

(88) A therapeutic agent for a heart disease, comprising, as an active ingredient, the cell according to any one of (1) to (46) which has been immortalized by expressing a telomerase.

(89) The therapeutic agent according to (88), wherein the telomerase comprises the amino acid sequence represented by SEQ ID NO:31.

(90) A culture supernatant comprising the cell according to any one of (1) to (46).

(91) A method for inducing the cell according to any one of (1) to (46) to differentiate into a cardiomyocyte, comprising using the culture supernatant according to (90).

The cells having the potential to differentiate into cardiomyocytes according to the present invention can be isolated from adult tissues such as bone marrow, muscle, brain, pancreas, liver and kidney or umbilical blood, and preferred examples include bone marrow and umbilical blood.

Any cell can be used as the pluripotent stem of the present invention, so long as it has the potential to differentiate into cardiomyocytes and other cells. Preferable examples thereof include cells having the potential to differentiate into at least cardiomyocytes, adipocytes, skeletal muscle cells and osteoblasts; cells having the potential to differentiate into at least cardiomyocyte and vascular endothelial cells; cells having the potential to differentiate into at least cardiomyocytes, adipocytes, skeletal muscle cells, osteoblasts and vascular endothelial cells; and cells having the potential to differentiate into at least cardiomyocytes, adipocytes, skeletal muscle cells, vascular endothelial cells, osteoblasts, neural cells and hepatocytes.

Also, even if cells originally have the potential to differentiate into adipocytes, skeletal muscle cells and osteoblasts but do not have the potential to differentiate into cardiomyocytes, those cells to which the potential to differentiate into cardiomyocytes can be added by the following induction method or the like, are included in the invention.

The cells of the present invention having the potential to differentiate into cardiomyocytes include cells which are CD117-positive and CD140-positive. The cells which are CD117-positive and CD140-positive preferably cells which are CD34-positive, CD117-positive and CD140-positive, and cells which are CD34-negative, CD117-positive and CD140-positive; more preferably cells which are CD144-positive, CD34-positive, CD117-positive and CD140-positive, cell which are CD144-negative, CD34-positive, CD117-positive and CD140-positive, cells which are CD144-positive, CD34-negative, CD117-positive and CD140-positive, and cells which are CD144-negative, CD34-negative, CD117-positive and CD140-positive; still more preferably cells which are CD34-positive, CD117-positive, CD14-negative, CD45-negative, CD90-negative, Flk-1-negative, CD31-negative, CD105-negative, CD144-positive, CD140-positive, CD49b-negative, CD49d-negative, CD29-positive, CD54-negative, CD102-negative, CD106-negative and CD44-positive, cells which are CD34-positive, CD117-positive, CD14-negative, CD45-negative, CD90-negative, Flk-1-negative, CD31-negative, CD105-negative, CD144-negative, CD140-positive, CD49b-negative, CD49d-negative, CD29-positive, CD54-negative, CD102-negative, CD106-negative and CD44-positive, cells which are CD34-negative, CD117-positive, CD14-negative, CD45-negative, CD90-negative, Flk-1-negative, CD31-negative, CD105-negative, CD144-positive, CD140-positive, CD49b-negative, CD49d-negative, CD29-positive, CD54-negative, CD102-negative, CD106-negative and CD44-positive, and cells which are CD34-positive, CD117-positive, CD14-negative, CD45-negative, CD90-negative, Flk-1-negative, CD31-negative, CD105-negative, CD144-negative, CD140-positive, CD49b-negative, CD49d-negative, CD29-positive, CD54-negative, CD102-negative, CD106-negative and CD44-positive. The cells which are CD117-positive and CD140-positive include mouse marrow multipotential stem cells, BMSC. Mouse bone marrow-derived pluripotent stem cells (BMSC) have been deposited on Feb. 22, 2000, in National Institute of Bioscience and Human Technology, Agency of Industrial Science and Technology (Higashi 1-1-3, Tsukuba-shi, Ibaraki, Japan) as FERM BP-7043.

Examples of the cells which originally have the potential to differentiate into adipocytes, skeletal muscle cells and osteoblasts but do not have the potential to differentiate into cardiomyocytes, to which the potential to differentiate into heart muscle cells can be added by the following induction method or the like include cells which are CD117-negative and CD140-positive, preferably cells which are CD144-negative, CD34-negative, CD117-negative and CD140-positive, more preferably cells which are CD34-negative, CD117-negative, CD14-positive, CD45-negative, CD90-negative, Flk-1-negative, CD31-negative, CD105-negative, CD144-negative, CD140-positive, CD49b-positive, CD49d-negative, CD29-positive, CD54-positive, CD102-negative, CD106-positive and CD44-positive. KUM2 cells can be exemplified as the cells which are CD34-negative, CD117-negative, CD14-positive, CD45-negative, CD90-negative, Flk-1-negative, CD31-negative, CD105-negative, CD144-negative, CD140-positive, CD49b-positive, CD49d-negative, CD29-positive, CD54-positive, CD102-negative, CD106-positive and CD44-positive.

As the species of the vital tissue or umbilical blood used in the invention, vertebrate animals, preferably warm blooded animals, and more preferably mammals such as mouse, rat, guinea pig, hamster, rabbit, cat, dog, sheep, pig, cattle, goat, monkey and human are used. Those derived from a human is preferred for human therapeutic use.

Any adult tissue or umbilical blood can be used, so long as it is derived from the above animal. In therapeutic use for the human body, it is preferred to employ those derived from humans.

Myocardial cells can be obtained by isolating cells having the potential to differentiate into cardiomyocytes from an adult tissue or umbilical blood of a mammal, such as mouse, rat or human, culturing these cells and then inducing the differentiation of cells having the potential to differentiate into cardiomyocytes.

The differentiation into not only cardiomyocytes but also vascular endothelial cells, smooth muscles, skeletal muscle cells, adipocytes, bones, cartilages, pancreatic endocrine cells, pancreatic exocrine cells, hepatocytes, glomerular cells, renal tubular cells, neurons, glial cells, oligodendrocytes, etc. can be induced using the pluripotent stem cell to obtain various cells.

Now, the present invention will be described in greater detail.

1. Isolation of Cells Having the Potential to Differentiate into Cardiomyocytes

The cells having the potential to differentiate into cardiomyocytes according to the present invention can be isolated from any tissue (for example, an adult tissue, umbilical blood), so long as cells having the potential to differentiate into cardiomyocytes can be obtained. Next, a method for isolating cells having the potential to differentiate into cardiomyocytes from bone marrow will be illustrated.

(1) Method for Isolating Bone Marrow Cells Having the Potential to Differentiate into Cardiomyocytes

The method for obtaining human cells having the potential to differentiate into cardiomyocytes from bone marrow is not particularly limited, so long as it is a safe and efficient method. For example, the method described in S. E. Haynesworth, et al., Bone, 13: 81 (1992) can be employed.

Bone marrow puncture is conducted by sternal or iliac puncture. After skin disinfection of the part for puncture, a donor is subjected to local anesthesia. Particularly, subpeiosteum is thoroughly anesthetized. The inner tube of a bone marrow puncture needle is pulled out and a 10 ml syringe containing 5000 units of heparin is attached to the needle. A required amount, normally 10-20 ml, of the bone marrow fluid is quickly taken by suction and the puncture needle is removed, followed by pressure hemostasis for about 10 minutes. The obtained bone marrow fluid is centrifuged at 1000 × g to recover bone marrow cells, which are then washed with PBS (phosphate buffered saline). After this centrifugation step is repeated twice, the obtained bone marrow cells are suspended in a cell culture medium such as A-MEM (a-modification of MEM), DMEM (Dulbecco's modified MEM) or IMDM (Isocove's modified Dulbeccos's medium) each containing 10% FBS (fetal bovine serum) to prepare a bone marrow cell suspension.

For the isolation of the bone marrow cells having the potential to differentiate into cardiomyocytes from the obtained bone marrow cell suspension, any method can be employed, so long as it is effective for removing other cells existing in the cell suspension such as hematocytes, hematopoietic stem cells, vascular stem cells and fibroblasts. For example, based on the method described in M. F. Pittenger et al., Science, 284: 143 (1999), the desired cells can be isolated by subjecting the cell suspension layered over Percoll having the density of 1.073 g/ml to centrifugation at 1100 × g for 30 minutes, and the cells on the interface are recovered. Furthermore, a bone marrow cell mixture containing the cells having the potential to differentiate into cardiomyocytes can be obtained by mixing the above cell suspension with an equal amount of Percoll solution diluted to 9/10 with 10× PBS, followed by centrifugation at 20000 × g for 30 minutes, and recovering the fraction having the density of 1.075-1.060.

The thus obtained bone marrow cell mixture containing the bone marrow cells having the potential to differentiate into cardiomyocytes is diluted into single cell using 96-well culture plates to prepare a number of clones respectively derived from single cells. The clones having the potential to differentiate into cardiomyocyte can be selected by the observation of spontaneously beating cells generated by the treatment to induce cardiomyocytes from the cells having the potential to differentiate into cardiomyocytes described below.

Rat- or mouse-derived bone marrow cells having the potential to differentiate into cardiomyocytes can be obtained, for example, in the following manner. A rat or a mouse is sacrificed by cervical dislocation and thoroughly disinfected with 70% ethanol. After the skin on the femur and quadriceps femuris are excised, the femur is put out of the knee joint with scissors and the muscle on the back side of the femur is removed. Then, the femur is put out of the hip joint with scissors and taken out. After the muscle on the femur is removed with scissors as completely as possible, the femur is cut at both ends using scissors. A needle having a size appropriate for the thickness of the bone is attached to a 2.5 ml syringe containing about 1.5 ml of a cell culture medium such as α-MEM, DMEM or IMDM each containing 10% FBS followed by injecting into the pore of femur. The needle of the syringe is put into the femur from the cut end of the knee joint side and the culture medium is injected into bone marrow, whereby bone marrow cells are pressed out of the bone from the cut end of the hip joint side. The thus obtained bone marrow cells are suspended in a culture medium by pipetting. The bone marrow cells having the potential to differentiate into cardiomyocytes can be isolated from the resulting cell suspension in the same manner as in the above isolation of the human bone marrow cells.

(2) Method for Isolating Cells having the Potential to Differentiate into Cardiomyocytes from Tissue other than Bone Marrow

According to the separation method using antibodies as described in 12 hereinafter, cells having the potential to differentiate into cardiomyocytes can be obtained form tissues other than bone marrow.

Preferred examples of the tissues other than bone marrow include umbilical blood. More specifically, it can be isolated in the following method.

First, umbilical blood is separated from the cord, followed by addition of heparin to give a final concentration of 500 units/ml. After thoroughly mixing, cells are separated from the umbilical blood by centrifugation and re-suspended in a cell culture medium, such as α-MEM (a-modified MEM), DMEM (Dulbecco's modified MEM) or IMDM (Isocove's modified Dulbecco's medium), each containing 10% FBS. From the cell suspension thus obtained, cells having the potential to differentiate into cardiomyocytes can be separated using the antibodies described below.

2. Methods for Culturing the Cells having the Potential to Differentiate into Cardiomyocytes

The cells having the potential to differentiate into cardiomyocytes isolated by the methods described in the above 1 can be usually cultured using media of known compositions (Technical Standard of Tissue Culture, Third Edition, Asakura Shoten (1996)). Preferred media are cell culture media such as α-MEM, DMEM and IMDM supplemented with a serum such as 5-20% bovine serum. Culturing can be carried out under any conditions suitable for cell culture, but is preferably carried out at a temperature of 33-37° C. in an incubator filled with 5-10% carbon dioxide gas. It is preferred to culture the cells having the potential to differentiate into cardiomyocytes in a plastic culture dish used for ordinary tissue culture so that the grown cells adhere to the dish. When cells become confluent on the dish, the medium is removed and a trypsin-EDTA solution is added to suspend the cells therein. The suspended cells may be washed with PBS or a medium for culturing the cells, diluted 5-20 times with the medium and then added to another culture dish for subculture.

3. Methods for Inducing Cardiomyocytes from Cells having the Potential to Differentiate into Cardiomyocytes

The methods for inducing cardiomyocytes from the cells having the potential to differentiate into cardiomyocytes include the following: (1) induction of differentiation by the treatment with a DNA-demethylating agent, (2) induction of differentiation using a factor which is expressed in the cardiogenesis region of a fetus or a factor which controls differentiation into cardiomyocytes in the cardiogenesis stage of a fetus, and (3) induction of differentiation using a culture supernatant of the cells having the potential to differentiate into cardiomyocytes or cardiomyocytes differentiated from the cells. Cardiomyocytes can be induced from the cells having the potential to differentiate into cardiomyocytes using such a method alone or in combination. Also, according to these methods, even mesenchymal cells which originally do not have the potential to differentiate into cardiomyocytes can be differentiated into cells having the potential to differentiate into cardiomyocytes, and cardiomyocytes can be induced.

Any DNA-demethylating agent can be used, so long as it is a compound which causes demethylation of DNA. Suitable DNA-demethylating agents include demethylase which is an enzyme which specifically removes the methylation of the cytosine residue in the GpC sequence in a chromosomal DNA, 5-azacytidine (hereinafter referred to as “5-aza-C”) and DMSO (dimethyl sulfoxide). Examples of the demethylase enzymes include demethylase having the amino acid sequence represented by SEQ ID NO:1 (Nature, 397: 579-583 (1999)). Differentiation can be induced by the treatment with a DNA-demethylating agent, for example, in the following manner.

The cells having the potential to differentiate into cardiomyocytes are cultured in the presence of 3 μmol/l to 10 μmol/l of 5-aza-C for 24 hours. After 5-aza-C is removed by replacing the culture supernatant with a fresh medium, the cells are cultured for further 2-3 weeks to obtain cardiomyocytes. The cardiomyocytes produced by culturing for 2-3 weeks are mainly sinus node cells, but culturing for more than 4 weeks induces differentiation into ventricular cardiomyocytes.

Examples of the factors which are expressed in the cardiogenesis region of a fetus and the factors which act on differentiation into cardiomyocytes in the cardiogenesis stage of a fetus include cytokines, vitamins, adhesion molecules and transcription factors.

Any cytokine can be used, so long as it stimulates the cardiomyogenic differentiation of the cells having the potential to differentiate into cardiomyocytes in the cardiogenesis stage.

The examples include platelet-derived growth factor (hereinafter referred to as “PDGF”), fibroblast growth factor 8 (FGF8), endothelin 1 (ET1), midkine, and bone morphogenic protein 4 (BMP4). Preferred examples of the PDGF include PDGF A, PDGF B, PDGF C and the like, and specific examples include those the amino acid sequences represented by SEQ ID NOS:3 and 5. Preferred examples of the FGF8, ET1, midkine, BMP4 include the amino acid sequence represented by SEQ ID NO:64, the amino acid sequence represented by SEQ ID NO:66, the amino acid sequence represented by SEQ ID NO:68, and the amino acid sequence represented by SEQ ID NO:70, respectively. The cytokine can be used, e.g., at a concentration of 10 to 40 ng/ml.

It is also possible to stimulate the cardiomyogenic differentiation of the cells having the potential to differentiate into cardiomyocytes into cardiomyocytes in the cardiogenesis stage using an inhibitor against a cytokine which suppresses the cardiomyogenic differentiation.

The cytokines which suppress the cardiomyogenic differentiation include fibroblast growth factor-2 (hereinafter referred to as “IFGF-2”), specifically, FGF-2 having the amino acid sequence represented by SEQ ID NO:7 or 8.

The inhibitors against the cytokines which suppress the cardiomyogenic differentiation include substances which inhibit the signal transduction of the cytokines, such as antibodies and low molecular weight compounds which neutralize the cytokines activities.

Any vitamin can be used, so long as it stimulates the cardiomyogenic differentiation of the cells having the potential to differentiate into cardiomyocytes in the cardiogenesis stage. Retinoic acid can be used, e.g., at a concentration of 10⁻⁹ M.

Any adhesion molecule can be used, so long as it is expressed in the cardiogenesis region in the cardiogenesis stage. Examples include extracellular matrices such as gelatin, laminin, collagen, fibronectin and the like. For example, the cardiomyogenic differentiation of the cells having the potential to differentiate into cardiomyocytes can be stimulated by culturing the cells on a culture dish coated with fibronectin.

Examples of the transcription factors include a homeobox-type transcription factor, Nkx2.5/Csx (SEQ ID NO:9, amino acid sequence; SEQ ID NO:10, nucleotide sequence); a zinc finger-type transcription factor belonging to the GATA family, GATA4 (SEQ ID NO:11, amino acid sequence; SEQ ID NO:12, nucleotide sequence); transcription factors belonging to the myocyte enhance factor-2 (MEF-2) family, MEF-2A (SEQ ID NO:13, amino acid sequence; SEQ ID NO:14, nucleotide sequence), MEF-2B (SEQ ID NO:15, amino acid sequence; SEQ ID NO:16, nucleotide sequence), MEF-2C (SEQ ID NO:17, amino acid sequence; SEQ ID NO:18, nucleotide sequence) and MEF-2D (SEQ ID NO:19, amino acid sequence; SEQ ID NO:20, nucleotide sequence); transcription factors belonging to the basic helix loop helix-type transcription factors, dHAND (SEQ ID NO:21, amino acid sequence; SEQ ID NO:22, nucleotide sequence) and eHAND (SEQ ID NO:23, amino acid sequence; SEQ ID NO:24, nucleotide sequence); and transcription factors belonging to the family of TEA-DNA binding-type transcription factors, TEF-1 (SEQ ID NO:25, amino acid sequence; SEQ ID NO:26, nucleotide sequence), TEF-3 (SEQ ID NO:27, amino acid sequence; SEQ ID NO:28, nucleotide sequence) and TEF-5 (SEQ ID NO:29, amino acid sequence; SEQ ID NO:30, nucleotide sequence).

The cardiomyogenic differentiation of the cells having the potential to differentiate into cardiomyocytes can be induced by introducing DNA encoding one or combination of the above-described factors into the cells and expressing the DNA therein.

It is also possible to induce the cardiomyogenic differentiation of the cells having the potential to differentiate into cardiomyocytes by culturing them using a culture dish coated with an extracellular matrix obtained from spontaneously beating cardiomyocytes, co-culturing with spontaneously beating cardiomyocytes or adding a culture supernatant of spontaneously beating cardiomyocytes.

Furthermore, a factor which induces differentiation of cardiomyocytes which are obtained by the method described in 4 below (hereinafter referred to as “the cardiomyogenic differentiation-inducing factor”) can also be used in inducing the cardiomyogenic differentiation of the cells having the potential to differentiate into cardiomyocytes.

4. Methods for Obtaining Cardiomyogenic Differentiation-Inducing Factors

A cardiomyogenic differentiation-inducing factor can be obtained by adding various protease inhibitors to a culture supernatant of spontaneously beating cardiomyocytes, followed by combinations of treatments, such as dialysis, salting-out and chromatography.

Genes encoding such cardiomyogenic differentiation-inducing factors can be obtained by determining partial amino acid sequences of these factors using a microsequencer followed by screening a cDNA library prepared from the spontaneously beating cells using DNA probes designed based on the determined amino acid sequences.

5. Therapeutic Agents for Cardiac Regeneration and Therapeutic Agents for Heart Diseases Comprising Cells having the Potential to Differentiate into Cardiomyocytes

The cells having the potential to differentiate into cardiomyocytes according to the present invention can be used as therapeutic agents for cardiac regeneration or for heart diseases.

The heart diseases include myocardial infarction, ischemic heart disease, congestive heart failure, arrhythmia, hypertrophic cardiomyopathy, dilated cardiomyopathy, myocarditis and valvular disease.

The agents for cardiac regeneration contain the cells having the potential to differentiate into cardiomyocytes of high purity which cells have been proliferated in vitro according to the position and size of the damaged part of the heart. The preferred cells having the potential to differentiate into cardiomyocytes are those which can be induced to differentiate into various cells constituting the heart such as endocardial endothelial cells, cushion cells, ventricular cardiomyocytes, atrial cardiomyocytes and sinus node cells.

The therapeutic agents can be prepared by purifying the cells having the potential to differentiate into cardiomyocytes from the bone marrow fluid taken from myocardial infarction patients according to the above-described density gradient centrifugation, the panning method (J. Immunol., 141(8): 2797-800 (1988)) or the FACS method (Int. Immunol., 275-83 (1998)) using the antibodies described below which specifically recognize the cells having the potential to differentiate into cardiomyocytes, or a method for constructing a reporter system using the promoter of a gene specifically expressed in the cell having the potential to differentiate into cardiomyocytes.

The therapeutic agents include cardiomyocytes derived from the cells having the potential to differentiate into cardiomyocytes using the myocardium-forming agent described below as well as the cells having the potential to differentiate into cardiomyocytes which are obtained by activating the division potential of the bone marrow cells taken from the bone marrow of aged persons by utilizing the immortalization method described below.

The purity of the therapeutic agents prepared according to the above methods can be tested by the FACS method combined with the antibodies which specifically recognize the cells having the potential to differentiate into cardiomyocytes.

The therapeutic agents can be transported to the damaged parts by a method using a catheter or the like. For example, in the case of ischemic heart disease, the therapeutic agents are transported according to the following procedure. Since the cardiomyocytes damaged by ischemic heart disease exist downstream of vascular stricture, it is necessary to locate the vascular stricture by coronary arteriography (Illustrated Pathological Internal Medical Course Circulateory Organ, 1, MEDICAL VIEW, 1993) prior to the injection of the above cells. Organic stricture is classified as concentric stricture, eccentric stricture or multiple mural asymmetry according to type of stricture, and eccentric stricture is further classified into two types, i.e. type I and type II. It is known that the types of stricture are related to the course and prognosis of angina; for instance, eccentric stricture of type II and multiple mural asymmetry are often observed in unstable angina which is liable to shift into myocardial infarction. In cases where blood vessels are completely strictured, there is the possibility that the injected cells can not reach the damaged parts. In such cases, the strictured parts must be reopened by means of percutaneous transluminal coronary angioplasty (PTCA), thrombolytic treatment or the like prior to the cell injection. The type of the cells to be injected such as ventricular or atrial can be selected according to the position of the damaged cardiomyocytes. The insertion of a catheter can be performed by the Sones method (Illustrated Pathological Internal Medical Course Circulateory Organ, 1, MEDICAL VIEW, 1993) through the artery of the right upper arm or by the Jundkins method (Illustrated Pathological Internal Medical Course Circulateory Organ, 1, MEDICAL VIEW, 1993) through the femural artery.

6. Myocardium-Forming Agents

The myocardium-forming agents according to the present invention comprise, as an active ingredient, at least one cardiomyogenic differentiation-inducing factor selected from the group consisting of a chromosomal DNA-demethylating agent, a factor which is expressed in the cardiogenesis region of a fetus, and a factor which acts on differentiation into cardiomyocytes in the cardiogenesis stage of a fetus, and are capable of inducing the bone marrow-derived cells to differentiate into cardiomyocytes.

Examples of the cardiomyogenic differentiation-inducing factors include cytokines, vitamins, adhesion molecules and transcription factors.

Any cytokine can be used, so long as it stimulates the cardiomyogenic differentiation of the cells having the potential to differentiate into cardiomyocytes in the cardiogenesis stage.

For example, PDGF, FGF-8, endotherin 1 (ET1), Midkine and Bone Marrow Protein 4 (BMP4) can be used. Preferable examples of the PDGF, FGF8, ET1, Midkine, BMP4 include those the amino acid sequences represented by SEQ ID NOS:3 and 5, the amino acid sequence represented by SEQ ID NO:64, the amino acid sequence represented by SEQ ID NO:66, the amino acid sequence represented by SEQ ID NO:68, and the amino acid sequence represented by SEQ ID NO:70, respectively. The cytokine can be used, e.g., at a concentration of 10 to 40 ng/ml.

Any vitamin can be used, so long as it stimulates the cardiomyogenic differentiation of the cells having the potential to differentiate into cardiomyocytes in the cardiogenesis stage. Retinoic acid can be used, e.g., at a concentration of 10⁻⁹ M.

Any adhesion molecule can be used so far as it is expressed in the cardiogenesis region in the cardiogenesis stage. Examples include gelatin, laminin, collagen, fibronectin and the like. For example, the cardiomyogenic differentiation of the cells having the potential to differentiate into cardiomyocytes can be stimulated by culturing the cells in a culture dish coated with fibronectin.

Examples of the transcription factors include a homeobox-type transcription factor, Nkx2.5/Csx (SEQ ID NO:9, amino acid sequence; SEQ ID NO:10, nucleotide sequence); a zinc finger-type transcription factor belonging to the GATA family, GATA4 (SEQ ID NO:11, amino acid sequence; SEQ ID NO:12, nucleotide sequence); transcription factors belonging to the myocyte enhancer factor-2 (MEF-2) family, MEF-2A (SEQ ID NO:13, amino acid sequence; SEQ ID NO:14, nucleotide sequence), MEF-2B (SEQ ID NO:15, amino acid sequence; SEQ ID NO:16, nucleotide acid sequence), MEF-2C (SEQ ID NO:17, amino acid sequence; SEQ ID NO:18, nucleotide sequence) and MED-2D (SEQ ID NO:19, amino acid sequence; SEQ ID NO:20, nucleotide sequence); transcription factors belonging to the basic helix loop helix-type transcription factors, dHAND (SEQ ID NO:21, amino acid sequence; SEQ ID NO:22, nucleotide sequence), eHAND (SEQ ID NO:23, amino acid sequence; SEQ ID NO:24, nucleotide sequence) and MesP1 (SEQ ID NO:61, amino acid sequence; SEQ ID NO:62, nucleotide sequence); and transcription factors belonging to the family of TEA-DNA binding-type transcription factors, TEF-1 (SEQ ID NO:25, amino acid sequence; SEQ ID NO:26, nucleotide sequence), TEF-3 (SEQ ID NO:27, amino acid sequence; SEQ ID NO:28, nucleotide sequence) and TEF-5 (SEQ ID NO:29, amino acid sequence; SEQ ID NO:30, nucleotide sequence).

The myocardium-forming agents can contain, as a main component, either a gene encoding a cardiomyogenic differentiation-inducing factor or a protein which is a cardiomyogenic differentiation-inducing factor itself.

(1) Myocardium-Forming Agent Containing Gene as Main Component

Methods for preparing the myocardium-forming agents of the present invention which comprise, as a main component, a gene encoding a cardiomyogenic differentiation-inducing factor are described below.

First, a DNA fragment or the full length cDNA of a gene encoding a cardiomyogenic differentiation-inducing factor is inserted downstream of a promoter in a virus vector plasmid to construct a recombinant virus vector plasmid.

Then, the obtained recombinant virus vector plasmid is introduced into a packaging cell which is suitable for the virus vector plasmid.

The recombinant virus vector plasmid lacks at least one of the genes encoding the proteins necessary for the packaging of a virus. As the packaging cell, any cell can be used so far as it can supply the protein encoded by the lacking gene. Suitable packaging cells include HEK293 cell derived from human kidney and mouse fibroblast NIH3T3.

Examples of the proteins supplied by the packaging cells include proteins, such as gag, pol and env, derived from mouse retroviruses for retrovirus vectors; proteins, such as gag, pol, env, vpr, vpu, vif, tat, rev and nef, derived from HIV viruses for lentivirus vectors; proteins, such as E1A and E1B, derived from adenoviruses for adenovirus vectors; and proteins, such as Rep(p5, p19, p40) and Vp(Cap), for adeno-associated viruses.

The virus vector plasmids that can be employed are those capable of producing a recombinant virus in the above packaging cells and comprising a promoter at a position appropriate for the transcription of a wild-type gene corresponding to the causative gene of a congenital genetic heart disease in cardiomyocytes.

Suitable virus vector plasmids include MFG (Proc. Natl. Acad. Sci. USA, 92: 6733-6737 (1995)), pBabePuro (Nucleic Acids Research, 18: 3587-3596 (1990)), LL-CG, CL-CG, CS-CG and CLG (Journal of Virology, 72: 8150-8157 (1998)) and pAdex1 (Nucleic Acids Res., 23: 3816-3812 (1995)).

Any promoter can be used as long as it can be expressed in human tissues. Examples of suitable promoters are the promoter of IE (immediate early) gene of cytomegalovirus (human CMV), SV40 early promoter, the promoter of a retrovirus, metallothionein promoter, heat shock protein promoter and SRα promoter. The enhancer of IE gene of human CMV may be used in combination with the promoter. It is possible to express the desired gene specifically in cardiomyocytes using a promoter of a gene specifically expressed in cardiomyocytes such as Nkx2.5/Csx gene.

A recombinant virus vector can be produced by introducing the above recombinant virus vector plasmid into the above packaging cell. Introduction of the virus vector plasmid into the packaging cell can be carried out, for example, by the calcium phosphate method (Japanese Published Unexamined Patent Application No. 227075/90) or the lipofection method (Proc. Natl. Acad. Sci. USA, 84: 7413 (1987)).

The above recombinant virus vector can be formulated into myocardium-forming agents by admixture with a carrier used in pharmaceutical compositions for gene therapy (Nature Genet., 8: 42 (1994)). Any carrier can be used so long as it is usually used in injections. Suitable carriers include distilled water, salt solutions of sodium chloride or mixtures of sodium chloride and inorganic salts, solutions of mannitol, lactose, dextran, glucose, etc., solutions of amino acids such as glycine and arginine, and mixtures of organic acid solutions or salt solutions and a glucose solution. Injections may be prepared in the form of solutions, suspensions or dispersed solutions according to conventional methods using the above carriers as well as auxiliaries, for example, osmotic pressure adjusting agents, pH adjusting agents, vegetable oils such as sesame oil and soybean oil, lecithin, and surfactants such as nonionic surfactants. If desired, the injections may be prepared in the form of powdered or freeze-dried preparations which are dissolved in a solvent before each use. The myocardium-forming agents in the form of liquid preparations can be used as such for gene therapy, and those in the form of solid preparations are dissolved, immediately before use, in the above carriers which are sterilized if necessary. Administration of the myocardium-forming agents is made locally using a catheter or the like so that the agents can be absorbed into the myocardium of a patient.

The cells having the potential to differentiate into cardiomyocytes infected with the above recombinant virus vector in vitro can also be formulated into the above myocardium-forming agents and administered to a patient. Furthermore, the recombinant virus vector can be directly administered to the diseased part of a patient.

(2) Myocardium-Forming Agent Containing Protein as Main Component

Methods for preparing the myocardium-forming agents of the present invention which contains as a main component, a protein which is a cardiomyogenic differentiation-inducing factor are described, below.

On the basis of the full length CDNA encoding a cardiomyogenic differentiation-inducing factor, if necessary, a DNA fragment having an appropriate length containing a region encoding the protein is prepared.

The prepared DNA fragment or the full length cDNA is inserted downstream of a promoter in an expression vector to construct a recombinant expression vector for the protein.

Then, the recombinant expression vector is introduced into a host cell suited for the expression vector.

Any cell can be used so long as it is capable of expressing the desired gene products. Examples of the host cells include bacteria belonging to the genus Escherichia, the genus Serratia, the genus Corynebacterium, the genus Brevibacterium, the genus Pseudomonas, the genus Bacillus and the genus Microbacterium, yeasts belonging to the genus Kluyveromyces, the genus Saccharomyces, the genus Shizosaccharomyces, the genus Trichosporon and the genus Schwanniomyces, animal cells and insect cells.

The expression vectors that can be employed are those capable of autonomous replication or integration into chromosome in the above host cells and containing a promoter at a position suitable for the transcription of a gene of a cardiomyogenic differentiation-inducing factor.

When bacteria are used as the host cell, it is preferred that the recombinant expression vector for a gene encoding a cardiomyogenic differentiation-inducing factor is a recombinant vector which is capable of autonomous replication in the bacterial cell and which comprises a promoter, a ribosome binding sequence, a DNA encoding a protein which can induce cardiomyogenic differentiation, and a transcription termination sequence. The vector can further comprise a gene regulating the promoter.

Examples of suitable expression vectors include pBTrp2, pBTac1 and pBTac2 (manufactured by Boehringer Mannheim), pKK233-2 (manufactured by Amersham Pharmacia Biotech), pSE280 (manufactured by Invitrogen), pGEMEX-1 (manufactured by Promega), pQE-8 (manufactured by QIAGEN), pKYP10 (Japanese Published Unexamined Patent Application No. 110600/83), pKYP200 (Agricultural Biological Chemistry, 48: 669 (1984)), pLSA1 (Agric. Biol. Chem., 53: 277 (1989)), pGEL1 (Proc. Natl. Acad. Sci. USA, 82: 4306 (1985)), pBluescript II SK (−) (manufactured by Stratagene), pGEX (manufactured by Amersham Pharmacia Biotech), pET-3 (manufactured by Novagen), pTerm2 (U.S. Pat. Nos. 4,686,191, 4,939,094 and 5,160,735), and pSupex, pUB110, pTP5, pC194 and pEG400 (J. Bacteriol., 172: 2392 (1990)).

It is preferred to use a plasmid in which the distance between the Shine-Dalgarno sequence (ribosome binding sequence) and the initiation codon is adjusted to a suitable length (e.g., 6-18 bases).

Any promoter can be used so long as it can be expressed in the host cell. For example, promoters derived from Escherichia coli or a phage, such as trp promoter (Pₜᵣₚ), lac promoter (P_{lac}), P_{L} promoter, P_{R} promoter and T7 promoter, SPO1 promoter, SPO2 promoter and penP promoter can be used. Artificially modified promoters such as a promoter in which two Ptrp are combined in tandem (Pₜᵣₚ×2), tac promoter, letI promoter (Gene, 44: 29 (1986)) and lacT7 promoter can also be used.

The yield of the desired protein can be improved by replacing a nucleotide in the nucleotide sequence of the protein-encoding region in the gene of the cardiomyogenic differentiation-inducing factor of the present invention so as to make a codon most suitable for the expression in a host cell.

The transcription termination sequence is not essential for the expression of the gene encoding the cardiomyogenic differentiation-inducing factor of the present invention, but it is preferred that the transcription termination sequence is located immediately downstream of the structural gene.

Examples of suitable host cells are cells of microorganisms belonging to the genus Escherichia, the genus Serratia, the genus Corynebacterium, the genus Brevibacterium, the genus Pseudomonas, the genus Bacillus and the genus Microbacterium, specifically, Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Bacillus subtilis, Bacillus amyloliquefaciens, Brevibacterium ammoniagenes, Brevibacterium immariophilum ATCC 14068, Brevibacterium saccharolyticum ATCC 14066, Corynebacterium glutamicum ATCC 13032, Corynebacterium glutamicum ATCC 14067, Corynebacterium glutamicum ATCC 13869, Corynebacterium acetoacidophilum ATCC 13870, Microbacterium ammmoniaphilum ATCC 15354 and Pseudomonas sp. D-0110.

Introduction of the recombinant vector can be carried out by any of the methods for introducing DNA into the above host cells, for example, the method using calcium ion (Proc. Natl. Acad. Sci. USA, 69: 2110 (1972)), the protoplast method (Japanese Published Unexamined Patent Application No. 248394/88) and the methods described in Gene, 17: 107 (1982) and Molecular & General Genetics, 168: 111 (1979).

When yeast is used as the host cell, YEp13 (ATCC 37115), YEp24 (ATCC 37051), YCp50 (ATCC 37419), pHS19, pHS15, etc. can be used as the expression vector.

Any promoter can be used, so long as it can be expressed in the yeast. Suitable promoters include PH05 promoter, PGK promoter, GAP promoter, ADH promoter, gal 1 promoter, gal 10 promoter, heat shock protein promoter, MFα 1 promoter and CUP 1 promoter.

Examples of suitable host cells include cells of Saccharomyces cerevisiae, Schizosaccharomyces pombe, Kluyveromyces lactis, Trichosporon pullulans and Schwanniomyces alluvius.

Introduction of the recombinant vector can be carried out by any of the methods for introducing DNA into yeast cells, for example, electroporation (Methods. Enzymol, 194: 182 (1990)), the spheroplast method (Proc. Natl. Acad. Sci. USA, 75: 1929 (1978)) and the lithium acetate method (J. Bacteriol., 153: 163 (1983), Proc. Natl. Acad. Sci. USA, 75: 1929 (1978)).

When an animal cell is used as the host cell, pcDNAI (manufactured by Invitorogen), pcDM8 (manufactured by Invitorogen), pAGE107 (Japanese Published Unexamined Patent Application No. 22979/91, Cytotechnology, 3: 133 (1990)), pAS3-3 (Japanese Published Unexamined Patent Application No. 227075/90), pCDM8 (Nature, 329: 840 (1987)), pcDNAI/Amp (manufactured by Invitrogen), pREP4 (manufactured by Invitrogen), pAGE103 (J. Biochem. 101: 1307 (1987)), pAGE210, etc. can be used as the expression vector.

As the promoter, any promoters capable of expression in animal cells can be used. Suitable promoters include the promoter of IE (immediate early) gene of cytomegalovinus (human CMV), SV40 early promoter, the promoter of a retrovirus, metallothionein promoter, heat shock protein promoter and SRα promoter. The enhancer of IE gene of human CMV may be used in combination with the promoter.

Examples of suitable host cells are human Namalwa cell, monkey COS cell, Chinese hamster CHO cell and HBT5637 (Japanese Published Unexamined Patent Application No. 299/88).

Introduction of the recombinant vector can be carried out by any of the methods for introducing DNA into animal cells, for example, electroporation method (Cytotechnology, 3: 133 (1990)), the calcium phosphate method (Japanese Published Unexamined Patent Application No. 227075/90), and lipofection method (Proc. Natl. Acad. Sci. USA, 84: 7413 (1987), Virology, 52: 456 (1973)). A transformant can be obtained and cultured according to the methods described in Japanese Published Unexamined Patent Application Nos. 227075/90 and 257891/90.

When an insect cell is used as the host cell, the protein can be expressed using the methods descried in Baculovirus Expression Vectors, A Laboratory Manual, W. H. Freeman and Company, New York (1992), Current Protocols in Molecular Biology, Supplement 1-38 (1987-1997), Bio/Technology, 6: 47 (1988), etc.

Specifically, the recombinant gene transfection vector and a baculovirus are cotransfected into an insect cell to obtain a recombinant virus in the culture supernatant of the insect cell, and then an insect cell is infected with the recombinant virus to express the protein.

Examples of the gene transfection vectors suitable for use in this method are pVL1392, pVL1393 and pBlueBacIII (manufactured by Invitrogen).

Examples of the baculovirus include Autographa californica nuclear polyhedrosis virus with which an insect belonging to the family Barathra is infected.

Examples of the insect cells include Sf9 and Sf21 (Baculovirus Expression Vectors, A Laboratory Manual, W. H. Freeman and Company, New York (1992)), which are ovary cells of Spodoptera frugiperda, and High 5 (manufactured by Invitrogen), which is an ovary cell of Trichoplusia ni.

Cotransfection of the recombinant gene transfection vector and the baculovirus into an insect cell for the preparation of the recombinant virus can be carried out by the calcium phosphate method (Japanese Published Unexamined Patent Application No. 227075/90), the lipofection method (Proc. Natl. Acad. Sci. USA, 84: 7413 (1987)), etc.

Expression of the gene can be carried out not only by direct expression but also by secretory production, fused protein expression, etc. according to the methods described in Molecular Cloning, A Laboratory Manual, Second Edition, Cold Spring Harbor Laboratory Press (1989) (hereinafter referred to as “Molecular Cloning, A Laboratory Manual, 2nd ed.”) etc.

When the gene is expressed in yeast, an animal cell or an insect cell, a glycoprotein or glycosylated protein can be obtained.

The protein as the cardiomyogenic differentiation-inducing factor can be produced by culturing the transformant carrying the recombinant DNA containing the DNA encoding the protein as the cardiomyogenic differentiation-inducing factor in a medium, allowing the protein to accumulate in the culture, and recovering the protein from the culture.

Culturing of the transformant for the production of the protein as the cardiomyogenic differentiation-inducing faactor can be carried out by conventional methods for culturing the host cell of the transformant.

For the culturing of the transformant prepared using a procaryotic cell such as E. coli or a eucaryotic cell such as yeast as the host cell, any of natural media and synthetic media can be used, so long as it is a medium suitable for efficient culturing of the transformant which contains a carbon source, a nitrogen source, an inorganic substance, etc. which can be assimilated by the host used.

Any carbon source can be used, so long as it can be assimilated by the host. Examples of suitable carbon sources include carbohydrates such as glucose, fructose, sucrose, molasses containing them, starch and starch hydrolyzate; organic acids such as acetic acid and propionic acid; and alcohols such as ethanol and propanol.

Examples of the nitrogen sources include ammonia, ammonium salts of inorganic or organic acids such as ammonium chloride, ammonium sulfate, ammonium acetate and ammonium phosphate, and other nitrogen-containing compounds can be used as well as peptone, meat extract, yeast extract, corn steep liquor, casein hydrolyzate, soybean cake, soybean cake hydrolyzate, and various fermented cells and digested products thereof.

Examples of the inorganic substances include potassium dihydorgenphosphate, dipotassium hydrogenphosphate, magnesium phosphate, magnesium sulfate, sodium chloride, ferrous sulfate, manganese sulfate, copper sulfate and calcium carbonate.

Culturing is usually carried out under aerobic conditions, for example, by shaking culture or submerged spinner culture under aeration, at 15-40° C. for 16 hours to 7 days. The pH is maintained at 3.0-9.0 during the culturing. The pH is adjusted using an organic or inorganic acid, an alkali solution, urea, calcium carbonate, ammonia, etc.

If necessary, antibiotics, such as ampicillin and tetracycline, can be added to the medium during the culturing.

When a microorganism transformed with an expression vector comprising an inducible promoter is cultured, an inducer may be added to the medium, if necessary. For example, in the case of a microorganism transformed with an expression vector containing lac promoter, isopropyl-β-D-thiogalactopyranoside (IPTG) or the like can be added to the medium; and in the case of a microorganism transformed with an expression vector containing trp promoter, indoleacrylic acid (IAA) or the like can be added.

For the culturing of the transformant prepared using an animal cell as the host cell, generally used media such as RPMI1640 medium (The Journal of the American Medical Association, 199: 519 (1967)), Eagles's MEM (Science, 122: 501 (1952)), Dulbecco's modified MEM (Virology, 8: 396 (1959)) and 199 medium (Proceeding of the Society for the Biological Medicine, 73: 1 (1950)), media prepared by adding fetal calf serum to these media, etc. can be used as the medium.

Culturing is usually carried out at pH 6-8 at 30-40° C. for 1-7 days in the presence of 5% Co₂.

If necessary, antibiotics, such as kanamycin and penicillin, can be added to the medium during the culturing.

For the culturing of the transformant prepared using an insect cell as the host cell, generally used media such as TNM-FH medium (manufactured by Pharmingen), Sf-900II SFM medium (manufactured by Life Technologies), ExCell 400 and ExCell 405 (manufactured by JRH Biosciences) and Grace's Insect Medium (Grace, T. C. C., Nature, 195: 788 (1962)) can be used as the medium.

Culturing is usually carried out at pH 6-7 at 25-30° C. for 1-5 days.

If necessary, antibiotics, such as gentamicin, can be added to the medium during the culturing.

The protein as the cardiomyogenic differentiation-inducing factor can be isolated and purified from the culture of the transformant by conventional methods for isolating and purifying proteins.

For example, when the protein as the cardiomyogenic differentiation-inducing factor is expressed in a soluble form in cells, the isolation and purification can be carried out in the following manner. After the completion of culturing, the cells are recovered from the culture by centrifugation and suspended in an aqueous buffer, followed by disruption using an ultrasonic disrupter, a French press, a Manton Gaulin homogenizer, a Dyno Mill, etc. to obtain a cell-free extract. The cell-free extract is centrifuged, and a purified protein preparation can be produced from the obtained supernatant using ordinary means for isolation and purification of proteins, for example, extraction with a solvent, salting-out with ammonium sulfate, etc., desalting, precipitation with an organic solvent, anion exchange chromatography using resins such as diethylaminoethyl (DEAE)-Sepharose and DIAION HPA-75 (Mitsubishi Chemical Corporation), cation exchange chromatography using resins such as S-Sepharose FF (manufactured by Amersham Pharmacia Biotech), hydrophobic chromatography using resins such as butyl Sepharose and phenyl Sepharose, gel filtration using a molecular sieve, affinity chromatography, chromatofocusing, and electrophoresis such as isoelectric focusing, alone or in combination.

When the protein is expressed as an insoluble substance in cells, the cells are separated and disrupted, followed by centrifugation to recover the insoluble substance of the protein as a precipitate fraction.

The recovered insoluble substance of the protein is solubilized with a protein-denaturing agent. The solubilized protein solution is diluted or dialyzed to lower the concentration of the protein-denaturing agent therein, thereby restoring the normal tertiary structure of the protein, followed by the same isolation and purification steps as described above to obtain a purified protein preparation.

When the protein as the cardiomyogenic differentiation-inducing factor or its derivatives, such as a glycosylated protein, are extracellularly secreted, they can be recovered from the culture supernatant. That is, the culture is treated by means such as centrifugation and the obtained culture supernatant is subjected to the same isolation and purification steps as mentioned above to obtain a purified protein preparation.

The thus obtained proteins include the proteins having the amino acid sequences represented by SEQ ID NOS:5, 6, 10, 12, 14, 16, 18, 20, 22, 24, 26, 28 and 30.

The proteins expressed by the above methods can also be produced by chemical synthetic methods such as the Fmoc method (the fluorenylmethyloxycarbonyl method) and the tBoc method (the t-butyloxycarbonyl method). Furthermore, the proteins can be synthesized using peptide synthesizers (for example, manufactured by Advanced ChemTech, Perkin-Elmer, Amersham Pharmacia Biotech, Protein Technology Instrument, Synthecell-Vega, PerSeptive, Shimadzu Corporation, etc.).

The protein which can induce cardiomyogenic differentiation can be formulated into myocardium-forming agents and administered in the same manner as in the above (1).

7. Application to Therapy of Congenital Genetic Disease

In some of the diseases leading to heart failure, the deficiency of an essential protein due to the mutation of a single gene causes heart failure. Examples of such diseases are familial hypertrophic cardiomyopathy, Fabri disease, QT elongation syndrome, Marfan syndrome, aortic stenosis, mitochondria cardiomyopathy and Duchenne muscular dystrophy. These diseases are known to be caused by the abnormality in the genes of myosin, troponin, tropomyosin, potential-dependent Na channel, K channel, fibrin, elastin, mitochondria, dystrophin, etc. (Therapeutics, 30: 1302-1306 (1996)).

The method for treating a patient of the above disease includes a method comprising acquiring the cells having the potential to differentiate into cardiomyocytes of the present invention from a patient of the disease, introducing the wild type gene corresponding to the causative gene of the disease into the cells, and transplanting the cells to the patient's heart. The normal gene is inserted into the vector for gene therapy described in the above 6(1), and then can be introduced into the cells having the potential to differentiate into cardiomyocytes of the present invention using the vector for gene therapy described in the above 6(1).

8. Methods for Obtaining Antibody which Specifically Recognizes Surface Antigen Specific for Cells having the Potential to Differentiate into Cardiomyocytes

Methods for preparing antibodies which specifically recognize surface antigens expressed in the cells having the potential to differentiate into cardiomyocytes of the present invention are described below.

The antibodies which recognize the surface antigens expressed specifically in the cells having the potential to differentiate into cardiomyocytes of the present invention are useful in the purity test and purification of the cells required for applying the cells to the therapy of heart diseases such as myocardial infarction.

In order to obtain the antibody, an antigen is administered subcutaneously, intravenously or intraperitoneally to a non-human mammal, such as rabbit or goat, or 3 to 20-weeks-old rat, mouse or hamster together with an appropriate adjuvant, such as complete Freund's adjuvant, aluminum hydroxide gel or pertussis vaccine. As the antigen, the cells having the potential to differentiate into cardiomyocytes of the present invention (3×10⁵ to 5×10⁵ cells/animal) or the cell membrane fraction prepared from the cells (1-10 mg/animal) is used.

Administration of the antigen is repeated 3 to 10 times after the first administration at intervals of 1 to 2 weeks. On the 3rd to 7th day after each administration, a blood sample is collected from fundus oculi veniplex and the obtained serum is examined from reactivity to the antigen used for immunization according to enzyme immunoassay (Enzyme-Linked Immuno Adsorbent Assay (ELISA), Igaku Shoin (1976), Antibodies—A Laboratory Manual, Cold Spring Harbor Laboratory (1988)). A non-human mammal whose serum shows a sufficient antibody titer against the antigen used for immunization is employed as a source of serum or antibody-producing cell.

The polyclonal antibody can be prepared by separation and purification from the serum.

For the preparation of the monoclonal antibody, the antibody-producing cell and a myeloma cell derived from a non-human mammal are fused to obtain hybridoma, and the hybridoma is cultured or administered to an animal to cause ascites tumor. The monoclonal antibody can be prepared by separation and purification from the resulting culture or ascites.

Examples of the antibody-producing cells include spleen cells and antibody-producing cells in lymph nodes or peripheral blood, and among these, spleen cells are preferably used.

As the myeloma cells, mouse-derived cell lines are preferably used. Examples of suitable cell lines are P3-X63Ag8-U1 (P3-U1) cell line (Current Topics in Microbiology and Immunology, 18: 1 (1978)), which is 8-azaguanine-resistant mouse (BALB/c-derived) myeloma cell line, P3-NS1/1-Ag41(NS-1) line (European J. Immunology, 6: 511 (1976)), SP2/0-Ag14(SP-2) line (Nature, 276: 269 (1978)), P3-X63-Ag8653(653) line (J. Immunology, 123: 1548 (1979)) and P3-X63-Ag8(X63) line (Nature, 256: 495 (1975)).

The hybridoma can be prepared in the following manner.

The antibody-producing cells and the myeloma cells are mixed and suspended in HAT medium (a medium prepared by adding hypoxanthine, thymidine and aminopterin to a normal medium), followed by culturing for 7-14 days. After the culturing, a portion of the culture supernatant is subjected to enzyme immunoassay to select cells which react with the antigen and do not react with the protein containing no antigen. Then, cloning is carried out by limiting dilution method, and cells showing a high and stable antibody titer according to enzyme immunoassay are selected as the monoclonal antibody-forming hybridomas.

Separation and purification of the polyclonal antibodies and the monoclonal antibodies can be carried out using means such as centrifugation, ammonium sulfate precipitation, caprylic acid precipitation, and chromatography using DEAE-Sepharose column, anion exchange column, protein A- or G-column or gel filtration column, alone or in combination.

Sampling cells can be easily tested for expression of the surface antigen expressed in the cells having the potential to differentiate into cardiomyocytes by comparing the reactivity of the thus obtained antibody specifically which recognizes the surface antigen to the test cells with that to control cells such as hematopoietic stem cells and neural stem cells.

9. Methods for Obtaining Surface Antigen Expressed in Cells having the Potential to Differentiate into Cardiomyocytes and gene Encoding the Surface Antigen

The genes encoding the surface antigens expressed specifically in the cells having the potential to differentiate into cardiomyocytes can be obtained by the cDNA subtraction method (Proc. Natl. Acad. Sci. USA, 85: 5738-5742 (1988)) and the representational difference analysis (Nucleic Acids Research, 22: 5640-5648 (1994)), which are methods for obtaining genes showing different expression profiles between two samples of different origins.

First, a cDNA library prepared from the cells having the potential to differentiate into cardiomyocytes is subjected to subtraction using mRNA obtained from control cells other than cells having the potential to differentiate into cardiomyocytes, e.g., hematopoietic stem cells and neural stem cells. Then a subtracted cDNA library with a high content of a gene specifically expressed in the cells having the potential to differentiate into cardiomyocytes is prepared, followed by nucleotide sequence analysis of inserted cDNA in the subtracted cDNA library from the 5′ terminal side randomly to select those having the secretion signal sequence (random sequence analysis). The full length nucleotide sequences of the thus obtained cDNAs are determined to distinguish the proteins encoded by the cDNAs into secretory proteins and membrane proteins.

In the above process, the signal sequence trap method can be used instead of the random sequence analysis (Science, 261: 600-603 (1993), Nature Biotechnology, 17: 487-490 (1999)). The signal sequence trap method is a method for selectively screening for genes having the secretion signal sequence.

In order to efficiently obtain the specific surface antigens, it is preferred to prepare a signal sequence trap library from the cells having the potential to differentiate into cardiomyocytes using a vector suitable for subtraction and to subject the signal sequence trap library to subtraction using mRNA obtained from control cells such as hematopoietic stem cells and neural stem cells. The thus obtained DNA fragments containing the secretion signal sequence can be used as probes for cloning the full length cDNAs.

The proteins encoded by the cDNAs can be distinguished into secretory proteins and membrane proteins by determining the full length nucleotide sequences of the full length cDNAs.

When the obtained clone DNA, whether it is obtained by the random sequence analysis or the signal sequence trap method, codes for a membrane protein, the specific antibody can be obtained by the above method using the synthetic peptide prepared based on the amino acid sequence presumed from the nucleotide sequence as an antigen.

The membrane proteins encoded by the clones include receptors, which may act on the regulation of specific growth of cells having the potential to differentiate into cardiomyocytes or their differentiation into cardiomyocytes. The clone encoding such a receptor can be used in the search for a ligand of the receptor. When the clone codes for a secretion protein, it can be used directly for the growth or differentiation of the cells having the potential to differentiate into cardiomyocytes.

10. Methods for Screening for Growth Factor for Cells having the Potential to Differentiate into Cardiomyocytes and Factor Inducing the Differentiation into Cardiomyocytes

Screening for a growth factor for the cells having the potential to differentiate into cardiomyocytes and a factor inducing their differentiation into cardiomyocytes can be carried out by culturing the cells having the potential to differentiate into cardiomyocytes in a serum-free medium in the presence of a test substance and evaluating the growth or the cardiomyogenic differentiation of the cells.

This screening method is applicable to a wide variety of test substances, for example, secretion proteins such as various cytokines and growth factors, membrane-bound proteins such as cell adhesion molecules, tissue extracts, synthetic peptides, synthetic compounds, and culture broths of microorganisms.

The growth capability can be evaluated by examining the colony forming activity, the BrdU uptake, etc.

The colony forming activity can be examined by scattering the cells having the potential to differentiate into cardiomyocytes of the present invention at a low density.

The BrdU uptake can be examined by immunostaining using an antibody which specifically recognizes BrdU.

The cardiomyogenic differentiation can be evaluated according to a method using spontaneous beating as an indicator, a method using the expression of a reporter gene introduced into the cells as an indicator, and the like.

The method using the expression of a reporter gene introduced into the cells as an indicator is a method in which a vector DNA comprising the promoter of a gene expressed specifically in cardiomyocytes and a reporter gene is introduced into cells having the potential to differentiate into cardiomyocytes and the expression of the reporter gene as an indicator is examined using the cells.

The reporter gene includes genes encoding GFP (gleen fluorescent protein), luciferase or β-galactosidase, and the like.

The promoter of a gene expressed specifically in cardiomyocytes includes cardiac troponin I (cTNI) (J. Biological Chemistry, 273: 25371-25380 (1998)).

11. Methods for Immortalizing Bone Marrow Cells having the Potential to Differentiate into Cardiomyocytes

When the therapeutic agent according to the present invention is administered to cardiac patients, especially aged patients, it is preferred that the proliferative activity of the cells having the potential to differentiate into cardiomyocytes of the present invention should be potentiated without generating cancer.

The proliferative activity of the cells having the potential to differentiate into cardiomyocytes can be increased without cancer generation by expressing telomerase in the cells.

The methods for expressing telomerase in the cells having the potential to differentiate into cardiomyocytes of the present invention include: a method which comprises inserting TERT gene which is the catalytic subunit of telomerase, specifically, the DNA represented by SEQ ID NO:32 into a retrovirus vector and introducing the resulting vector into the cells having the potential to differentiate into cardiomyocytes; a method which comprises administering a factor inducing the expression of the TERT gene inherent in the cells having the potential to differentiate into cardiomyocytes to the cells having the potential to differentiate into cardiomyocytes; and a method which comprises introducing a vector containing DNA encoding a factor inducing the expression of the TERT gene into the cells having the potential to differentiate into cardiomyocytes.

The above-described factors inducing the expression of the TERT gene can be selected by introducing a vector DNA to which a reporter gene such as GFP (green fluorescent protein), luciferase, P-galactosidase or the like has been inserted, into the cells having the potential to differentiate into cardiomyocytes.

12. Method of Separating Cells having the Potential to Differentiate into Cardiomyocytes using Antibody

The method for obtaining cells in which a target surface antigen is expressed from extirpated various in vivo tissues includes a method using a flow cytometer having a sorting function and a method using magnetic beads.

The sorting function of a flow cytometer can be performed by the droplet charge system, the cell capture system, etc. (Perfect Command of Flow Cytometer, p.14-23, Shujunsha, 1999). In using each of these systems, the expression amount of an antigen can be quantitated by converting the fluorescent intensity emitted from an antibody binding to a molecule expressed on the cell surface into an electric signal. When plural fluorescences are used in combination, the cells can be separated using plural surface antigens. Examples of the fluorescence include FITC (fluorescein insothiocyanate), PE (phycoerythrin), APC (Allo-phycocyanin), TR (TexasRed), Cy3, CyChrome, Red613, Red670, PerCP, TRI-Color, QuantumRed, etc. (Perfect Command of Flow Cytometer, p.3-13, Shujunsha, 1999).

The staining method includes a method in which cells are centrifugally separated from extirpated various in vivo tissues such as bone marrow or umbilical blood, and the cells are stained directly with antibodies, and a method in which the cells are once cultured and proliferated in an appropriate medium and then stained with antibodies.

For staining, the target cells are first mixed with a primary antibody, which recognizes a surface antigen, and incubated on ice for 30 minutes to 1 hour. When the primary antibody is labeled with a fluorescence, the cells are washed and then separated with a flow cytometer. When the primary antibody is not labeled with a fluorescence, the cells are washed and then a secondary antibody labeled with a fluorescence having an activity of binding to the primary antibody is mixed with the cells having reacted with the primary antibody and incubated on ice again for 30 minutes to 1 hour. After washing, the cells stained with the primary and secondary antibodies are separated with a flow cytometer.

By the method using magnetic beads, cells expressing specific target surface antigen can be separated in a large amount. Although this method is inferior in the separation purity to the flow cytometer method as described above, repeated purification ensures a sufficiently high cell purity.

After staining the cells with the primary antibody, the residual primary antibody is eliminated. Then the cells are stained with the secondary antibody bonded to the magnetic beads capable of binding to the primary antibody. After washing away the residual secondary antibody, the cells can be separated using a stand provided with a magnet. The materials and apparatus required in these operations are available from Dynal Biotech.

The magnetic bead method is also usable in eliminating unnecessary cells from cell samples. The StemSep method marketed from Stem Cell Technologies Inc. (Vancouver, Canada) can be used to eliminate these unnecessary cells more efficiently.

Examples of the antibodies to be used in the above-described methods include the antibodies acquired in the above 8, antibodies which recognize hematopoietic cell surface antigens, CD34, CD117, CD14, CD45, CD90, Sca-1, Ly6c or Ly6g, antibodies which recognize vascular endothelial cell surface antigens, Flk-1, CD31, CD105 or CD144, an antibody which recognizes a mesenchymal cell surface antigen, CD140, antibodies which recognize integrin surface antigens, CD49b, CD49d, CD29 or CD41, and antibodies which recognize matrix receptors, CD54, CD102, CD106 or CD44. When these antibodies are used in combination, the target cells can be obtained at a higher purity.

Specifically, in order to obtain CD34-negative, CD117-positive, CD144-negative and CD140-positive cells, CD34-positive cells and CD144-positive cells are eliminated from human bone marrow cells by, for example, the above-described immune magnetic bead method and then a CD117-positive and CD140-positive cell fraction is recovered to separate the target cells.

13. Separation of Cardiomyocyte Precursor Cells using Myocardium-Specific Gene Promoter Reporter Vector

In order to efficiently separate cardiomyocytes or cardiomyocyte precursor cells derived from cells having the potential to differentiate into cardiomyocytes, green fluorescent protein (GFP) of luminous Aequorea can be used as a reporter gene for gene transfer.

Specifically, a vector is constructed by ligating the GFP gene to the downstream of a promoter of a gene specifically expressed in myocardium or a gene specifically expressed in the cells having the potential to differentiate into cardiomyocytes obtained in the above 9. Then, the vector is introduced into the cells having the potential to differentiate into cardiomyocytes. The cells introducing the reporter vector are separated depending on, for example, tolerance to antibiotics followed by the induction of cardiomyogenic differentiation. The differentiation-induced cells exhibit the expression of GFP and emit fluorescence. The cardiomyocytes and cardiomyocyte precursor cells emitting the fluorescence can be easily separated using a flow cytometer (Perfect Command of Flow Cytometer, p.44-52, Shujunsha, 1999).

Examples of the promoter of the gene specifically expressed in myocardium include MLC2v and troponin I.

Examples of the vector include the above-described plasmid vectors for animal cells, and adenovirus vectors.

14. Induction of Differentiation of Cells having the Potential to Differentiate into Cardiomyocytes into Various Cells

(1) Induction of Differentiation of Cells having the Potential to Differentiate into Cardiomyocytes into Adipocytes

Examples of the method for inducing the differentiation of the cells having the potential to differentiate into cardiomyocytes into adipocytes include a method wherein an activator of a nuclear receptor, PPARγ, is added to the medium to give a final concentration of 0.4 to 2 μM. The activator of a nuclear receptor, PPARγ, includes compounds having a thiazolidione skeleton such as troglitazone, pioglitazone, rosiglitazone and the like.

The examples also include a method wherein the cells are cultured in a medium to which dexamethasone, methyl-isobutylxanthine, insulin and indomethacin have been added to a culture of cells confluently grown over a culture dish to give final concentrations of 1 μM, 0.5 mM, 0.01 mg/ml and 0.2 mM, respectively.

(2) Induction of Differentiation of Cells having the Potential to Differentiate into Cardiomyocytes into Chondrocytes

Examples of the method for inducing the differentiation of the cells having the potential to differentiate into cardiomyocytes into chondrocytes include a method wherein aggregates obtained by centrifuging 1×10⁵ to 3×10⁵ cells are cultured in a medium containing TGFβ3 in a final concentration of 0.01 μg/ml.

(3) Induction of Differentiation of Cells having the Potential to Differentiate into Cardiomyocytes into Osteoblasts

Examples of the method for inducing the differentiation of the cells having the potential to differentiate into cardiomyocytes into osteoblasts include a method wherein the cells are cultured in a medium containing dexamethasone, ascorbic acid-2-phosphate and β-glycerophosphate in final concentrations of 0.1 μM, 0.05 mM and 10 mM, respectively.

15. Purification of Stem Cell using Hoechst 33342

Hoechst 33342 is a DNA binding reagents which can stain viable cells. Since the majority of bone marrow cells are vigorously divided, they are stained markedly lightly but immature cells are stained darkly. It is known that this phenomenon becomes significant in cells having immature ability to exclude pigment by ABC (ATP binding cassette) transporter (H. Nakauchi, Protein, Nucleic Acid and Enzyme, 45: 13, 2056-2062 (2000)).

Cells which are stained darkly with Hoechst 33342 can be separated from the bone marrow by staining bone marrow cells with Hoechst 33342 and then analyzing them by carrying out double staining of a short wavelength and a long wavelength by applying UV laser using FACS. Immature cells which do not incorporate Hoechst 33342 can be fractionated as side population (Goodell, M. A. et al., J. Exp. Med., 183: 1797-1806 (1996), http://www.bcm.tmc.edu/genetherapy/goodell/new_site/index2.html).

### BRIEF EXPLANATION OF THE DRAWINGS

FIG. 1 shows a result of the antibody reaction of KUM2 cell (A) or BMSC cell (B) using a biotinylated anti-mouse CD105 antibody which was measured by a flow cytometer. The ordinate and abscissa show the number of cells and the fluorescence intensity, respectively. The area painted out with gray is a result of the antibody reaction, and the solid line is a result of a negative control.

FIG. 2 shows a result of the antibody reaction of KUM2 cell (A) or BMSC cell (B) using a biotinylated anti-mouse Flk1 antibody which was measured by a flow cytometer. The ordinate and abscissa show the number of cells and the fluorescence intensity, respectively. The area painted out with gray is a result of the antibody reaction, and the solid line is a result of a negative control.

FIG. 3 shows a result of the antibody reaction of KUM2 cell (A) or BMSC cell (B) using an FITC-labeled anti-mouse CD31 antibody which was measured by a flow cytometer. The ordinate and abscissa show the number of cells and the fluorescence intensity, respectively. The area painted out with gray is a result of the antibody reaction, and the solid line is a result of a negative control.

FIG. 4 shows a result of the antibody reaction of KUM2 cell (A) or BMSC cell (B) using a biotinylated anti-mouse CD144 antibody which was measured by a flow cytometer. The ordinate and abscissa show the number of cells and the fluorescence intensity, respectively. The area painted out with gray is a result of the antibody reaction, and the solid line is a result of a negative control.

FIG. 5 shows a result of the antibody reaction of KUM2 cell (A) or BMSC cell (B) using an FITC-labeled anti-mouse CD34 antibody which was measured by a flow cytometer. The ordinate and abscissa show the number of cells and the fluorescence intensity, respectively. The area painted out with gray is a result of the antibody reaction, and the solid line is a result of a negative control.

FIG. 6 shows a result of the antibody reaction of KUM2 cell (A) or BMSC cell (B) using an FITC-labeled anti-mouse CD117(c-kit) antibody which was measured by a flow cytometer. The ordinate and abscissa show the number of cells and the fluorescence intensity, respectively. The area painted out with gray is a result of the antibody reaction, and the solid line is a result of a negative control.

FIG. 7 shows a result of the antibody reaction of KUM2 cell (A) or BMSC cell (B) using an FITC-labeled anti-mouse CD14 antibody which was measured by a flow cytometer. The ordinate and abscissa show the number of cells and the fluorescence intensity, respectively. The area painted out with gray is a result of the antibody reaction, and the solid line is a result of a negative control.

FIG. 8 shows a result of the antibody reaction of KUM2 cell (A) or BMSC cell (B) using an FITC-labeled anti-mouse CD45 antibody which was measured by a flow cytometer. The ordinate and abscissa show the number of cells and the fluorescence intensity, respectively. The area painted out with gray is a result of the antibody reaction, and the solid line is a result of a negative control.

FIG. 9 shows a result of the antibody reaction of KUM2 cell (A) or BMSC cell (B) using an FITC-labeled anti-mouse CD90 antibody which was measured by a flow cytometer. The ordinate and abscissa show the number of cells and the fluorescence intensity, respectively. The area painted out with gray is a result of the antibody reaction, and the solid line is a result of a negative control.

FIG. 10 shows a result of the antibody reaction of KUM2 cell (A) or BMSC cell (B) using an FITC-labeled anti- mouse Ly6A/E(Sca-1) antibody which was measured by a flow cytometer. The ordinate and abscissa show the number of cells and the fluorescence intensity, respectively. The area painted out with gray is a result of the antibody reaction, and the solid line is a result of a negative control.

FIG. 11 shows a result of the antibody reaction of KUM2 cell (A) or BMSC cell (B) using an FITC-labeled anti-mouse Ly6c antibody which was measured by a flow cytometer. The ordinate and abscissa show the number of cells and the fluorescence intensity, respectively. The area painted out with gray is a result of the antibody reaction, and the solid line is a result of a negative control.

FIG. 12 shows a result of the antibody reaction of KUM2 cell (A) or BMSC cell (B) using an FITC-labeled anti-mouse Ly6g antibody which was measured by a flow cytometer. The ordinate and abscissa show the number of cells and the fluorescence intensity, respectively. The area painted out with gray is a result of the antibody reaction, and the solid line is a result of a negative control.

FIG. 13 shows a result of the antibody reaction of KUM2 cell (A) or BMSC cell (B) using a biotinylated anti-mouse CD140 antibody which was measured by a flow cytometer. The ordinate and abscissa show the number of cells and the fluorescence intensity, respectively. The area painted out with gray is a result of the antibody reaction, and the solid line is a result of a negative control.

FIG. 14 shows a result of the antibody reaction of KUM2 cell (A) or BMSC cell (B) using an FITC-labeled anti-mouse CD49b antibody which was measured by a flow cytometer. The ordinate and abscissa show the number of cells and the fluorescence intensity, respectively. The area painted out with gray is a result of the antibody reaction, and the solid line is a result of a negative control.

FIG. 15 shows a result of the antibody reaction of KUM2 cell (A) or BMSC cell (B) using an FITC-labeled anti-mouse CD49d antibody which was measured by a flow cytometer. The ordinate and abscissa show the number of cells and the fluorescence intensity, respectively. The area painted out with gray is a result of the antibody reaction, and the solid line is a result of a negative control.

FIG. 16 shows a result of the antibody reaction of KUM2 cell (A) or BMSC cell (B) using an FITC-labeled anti-mouse CD29 antibody which was measured by a flow cytometer. The ordinate and abscissa show the number of cells and the fluorescence intensity, respectively. The area painted out with gray is a result of the antibody reaction, and the solid line is a result of a negative control.

FIG. 17 shows a result of the antibody reaction of KUM2 cell (A) or BMSC cell (B) using an FITC-labeled anti-mouse CD54 antibody which was measured by a flow cytometer. The ordinate and abscissa show the number of cells and the fluorescence intensity, respectively. The area painted out with gray is a result of the antibody reaction, and the solid line is a result of a negative control.

FIG. 18 shows a result of the antibody reaction of KUM2 cell (A) or BMSC cell (B) using an FITC-labeled anti-mouse CD102 antibody which was measured by a flow cytometer. The ordinate and abscissa show the number of cells and the fluorescence intensity, respectively. The area painted out with gray is a result of the antibody reaction, and the solid line is a result of a negative control.

FIG. 19 shows a result of the antibody reaction of KUM2 cell (A) or BMSC cell (B) using an FITC-labeled anti-mouse CD106 antibody which was measured by a flow cytometer. The ordinate and abscissa show the number of cells and the fluorescence intensity, respectively. The area painted out with gray is a result of the antibody reaction, and the solid line is a result of a negative control.

FIG. 20 shows a result of the antibody reaction of KUM2 cell (A) or BMSC cell (B) using an FITC-labeled anti-mouse CD44 antibody which was measured by a flow cytometer. The ordinate and abscissa show the number of cells and the fluorescence intensity, respectively. The area painted out with gray is a result of the antibody reaction, and the solid line is a result of a negative control.

The present invention are illustrated below based on the following examples in more detail.

### BEST MODE FOR CARRYING OUT THE INVENTION

### EXAMPLE 1

Isolation and Culture of Bone Marrow Cells having the Potential to Differentiate into Cardiomyocytes from Mouse Bone Marrow:

Ten 5-weeks-old C3H/He mice were anesthetized with ether and sacrificed by cervical dislocation. Each mouse was laid in half-lateral position and sufficiently disinfected with 70% ethanol. The skin around the femur was widely opened and the quadriceps femoris covering the femur was excised with scissors. The femur was put out of the knee joint with scissors and the muscle on the back side of the femur was removed. Then, the femur was put out of the hip joint with scissors and taken out. After the muscle on the femur was removed with scissors to expose the whole femur, the femur was cut at both ends using scissors. A needle (23G, TERUMO) was attached to a 2.5 ml syringe and about 1.5 ml of IMDM containing 20% FCS was put into the syringe. The needle of the syringe was put into the femur from the cut end of the knee joint side and the culture medium was injected into the bone marrow, whereby bone marrow cells were pressed out of the bone into a test tube. The thus obtained cell were cultured in IMDM supplemented with 20% FCS, 100 mg/ml penicillin, 250 ng/ml streptomycin and 85 mg/ml amphotericin at 33° C. using a 5% CO₂-incubator. As a result of a series of passages, the cells were homogenized into mesenchymal cells and hematopoietic cells disappeared.

After culturing for about 4 months under the above conditions, immortalized cells were selected and diluted to establish 192 cell lines respectively derived from single cells (hereinafter referred to as bone marrow-derived first passage immortalized cell lines). To each of these clone-derived cell lines was added 5-aza-C at a final concentration of 3 μM, and the cells were cultured for 24 hours. After culturing for further 2 weeks in IMDM, clones that produced spontaneously beating cells were selected. Among the bone marrow-derived first passage immortalized cell lines (192 cell lines), three cell lines were found to have the potential to differentiate into cardiomyocytes. One of three cell lines is KUM2. Hereinafter, unless otherwise indicated, the bone marrow cell KUM2 and mouse bone marrow-derived pluripotent stem cells (BMSC) described below were cultured in IMDM supplemented with 20% FCS, 100 mg/ml penicillin, 250 ng/ml streptomycin and 85 mg/ml amphotericin at 33° C. using a 5% CO₂-incubator. When the KUM2 cells were exposed to 5-aza-C having a final concentration of 3 μM for 24 hours, nonspecific differentiation into spontaneously beating cardiomyocytes was induced. However, the frequency is very low (one or less per 10⁷ cells).

However, cells surrounding the spontaneously beating cells derived from the KUM2 cells were collected using a cloning syringe to observe at least two cells of the mouse bone marrow-derived pluripotent stem cells (BMSC) having a high proliferation potentiality (FERM BP-7043) and cells differentiated into cardiomyocytes by proliferation under limited times (hereinafter referred to as “cardiomyocyte precursor cells”). BMSC cells isolated by cloning syringe was cloned by selecting immortalized cells in the course of multiple passage. It was observed that the differentiation of the BMSC cells was induced at least 100 times as efficient as the parent cell line, KUM2. And to the cardiomyocyte precursor cells, 5-aza-C was added, followed by culturing for 24 hours, and further culturing in IMDM for 2-3 weeks, so that a larger number of spontaneously beating cells were efficiently obtained. The cardiomyocyte precursor cells showed mononuclear fibroblast-like morphology under the proliferation conditions and expression of myocardial contractile proteins was hardly observed. However, induction of final differentiation with 5-aza-C caused a remarkable change in the morphology of the cells.

About one week after the induction of differentiation, parts of cells showed enlargement of cytoplasm and showed a ball-like or stick-like appearance. Such cells began spontaneously beating afterwards but spontaneous beating was still rare at this stage. Two weeks after the induction of differentiation, the cells began spontaneously beating. The spontaneously beating cells connected lengthwise with one another to form myotube-like structures. Three weeks after the induction of differentiation, many cells were connected in a column and simultaneously contracted. Four weeks after the induction of differentiation, all of the directly connected cells on the culture dish showed simultaneous contraction and formed a myocardial tissue-like structure. The heart of a mouse contracts at a heart rate of 300-400 per minute. On the other hand, the cardiomyocytes differentiated from the cells derived from mouse adult bone marrow showed regular contraction at a rate of 120-250 per minute under the culture conditions.

### EXAMPLE 2

Characteristics of the Cardiomyocytes Derived from Mouse Bone Marrow Cells:

The spontaneously beating cardiomyocyte-like cells produced from the bone marrow cells were examined for the characteristics of cardiomyocytes.

Total RNAs were obtained from the bone marrow-derived first passage immortalized cell line, the mouse bone marrow-derived pluripotent stem cells (BMSC), and the cardiomyocytes derived from the cardiomyocyte precursor cells, which were obtained in Example 1, using Trizol Reagents (manufactured by GIBCO BRL). Then, first strand cDNAs were synthesized from the total RNAs as the substrates using SuperscriptII reverse transcriptase (manufactured by GIBCO BRL).

In order to examine the expression of cardiomyocyte-specific genes, quantitative PCR was carried out using the first strand cDNAs as the substrates and using the synthetic DNAs having the nucleotide sequences represented by SEQ ID NOS:33 to 58. As the cardiomyocyte-specific genes, ANP and BNP, which are natriurectic peptides, α-MHC and β-MHC, which are myosin heavy chains, α-skeletal actin and β-skeletal actin, which are actins, MLC-2a and MLC-2v, which are myosin light chains, and Nkx2.5/Csx, GATA4, TEF-1, MEF-2C, MEF-2D and MEF-2A, which are cardiomyocyte-specific transcription factors, were employed.

For the amplification of the above genes, the synthetic DNAs having the nucleotide sequences shown in the following SEQ ID NOS were respectively used: ANP, SEQ ID NOS:33 and 34; BNP, SEQ ID NOS:35 and 36; a-MHC, SEQ ID NOS:37 and 38; β-MHC, SEQ ID NOS:39 and 40; α-skeletal actin, SEQ ID NOS:41 and 42; β-skeletal actin, SEQ ID NOS:43 and 44; MLC-2a, SEQ ID NOS:45 and 46; MLC-2v, SEQ ID NOS:47 and 48; Nkx2.5/Csx, SEQ ID NOS:49 and 50; GATA4, SEQ ID NOS:51 and 52; TEF-1, SEQ ID NOS:53 and 54; MEF-2C, SEQ ID NOS:55 and 56; MEF-2D, SEQ ID NOS:57 and 58; and MEF-2A, SEQ ID NOS:59 and 60.

In cardiomyocytes produced by induced differentiation in vivo, myocardial contractile proteins have different isoforms according to the difference in stage, i.e., fetal period, new-born period or maturation period, or the difference in type, i.e., atrial or ventricular, so that the rate and energy efficiency of myocardial contraction may vary appropriately.

In the case of the bone marrow cells which differentiate into cardiomyocytes in vitro, a-skeletal actin was expressed at higher levels than a-cardiac actin in the expression pattern of isoforms; β-MHC was expressed at higher levels than α-MHC in the myosin heavy chain; and MLC-2v was expressed, whereas MLC-2a expression was not observed in the myosin light chain.

After the induction of differentiation of the bone marrow cells into cardiomyocytes in vitro, the expression of the natriuretic peptides, ANP and BNP, was observed. In view of the above expression pattern of myocardial contractile proteins, it is considered that the bone marrow cells which differentiated into cardiomyocytes in vitro have a phenotype specific to fetal ventricular cardiomyocytes.

In the bone marrow cells which differentiated into cardiomyocytes in vitro, the expression of genes coding for Nkx2.5/Csx, GATA4, MEF-2A, MEF-2C, MEF-2D or TEF-1 was observed. The genes coding for these transcription factors were not expressed in the bone marrow-derived first passage immortalized cell lines during proliferation. In the bone marrow-derived cardiomyocyte precursor cells during proliferation, the expression of genes coding for Nkx2.5/Csx, GATA4 or MEF-2C was observed. The expression of MEF-2A and MEF-2D was induced later with the induction of cardiomyogenic differentiation.

The action potentials of the bone marrow cells which differentiated into cardiomyocytes in vitro were recorded using glass microelectrodes. The cells were cultured in IMDM supplemented with 1.49 mM CaCl₂, 4.23 mM KCl and 25 mM HEPES (pH 7.4), and the action potentials of the cells were measured at 25° C. under an inverted phase-contrast optic (Diaphoto-300, manufactured by Nikon). The glass microelectrodes were filled with 3M KCl and the electrode resistance was set at 15-30 Ω in the glass microelectrodes. The membrane potentials were measured with current clamp mode using MEZ-8300 (manufactured by Nihon Kohden). The data were recorded on thermal recording papers using RTA-1100M (manufactured by Nihon Kohden). As a result, it was found that the bone marrow cells which differentiated into cardiomyocytes in vitro were classified into two types of action potentials: one is sinus node-like action potential and the other is ventricular myocyte-like action potential. These two type cells of action potentials had the following characteristics in common: (1) a long action potential duration, (2) a relatively shallow resting potential, (3) pacemaker-like slow depolarization of resting potential. The ventricular myocyte-like action potential showed the peak- and dome-like pattern having the phase 1 action potential. The sinus node-like action potential showed the action potential duration, diastolic membrane potential and action potential amplitude which are similar to those previously reported with the action potentials of sinus node cells of rabbits and rats. In comparison, the ventricular myocyte-like action potential had a tendency to show a deep resting membrane potential and a high action potential amplitude. During the 2-3 weeks after the induction of differentiation, the sinus node-like action potential was recorded for all the cells. The ventricular myocyte-like action potential was first recorded about 4 weeks after the induction of differentiation and its incidence gradually increased with the passage of time.

### EXAMPLE 3

Stimulation of Cardiomyogenic Differentiation using Cytokine:

The following experiment was conducted to investigate the stimulating effect of cytokines on the cardiomyogenic differentiation of the mouse bone marrow cells having the potential to differentiate into cardiomyocytes induced by 5-aza-C.

The mouse bone marrow-derived pluripotent stem cells (BMSC) having the potential to differentiate into cardiomyocytes were plated into 60-mm culture dishes and 60 mm fibronectin-coated dishes (Becton Dickinson) at a density of 2×10⁴ cells/ml and cultured at 33° C. in a 5% CO₂-incubator.

On the next day, 5-aza-C was added to each culture medium in a final concentration of 3 μM, followed by culturing with the following 3 different treatments, with addition of PDGF (culture dish A), both PDGF and retinoic acid (culture dish B) or without addition of any compound (culture dish C) (final concentration: PDGF, 10 ng/ml; retinoic acid, 10⁻⁹ M).

On the next day, the medium was replaced with a fresh medium to remove 5-aza-C therefrom. Then, PDGF was added to the culture dish A until the final concentration of PDGF came to be 10 ng/ml, while PDGF and retinoic acid were added to the culture dish B until the final concentrations of PDGF and retinoic acid came to be 10 ng/ml and 10⁻⁹ M, respectively. Two and four days thereafter, the medium was replaced and the PDGF or retinoic acid was further added.

Four weeks after the addition of the chemicals, the cell morphology was observed with a phase-contrast microscope. As a result, about 30% of the cells in the culture dish containing 5-aza-C alone differentiated into myotubes, while about 40% of the cells in the culture dish containing PDGF and about 50% of the cells in the culture dish containing PDGF together with retinoic acid differentiated into myotubes. In the three groups of the fibronectin-coated dishes, the ratio of the cells differentiated into myotubes was about 10% higher than in the three groups of the culture dishes.

RNAs were collected from the myotubes thus obtained. And genes expressed in the myotubes were analyzed with quantitative PCR analysis using the synthetic oligonucleotides represented by SEQ ID NOS:71 to 78. As a result, PDGF or retinoic acid promoted the expression of MyoD and fTnI genes relating to a skeletal muscle but not cTnI or ANP specifically relating to a myocardium. Next, mouse bone marrow-derived pluripotent stem cells (BMSC) having the potential to differentiate into cardiomyocytes were inoculated in a 60-mm culture dish at a density of 2×10⁴ cells/ml and cultured using an incubator at 33° C. under 5% of CO_{2.}

On the next day, 5-aza-C was added to the liquid culture medium to give a final concentration of 3 μM. Furthermore, five treatments differing from each other were performed by adding FGF-8 to give a final concentration of 10 ng/ml (culture dish D); adding ET-1 to give a final concentration of 10 ng/ml (culture dish E); adding a midkine to give a final concentration of 10 ng/ml (Culture dish F); adding BMP4 to give a final concentration of 10 ng/ml (culture dish G); and adding no compound (culture dish H), followed by culturing.

On the next day, the medium was replaced by a fresh medium to eliminate 5-aza-C therefrom. Then, FGF-8 was added to the culture dish D to give a final concentration of 10 ng/ml; ET-1 was added to the culture dish E to give a final concentration of 10 ng/ml; the midkine was added to the culture dish F to give a final concentration of 10 ng/ml; and BMP4 was added to the culture dish G to give a final concentration of 10 ng/ml, followed by culturing. Two and four days thereafter, the medium was replaced and the FGF-8, ET-1, midkine or BMP4 was further added.

Four weeks after the addition of 5-aza-C, the cell morphology was observed with a phase-contrast microscope. As a result, about 30% of the cells in the culture dish containing 5-aza-C alone differentiated into myotubes, while about 50% of the cells in the culture dishes containing FGF-8, ET-1, midkine or BMP4 differentiated into myotubes respectively.

RNAs were collected from the myotubes thus obtained. And genes expressed in the myotubes were analyzed with quantitative PCR using the synthetic oligonucleotides represented by SEQ ID NOS:71 to 78. As a result, the FGF-8, ET-1, midkine and BMP4 each individually promoted the expression of cTnI and ANP gene which are myocardium-specific genes.

### EXAMPLE 4

Induction of Differentiation of Bone Marrow-Derived Stem Cells into Cardiomyocytes using DMSO:

According to the method described in Example 1, mouse bone marrow-derived pluripotent stem cells (BMSC) having the potential to differentiate into cardiomyocytes were obtained and cultured for 24 hours in the presence of 10 μM DMSO instead of 3 μM 5-aza-C. The medium was replaced with IMDM, followed by culturing for 6 weeks.

As a result, the stem cells were induced to differentiate into beating cardiomyocytes. The produced cells expressed Nkx2.5/Csx and GATA4 genes and were found to be cardiomyocytes having the same properties as those obtained by the 5-aza-C treatment. This result indicates that cardiomyogenic differentiation requires demethylation of chromosomal DNA, which is a function common to 5-aza-C and DMSO.

### EXAMPLE 5

Demonstration that Mouse Bone Marrow-Derived Pluripotent Cells having the Potential to Differentiate into Cardiomyocytes are Pluripotent Stem Cells and Cardiomyocyte Precursor Cells:

It was demonstrated above that the beating cells differentiated from the mouse bone marrow-derived pluripotent stem cell (BMSC) have the properties of cardiomyocytes. In this example, a single cell marking experiment was carried out to examine whether cardiomyocyte precursor cells are present in the mouse bone marrow-derived pluripotent stem cells (BMSC) having the potential to differentiate into cardiomyocytes, or whether more undifferentiated stem cells which can differentiate into not only cardiomyocytes, but also, for example, adipocytes and other cell types are present.

Specifically, a GFP gene was inserted into a virus vector and the vector was transfected into a cell for labeling prior to induction of differentiation, and the labeled cell was induced to differentiate to observe what kind of cell is produced by differentiation.

First, retrovirus vector plasmid GAR3-GFP which expresses the GFP gene products and plasmid vector pCMV-Eco which expresses the Ecotropic gene products were treated according to the alkali neutralization method and the PEG precipitation method described in Molecular Cloning, A Laboratory Manual, 2nd ed. to obtain DNAs of high purity.

One day before DNA transfection, 293 cells carrying the gag and pol genes which had reached confluence were passaged into a 10-cm dish by ⅕ dilution and cultured overnight at 37° C. in a 5% CO₂-incubator.

Transfection was carried out as follows.

GAR3-GFP retrovirus vector plasmid DNA (15 μg) and pCMV-Eco plasmid vector DNA (5 μg) were dissolved in 0.5 ml of 250 mM CaCl₂ (pH 6.95). The resulting solution was added dropwise to a 15 ml tube containing 0.5 ml of 2× BBS (50 mM BES (N,N-bis(2-hydroxyethl)-2-aminoethanesulfonic acid), 280 mM NaCl and 1.5 mM Na₂HPO₄ (pH 6.95)) and the tube was allowed to stand at room temperature for 10 minutes. The resulting DNA solution was added dropwise to the 293 cell culture prepared on the preceding day, followed by culturing at 37° C. in a 5% CO₂-incubator. On the next day, the medium was replaced with a fresh medium, followed by culturing at 37° C. in the 5% CO₂-incubator.

Two days after the medium replacement, the culture supernatant was filtered through a 0.45 μm filter (manufactured by Millipore) to recover a solution containing the virus vector. The obtained solution was diluted to 10⁻¹, 10⁻², 10⁻³, 10⁻⁴ and 10⁵ with IMDM.

The mouse bone marrow-derived pluripotent stem cells having the potential to differentiate into cardiomyocytes into which the virus vector was to be introduced were plated into 6-well dishes at a density of 2×10⁴ cells/well on the day before virus infection.

To the diluted virus vector solution, hexadimethine bromide (polybrene) (manufactured by Sigma) was added to give a final concentration of 8 μg/ml. After 2 ml of the culture supernatant of the mouse bone marrow-derived pluripotent stem cells (BMSC) having the potential to differentiate into cardiomyocytes was replaced with 2 ml of the virus solution, culturing was carried out at 33° C. in a 5% CO₂-incubator. Five hours later, the culture supernatant was replaced with a fresh IMDM, followed by culturing at 33° C. in the 5% CO₂-incubator.

After culturing for 2 days, the cells were observed for GFP expression by a fluorescence microscope to obtain cell populations containing one GFP-positive cell in 1000 cells.

The obtained cells were plated into 35 mm glass base dishes (manufactured by Asahi Techno Glass) at a density of 8×10³ cells/dish followed by culturing at 33° C. in a 5% CO₂-incubator.

On the next day, 5-aza-C (manufactured by Sigma), PDGF-BB (manufactured by Peprotech) and all trans retinoic acid (manufactured by Sigma) were added to the dishes to give final concentrations of 3 μM, 10 ng/ml and 10⁻⁹ M, respectively. Two days and four days after the addition, the medium was replaced with a fresh medium and PDGF-BB (hereinafter referred to as “PDGF”) and all trans retinoic acid were added at the same concentrations as above.

Four weeks after, the cultures were observed under a fluorescence microscope to examine the mode of differentiation of the GFP-positive cells. As a result, the following three kinds of cell populations were observed; cell populations in which all the GFP-positive cells were cardiomyocytes; cell populations in which cardiomyocytes and undifferentiated stem cells were GFP-positive; and cell populations in which cardiomyocytes, adipocytes and undifferentiated stem cells were GFP-positive. It has thus been found that differentiation is stochastically derived from pluripotent stem cells through myocardial stem cells and then cardiomyocyte precursor cells. This result also indicates that the mouse bone marrow cells having the potential to differentiate into cardiomyocytes comprise pluripotent stem cells.

### EXAMPLE 6

Promotion of Differentiation into Cardiomyocytes by Forced Expression of Transcription Factors:

The following experiment was carried out to examine the effect of the forced expression of transcription factors relating to cardiomyogenic differentiation on the cardiomyogenic differentiation of the bone marrow-derived pluripotent stem cells (BMSC) having the potential to differentiate into mouse cardiomyocytes.

That is, the Nkx2.5/Csx or GATA4 gene was introduced into the cells using a virus vector prior to induction of differentiation, and then the cells were induced to differentiate to examine the efficiency of cardiomyogenic differentiation.

In order to express the Nkx2.5/Csx, Nkx2.5/Csx was inserted into retrovirus vector plasmid PCLNCX (manufactured by Imgenex) to prepare pCLNC-Nkx2.5/Csx.

Furthermore, in order to express GATA4, GATA4 was inserted into plasmid pCLPCX in which the G418-resistant gene portion in retrovirus vector plasmid pCLNCX (manufactured by Imgenex) had been replaced with puromycin-resistant genes, to prepare pCLPC-GATA4. The retrovirus vector plasmids pCLNC-Nkx2.5/Csx and pCLPC-GATA4 and plasmid vector pCMV-Eco (manufactured by Imgenex) which expresses the Ecotropic gene were treated according to the alkali neutralization method and the PEG precipitation method described in Molecular Cloning, A Laboratory Manual, 2nd ed., etc. to obtain DNAs having high purity.

One day before DNA transfection, 293 cells carrying the gag and pol gene which had reached confluence were passaged into a 10-cm dish by ⅕ dilution followed by culturing overnight at 37° C. in a 5% CO₂-incubator.

Transfection was carried out as described below.

15 μg of retrovirus vector DNA, pCLNC-Nkx2.5/Csx or pCLPC-GATA4, and 5 μg of plasmid vector, pCMV-Eco, were added and dissolved in 0.5 ml of 250 mM CaCl₂ (pH 6.95). The resulting solution was added dropwise to a 15 ml tube containing 0.5 ml of 2×BBS (50 mM BES (N,N-bis(2-hydroxyethl)-2-aminoethanesulfonic acid), 280 mM NaCl and 1.5 mM Na₂HPO₄ (pH 6.95)) and the tube was allowed to stand at room temperature for 10 minutes. The resulting DNA solution was added dropwise to the 293 cell culture prepared on the preceding day, followed by culturing at 37° C. in a 5% CO₂-incubator. On the next day, the medium was replaced with a fresh medium, followed by culturing at 37° C. in the 5% CO₂-incubator.

Two days after the medium replacement, the culture supernatant was filtered through a 0.45 μm filter (manufactured by Millipore) to recover a solution containing the virus vector.

The mouse bone marrow-derived pluripotent stem cells (BMSC) having the potential to differentiate into cardiomyocytes into which the virus vector was to be introduced were plated into 6-well dishes at a density of 2×10⁴ cells/well on the day before virus infection.

To the obtained virus vector solution, hexadimethrine bromide (polybrene) (manufactured by Sigma) was added to give a final concentration of 8 μg/ml. The culture medium was replaced with the culture medium for the mouse bone marrow-derived pluripotent stem cells (BMSC) having the potential to differentiate into cardiomyocytes, followed by culturing at 33° C. in a 5% CO₂-incubator. Five hours later, the medium was replaced with a fresh IMDM, followed by culturing at 33° C. in the 5% CO₂-incubator, and further culturing for 2 days.

G418 was added to the cells infected with the virus produced by transferring pCLNC-Nkx2.5 and pCMV-Eco to give a final concentration of 300 μg/ml, followed by culturing for further 7 days.

Separately, puromycin was added to the cells infected with the virus produced by transferring PCLPC-GATA4 and pCMV-Eco to give a final concentration of 300 ng/ml, followed by culturing for further 7 days.

During this period, both cells partly died and were detached from the dish. The surviving cells were suspended with trypsin followed by plating into new culture dishes.

The obtained stable transformants for expression of Nkx2.5/Csx or GATA4 were induced for differentiation by the method in the above Example 3, and thus the differentiation efficiency into cardiomyocytes was examined.

The KNX2.5 forced expressing bone marrow cells (BMSC-KNx2.5) having the potential to differentiate into cardiomyocytes and the GATA4 forced expressing bone marrow cells (BMSC-GATA4) having the potential to differentiate into cardiomyocytes were plated into 60-mm culture dishes at a density of 2×10⁴ cells/ml, followed by culturing at 33° C. in a 5% CO₂-incubator. On the next day, 5-aza-C was added to each culture medium to give a final concentration of 3 μM. After continuing the culturing at 33° C. in a 5% CO₂-incubator for further 24 hours, the medium was replaced with a fresh medium to eliminate 5-aza-C, followed by culturing for additional 4 weeks. When observed with a phase-contrast microscope, the number of myotube showed no large change caused by the forced expression of Nkx2.5/Csx or GATA4. Next, RNAs were collected from the myotubes thus obtained and genes expressed in the myotubes were analyzed with quantitative PCR using the synthetic oligonucleotides represented by SEQ ID NOS:71 to 78. As a result, it was observed that the forced expression of Nkx2.5/Csx or GATA4 promoted the expression of cTnI and ANP which are myocardium-specific genes.

To simultaneously express both of the Nkx2.5/Csx and GATA4 genes in bone marrow cells having the potential to differentiate into cardiomyocytes, a retrovirus vector plasmid pCLPC-GATA4 was treated as described above and bone marrow cells (BMSC-KNX2.5) with the forced expression of Nkx2.5/Csx having the potential to differentiate into cardiomyocytes were infected with the recombinant virus thus constructed. Next, puromycin was added to give a final concentration of 300 ng/ml to obtain a drug-tolerant clone (BMSC-Nkx2.5-GATA4).

The Nkx2.5/Csx and GATA4 co-forced expressing bone marrow cells (BMSC-Nkx2.5-GATA4) having the potential to differentiate into cardiomyocytes were plated into a 60-mm culture dish at a density of 2×10⁴ cells/ml, followed by culturing at 33° C. in a 5% CO₂-incubator.

On the next day, 5-aza-C was added to the culture medium to give a final concentration of 3 μM. After culturing at 33° C. in a 5% CO₂-incubator for further 24 hours, the medium was replaced with a fresh medium to eliminate 5-aza-C, followed by culturing for 4 weeks. When observed with a phase-contrast microscope, the number f myotube showed no large change caused by the forced expression of the Nkx2.5/Csx and GATA4 genes. However, the number of beating cardiomyocyte was 50 times or more elevated than bone marrow cells with no forced expression of these genes having the potential to differentiate into cardiomyocytes. Next, RNAs were collected from the myotubes thus obtained and genes expressed in the myotubes were analyzed with quantitative PCR using the synthetic oligonucleotides represented by SEQ ID NOS:71 to 78. As a result, it was observed that the forced expression of Nkx2.5/Csx and GATA4 promoted the expression of cTnI and ANP which are myocardium-specific genes.

### EXAMPLE 7

Promotion of Differentiation into Cardiomyocytes by Combination of the Forced Expression of Transcriptional Factors with Cytokines:

By combining the above-described transcriptional factors (Nkx2.5/Csx and GATA4) promoting the differentiation into cardiomyocytes with cytokines (FGF-8, ET-1, midkine and BMP4), effects on the differentiation into cardiomyocytes were analyzed.

The Nkx2.5/Csx and GATA4 co-forced expressing bone marrow cells (BMSC-Nkx2.5-GATA4) having the potential to differentiate into cardiomyocytes were plated into a 60-mm culture dish at a density of 2×10⁴ cells/ml and cultured at 33° C. in a 5% CO₂-incubator.

On the next day, 5-aza-C was added to the culture medium to give a final concentration of 3 μM. Furthermore, 5 treatments differing from each other were carried out by adding FGF-8 to give a final concentration of 10 ng/ml (culture dish I); adding ET-1 to give a final concentration of 10 ng/ml (culture dish J); adding midkine to give a final concentration of 10 ng/ml (culture dish K); adding BMP4 to give a final concentration of 10 ng/ml (culture dish L); and adding nothing (culture dish M), followed by culturing.

On the next day, the medium was replaced with a fresh medium to eliminate 5-aza-C. Then, FGF-8 was added to the culture dish I to give a final concentration of 10 ng/ml; ET-1 was added to the culture dish J to give a final concentration of 10 ng/ml; midkine was added to the culture dish K to give a final concentration of 10 ng/ml; and BMP4 was added to the culture dish L to give a final concentration of 10 ng/ml, followed by culturing. Two and four days thereafter, furthermore, the medium was replaced, and the FGF-8, ET-1, midkine or BMP4 was added.

Four weeks after the addition of 5-aza-C, the cell morphology was observed with a phase-contrast microscope. As a result, about 30% of the cells in the culture dish containing 5-aza-C alone were converted into myotubes, while about 50% of the cells in the culture dishes containing FGF-8, ET-1, midkine or BMP4 differentiated into myotubes respectively. On the other hand, the addition of FGF-8, ET-1, midkine or BMP4 caused no increase in beating cardiomyocytes.

From the myotubes thus obtained, RNAs were collected and genes expressed in the myotubes were subjected to quantitative PCR analysis using the synthetic oligonucleotides represented by SEQ ID NOS:71 to 78. As a result, the FGF-8, ET-1, midkine and BMP4 did not further promote the expression of cTnI and ANP which had been promoted by the forced expression of Nkx2.5/Csx and GATA4.

### EXAMPLE 8

Transplantation of Mouse Having the Potential to Differentiate into Cardiomyocytes into Heart:

In order to examine whether or not bone marrow cells having the potential to differentiate into cardiomyocytes would differentiate into myocardia and thus take into the heart, the GFP labeled bone marrow cells (BMSC-GFP) having the potential to differentiate into cardiomyocytes as prepared in Example 5 were employed as donor cells for the transplantation into mouse. Specifically, the following procedure was performed. The GFP-labeled BMSCs were transiently treated with 5-aza-C for 24 hours, then suspended in PBS to give a concentration of 1×10⁸ cells/ml and stored on ice until immediately before the transplantation. It had been confirmed by 0.05% erythrosine-staining that BMSCs could survive at a ratio of about 95%.

On the other hand, the recipient C3H/He mice (available from Charles River Japan) were anesthetized with ether, and the anesthesia was maintained by intraperitoneally administering 30 mg of thiopental using a Terumo syringe (1 ml) manufactured by Terumo Corp. The legs of each mouse were fixed on a cork board with tape, and its upper jaw was also fixed on the cork board with rubber in such a manner that the neck leaned back. At this stage, electrocardiography electrodes were put into both upper limbs and right side lower limb to monitor the electrocardiogram. Next, the cervix was incised about 1 cm along the trachea using Mayo scissors (NONAKA RIKAKI CO., LTD, NK-174-14), the thyroid gland was stripped to the right and left sides using a baby cotton swab manufactured by Hakujuji, and then muscles around the trachea were incised using micro scissors (NONAKA RIKAKI CO., LTD, NY-334-08) to expose the trachea. Next, the trachea was incised in about 1 mm width using a micro-feather (a surgical knife), a needle of Surflow Flash (22G) manufactured by Terumo deformed into J-shape was inserted into the opening and taken out from the oral cavity, and then the syringe of Surflow Flash (20G) was inserted into the trachea using the needle as a guide. By connecting a respirator (MODEL SN-480-7, manufactured by SHINANO SEISAKUSHO) to the syringe, 100% oxygen was flowed at a rate of 1 ml/minute to start artificial respiration with a tidal volume of 1 ml and a respiration frequency of 120/minute. Since air leaks out from the guide needle-inserted opening, the skin around the trachea was closed by covering the trachea using mosquito forceps (manufactured by NONAKA RIKAKI CO., LTD.). Next, a region of about 2 cm from the sternal pedicel toward the cervix was incised using Mayo scissors and then the sternum was incised about 2 cm from the sternal pedicel toward the cervix. Bleeding was stopped using a bipolar electric knife, and then a 30G needle (metal hub exchange needle N730) manufactured by GL Science was connected to the Terumo syringe (1 ml) manufactured by Terumo Corp and 0.1 ml of a solution prepared by suspending the donor cells in PBS was injected into the apical region. Next, the sternum and the skin were closed using 4-0 ETHIBOND X761 manufactured by ETHICON, and the skin of the cervix was closed using the same suture. After confirmation of the turn up of spontaneous respiration, the respirator was taken out, and an infant warmer was heated to 37° C. to wait vigilance of the animal therein. Also, the procedure of this test was carried out using DESIGN FOR VISON 4.5× SURGICAL TELESCOPES.

Tissues were taken out from the mouse 77 days after the transplantation, fixed with 10% formalin and embedded in paraffin. The embedded tissues were sliced with a microtome into pieces of 6 μm in thickness and adhered to slide glasses which had been coated with poly-L-lysine. After eliminating paraffin by immersing in 10% xylene, the samples were washed with ethanol and then immersed in 0.3% H₂O₂ for 30 minutes, followed by a pretreatment for the antibody reaction.

Then, the samples were washed with PBS and blocked by reacting with a 5% normal swine serum solution. After blocking, the samples were washed with PBS and then subjected to the antibody reaction by allowing to stand at 4° C. overnight together with a mouse anti-GFP monoclonal antibody (manufactured by CLONTECH). After washing with PBS, the samples were allowed to react with a peroxidase-labeled dextran-bonded goat anti-mouse immunoglobulin antibody (manufactured by DACO) at room temperature for 30 minutes. After washing with PBS again, a coloring solution (10 μg/ml 3,3′-diaminobenzidine (DAB) tetrahydrochloride, 0.01% H₂0₂, 0.05 M Tris-HCl (pH 6.7)) was added and allowed to react for about 10 minutes. Then, the reaction mixture was washed with PBS to stop the reaction. Furthermore, the slide glasses were stained with methyl green. The part of continuous pieces were stained with hematoxylin/eosin to clarify the morphology of the tissue pieces.

As a result, GFP-positive cells were observed in the cardiomyocytes and the vascular endothelial cells.

Thus, it can be concluded that the transplanted mouse bone marrow cells had differentiated into the cardiomyocytes and the vascular endothelial cells.

### EXAMPLE 9

Promotion of Differentiation into Cardiomyocytes by Cultured Cardiomyocyte-Derived Factor:

As shown in Example 8, the bone marrow cells (BMSC) having the potential to differentiate into cardiomyocytes were differentiated into the cardiomyocytes when transplanted into the heart. This result suggests that cardiomyocytes per se expresses a factor inducing the differentiation of bone marrow cells into cardiomyocytes. To examine this hypothesis, a mouse fetal heart was taken out from a C3H/He mouse on the day 16 of pregnancy and a primary culture cell line of cardiomyocytes (hereinafter referred to as the “cultured cardiomyocytes”) was established in accordance with a publicly known method (Development of Method for Studying Heart and Blood, ed. by Setsuro Ebashi, Gakkai Shuppan Senta, (1983)).

To examine whether or not a factor secreted from the cultured cardiomyocytes has an activity of promoting heart differentiation, 5×10⁶ cultured cardiomyocytes were cultured in a culture dish for 72 hours. Next, the culture supernatant was filtered through a 0.45 μm filter (manufactured by Millipore). The culture supernatant thus filtered was mixed with the equivalent amount of a medium to give a culture medium (hereinafter referred to as the “conditioned medium”) containing the factor secreted from the cultured cardiomyocytes.

Bone marrow cells (BMSC) having the potential to differentiate into cardiomyocytes or Nkx2.5 and GATA4 forced expressing bone marrow cells (BMSC-Nkx2.5-GATA4) having the potential to differentiation into cardiomyocytes were cultured 6-cm culture dishes at a density of 1×10⁵ cells and then the medium was replaced with the conditioned medium. At this point, 5-aza-C was added to give a final concentration of 3 μM. On the next day, the medium was replaced with the fresh conditioned medium, followed by culturing for further 4 weeks. During this period, the medium was replaced with the fresh conditioned medium once 3 days. Thus, it was observed that myotubes derived from the bone marrow cell (BMSC) having the potential to differentiate into cardiomyocytes showed no increase but the expression of the two myocardium-specific genes (ANP and cTnI) was promoted by the addition of the conditioned medium. In case of the Nkx2.5 and GATA forced expressing bone marrow cells (BMSC-Nkx2.5-GATA4) having the potential to differentiate into cardiomyocytes, on the other hand, the myotubes showed no increase and the expression of the two myocardium-specific genes (ANP and cTnI) was promoted at the same level as in Nkx2.5 and GATA4 by the addition of the conditioned medium, showing no promoting effect.

Next, it was examined whether or not cardiomyocyte-expressing extracellular matrix (ECM) has an activity of promoting the differentiation into cardiomyocytes, culture dishes wherein cardiomyocytes had been cultured were treated with 0.45% trypsin/EDTA for about 30 minutes to eliminate the cardiomyocytes. Thus, culture dishes coated with the extracellular matrix of the cultured cardiomyocytes (hereinafter referred to as the “ECM-coated dishes”) were prepared. Subsequently, bone marrow cells (BMSC) having the potential to differentiate into cardiomyocytes or compulsively both Nkx2.5 and GATA 4 genes-expressed bone marrow cells (BMSC-Nkx2.5-GATA4) having the potential to differentiate into cardiomyocytes were cultured in these 6-cm culture dishes at a density of 1×10⁵ cells and then 5-aza-C was added to give a final concentration of 3 μM. On the next day, the medium was replaced with a fresh medium to eliminate 5-aza-C and the culture was continued for further 4 weeks. During this period, the medium was replaced with a fresh medium once 3 days. Thus, it was observed that myotubes derived from the bone marrow cells (BMSC) having the potential to differentiate into cardiomyocytes showed no increase but the expression of the two myocardium-specific genes (ANP and cTnI) was promoted by the coated dish. In case of the compulsively both Nkx2.5 and GATA4 genes-expressed bone marrow cells (BMSC-Nkx2.5-GATA4) having the potential to differentiate into cardiomyocytes, on the other hand, the myotubes showed no increase and the expression of the two myocardium-specific genes (ANP and cTnI) was promoted at the same level as in Nkx2.5 and GATA4 by the addition of the conditioned medium, showing no promoting effect.

Next, 2×10⁴ cultured cardiomyocytes were co-cultured together with 8×10⁴ bone marrow cells (BMSC) having the potential to differentiate into cardiomyocytes or 8×10⁴ compulsively both Nkx2.5 and GATA4 genes-expressed bone marrow cells (BMSC-Nkx2.5-GATA4) having the potential to differentiate into cardiomyocytes in 6-cm culture dishes. To distinguish the cultured cardiomyocytes from the bone marrow cells, the two types of bone marrow cells (BMSC and BMSC-Nkx2.5-GATA4) were labeled with GFP as in Example 5. On the next day after the co-culturing, 5-aza-C was added to give a final concentration of 3 μM. On the next day, the medium was replaced with a fresh medium to eliminate 5-aza-C, followed by culturing for further 4 weeks. During this period, the medium was replaced with a fresh medium once 3 day. As a result, beating cardiomyocytes were increased about 10 times or more than the case wherein BMSC or BMSC-Nkx2.5-GATA4 were cultured alone. Thus, it was found that the efficiency of the differentiation into cardiomyocytes can be elevated 500 times or more by combining the forced expression of the Nkx2.5 and GATA4 genes with the co-culturing with cardiomyocytes.

### EXAMPLE 10

Analysis of Surface Antigens of KUM2 Cells and BMSCs:

Surface antigens of KUM2 cells and BMSCs were analyzed to clearly differentiate KUM2 cells from BMSCs and develop a method for efficiently isolating and purifying cells having the potentiality of forming myocardium from bone marrow.

The surface antigens employed in the analysis included 20 antigens, i.e., CD105, Flk-1, CD31 and CD144 known as surface antigens of vascular endothelial cells, CD34, CD117(c-kit), CD14, CD45, CD90, Ly6A/E(Sca-1), Ly6c and ly6g known as surface antigens in hematopoietic cells, CD140 known as surface antigens of mesenchymal cells, integrins CD49b, CD49d and CD29 and matrix receptors CD54, CD102, CD106 and CD44.

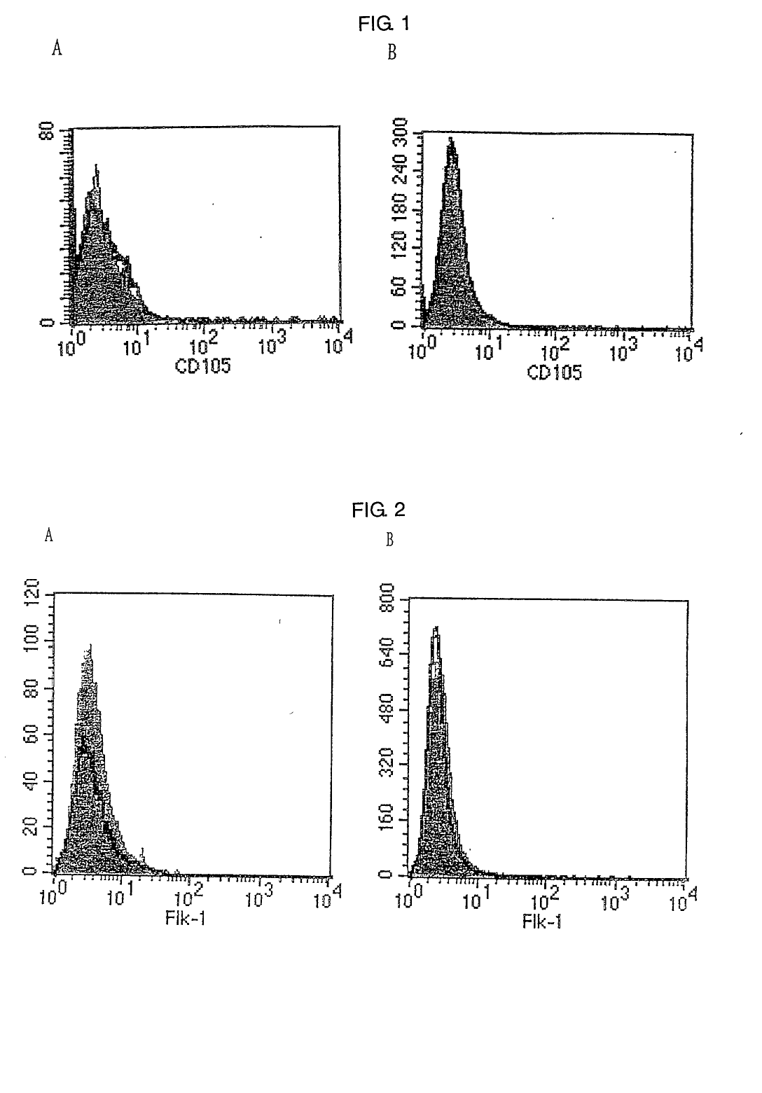

First, 1×10⁴ KUM2 cells or 1×10⁴ BMSC cells were pipetted into a 96-well U-shaped plate. An anti-mouse CD105 antibody (manufactured by Pharmingen), which had been biotin-labeled by a publicly known method (Enzyme Antibody Technique, Gakusai Kikaku (1985)), was added to a buffer for FACS (1% BSA-PBS, 0.02% EDTA, 0.05% NaN₃, pH 7.4), then added to the wells and allowed to react on ice for 30 minutes. As a negative control, rat IgG2a, K-purified antibody (manufactured by Pharmingen) was used. After washing with the buffer twice, 20 μl of streptoavidin-PE (manufactured by Nippon Becton Dickinson) was added. Then the mixture was allowed to react in the dark on ice for 30 minutes, washed with the buffer thrice and suspended in 500 μl of the buffer. The fluorescence intensity was measured with a flow cytometer and it was examined whether or not the fluorescence intensity was increased by adding the antibody. The results are shown in FIG. 1. As a result, it was found that the KUM2 cells and the BMSC cells were both CD105-negative.

Regarding the occurrence of the expression of the Flk-1 antigen, an antibody reaction was carried out in the manner similar to that described above, using a biotinylated anti-mouse Flk-1 antibody (PM-28181D, manufactured by Pharmingen), followed by measurement with a flow cytometer. The results are shown in FIG. 2. As a result, it was found that the KUM2 cells and the BMSC cells were both Flk-1-negative.

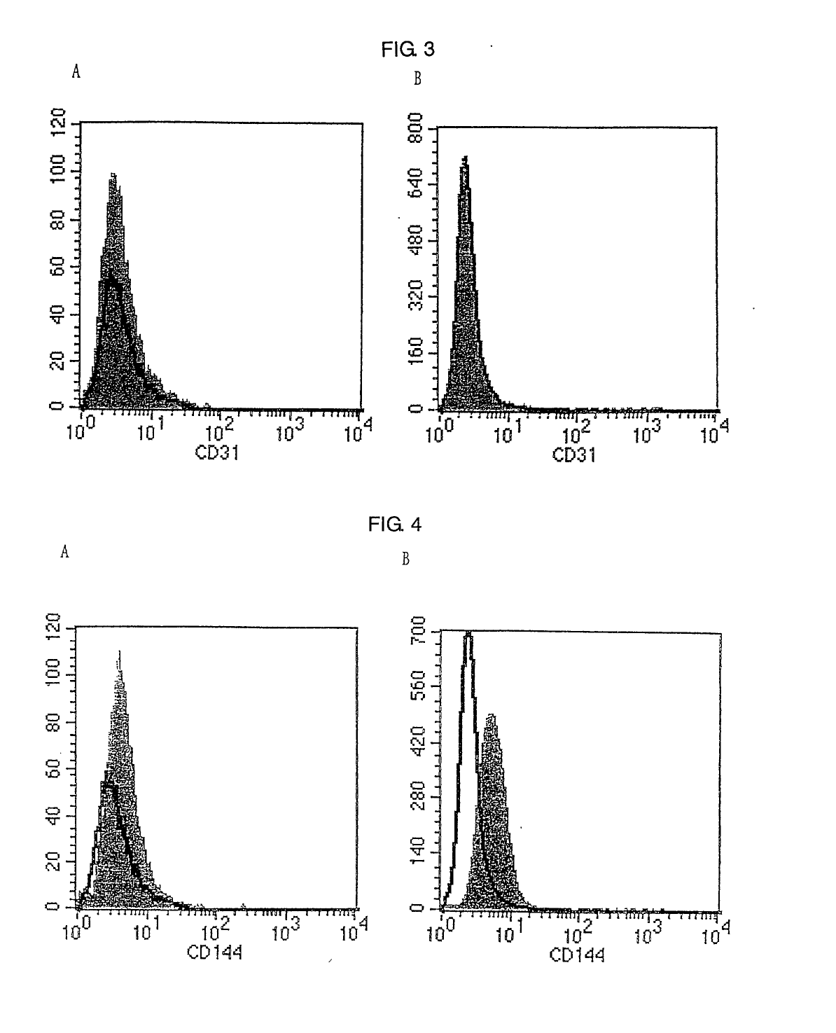

Regarding the occurrence of the expression of the CD31 antigen, an antibody reaction was carried out using an FITC-labeled anti-mouse CD31 antibody (PM-01954D, manufactured by Pharmingen), followed by measurement with a flow cytometer. The results are shown in FIG. 3. As a result, it was found that the KUM2 cells and the BMSC cells were both CD31-negative.

Regarding the occurrence of the expression of the CD144 antigen, an antibody reaction was carried out using a biotinylated anti-mouse CD144 antibody (PM-28091D, manufactured by Pharmingen) followed by measurement with a flow cytometer. The results are shown in FIG. 4. As a result, it was found that the KUM2 cells were CD144-negative, while the BMSC cells were CD144-weak positive.

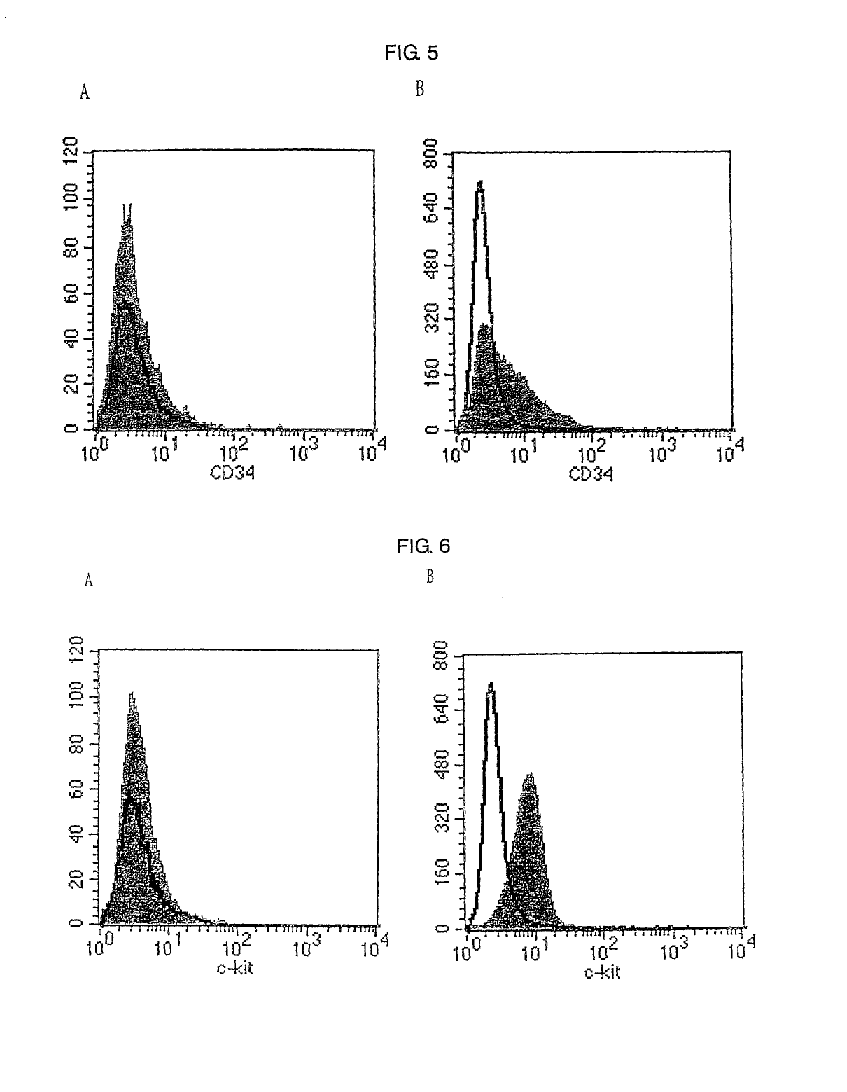

Regarding the occurrence of the expression of the CD34 antigen, an antibody reaction was carried out using an FITC-labeled anti-mouse CD34 antibody (PM-09434D, manufactured by Pharmingen), followed by measurement with a flow cytometer. The results are shown in FIG. 5. As a result, it was found that the KUM2 cells were CD34-negative, while the BMSC cells were a mixture of CD34-positive cells and CD34-negative cells.

Regarding the occurrence of the expression of the CD117(c-kit) antigen, an antibody reaction was carried out using an FITC-labeled anti-mouse CD117 antibody (PM-01904D, manufactured by Pharmingen), followed by measurement with a flow cytometer. The results are shown in FIG. 6. As a result, it was found that the KUM2 cells were CD117-negative, while the BMSC cells were CD117-positive.

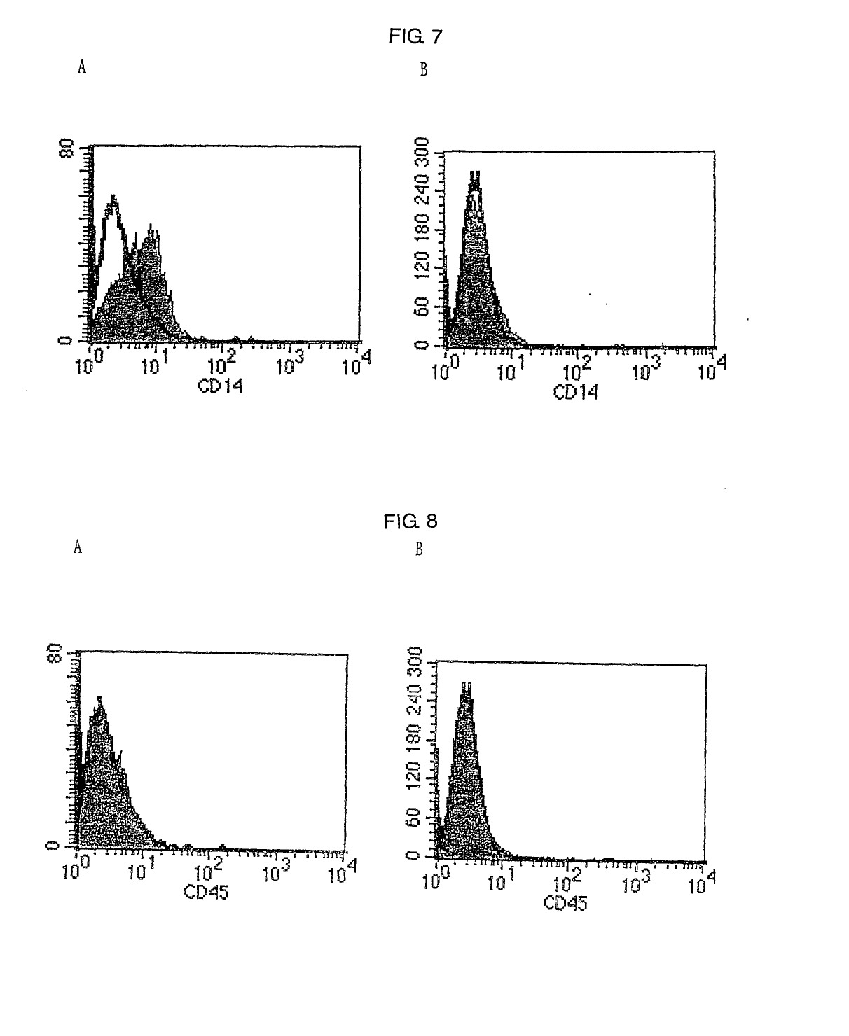

Regarding the occurrence of the expression of the CD14 antigen, an antibody reaction was carried out using an FITC-labeled anti-mouse CD14 antibody (PM-09474, manufactured by Pharmingen), followed by measurement with a flow cytometer. The results are shown in FIG. 7. As a result, it was found that the KUM2 cells were CD14-positive, while the BMSC cells were CD14-negative.

Regarding the occurrence of the expression of the CD45 antigen, an antibody reaction was carried out using an FITC-labeled anti-mouse CD45 antibody (PM-01114, manufactured by Pharmingen), followed by the measurement with a flow cytometer. The results are shown in FIG. 8. As a result, it was found that the KUM2 cells and the BMSC cells were both CD45-negative.

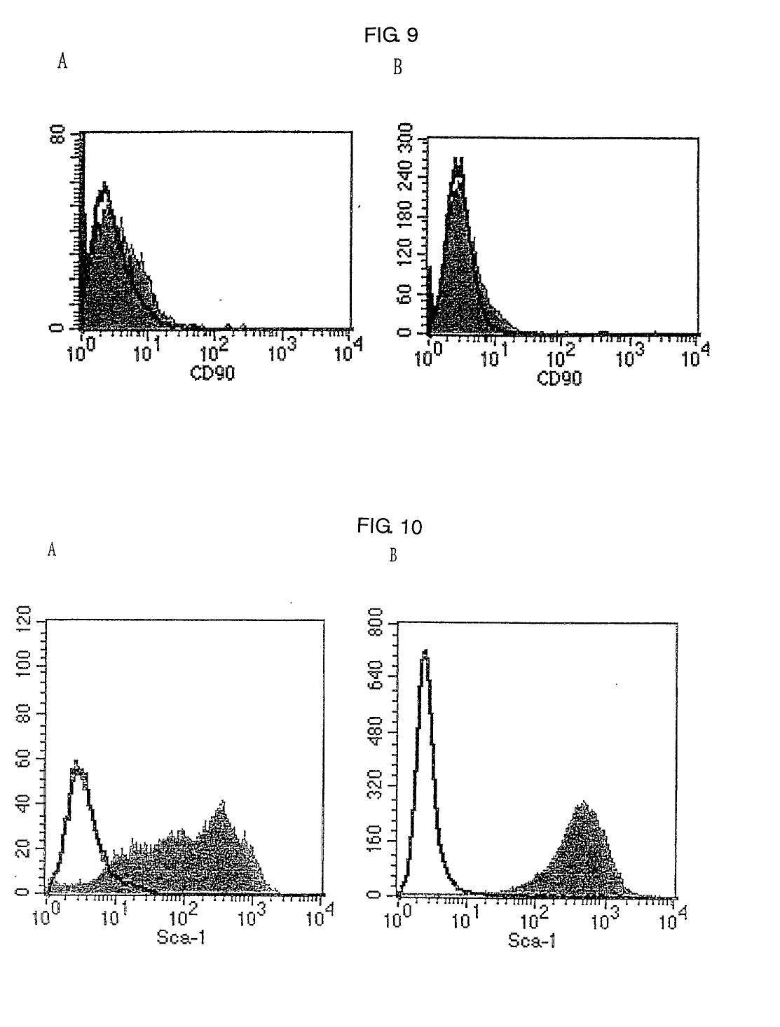

Regarding the occurrence of the expression of the CD90 antigen, an antibody reaction was carried out using an FITC-labeled anti-mouse CD90 antibody (PM-22214, manufactured by Pharmingen), followed by measurement with a flow cytometer. The results are shown in FIG. 9. As a result, it was found that the KUM2 cells and the BMSC cells were both CD90-negative.

Regarding the occurrence of the expression of the Ly6A/E(Sca-1) antigen, an antibody reaction was carried out using an FITC-labeled anti-mouse Ly6A/E(Sca-1) antibody (PM-01164A, manufactured by Pharmingen), followed by measurement with a flow cytometer. The results are shown in FIG. 10. As a result, it was found that the KUM2 cells and the BMSC cells were both Ly6A/E(Sca-1)-positive.

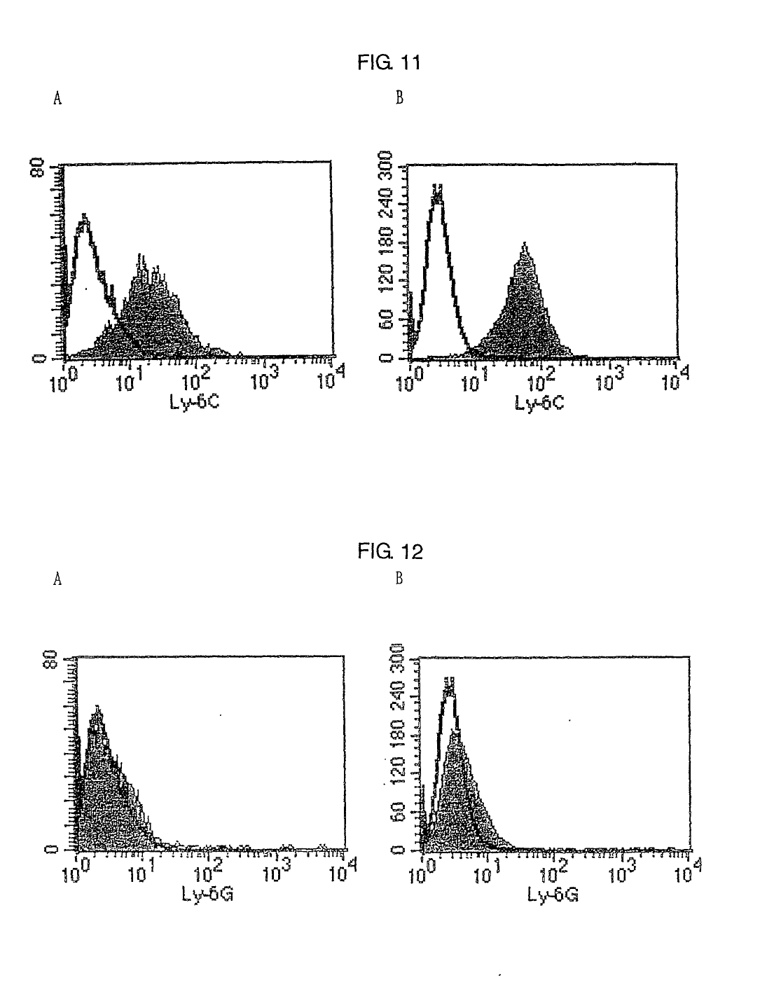

Regarding the occurrence of the expression of the Ly6c antigen, an antibody reaction was carried out using an FITC-labeled anti-mouse Ly6c antibody (PM-01152, manufactured by Pharmingen), followed by measurement with a flow cytometer. The results are shown in FIG. 11. As a result, it was found that the KUM2 cells and the BMSC cells were both Ly6c-positive.

Regarding the occurrence of the expression of the Ly6g antigen, an antibody reaction was carried out using an FITC-labeled anti-mouse Ly6g antibody (PM-01214, manufactured by Pharmingen), followed by the measurement with a flow cytometer. The results are shown in FIG. 12. As a result, it was found that the KUM2 cells and the BMSC cells were both Ly6g-negative.

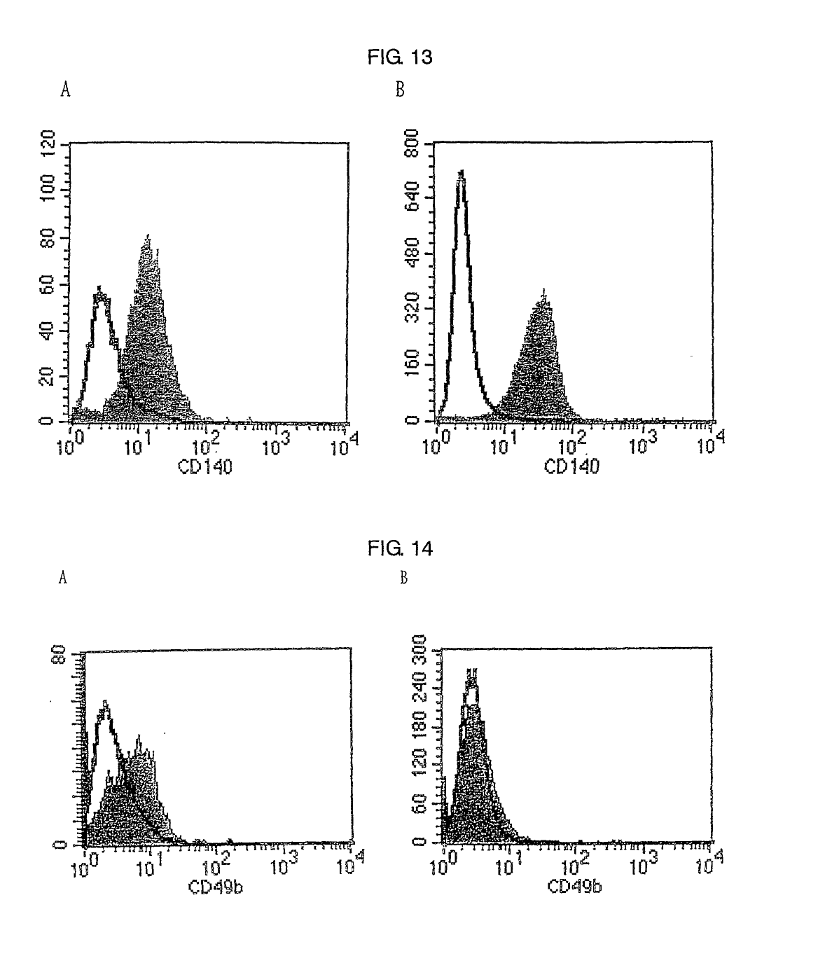

Regarding the occurrence of the expression of the CD140 antigen, an antibody reaction was carried out using a biotinylated anti-mouse CD140 antibody (PM-28011A, manufactured by Pharmingen), followed by measurement with a flow cytometer. The results are shown in FIG. 13. As a result, it was found that the KUM2 cells and the BMSC cells were both CD140-positive.

Regarding the occurrence of the expression of the CD49b antigen, an antibody reaction was carried out using an FITC-labeled anti-mouse CD49b antibody (PM-09794, manufactured by Pharmingen), followed by measurement with a flow cytometer. The results are shown in FIG. 14. As a result, it was found that the KUM2 cells were CD49b-positive, while the BMSC cells were CD49b-negative.

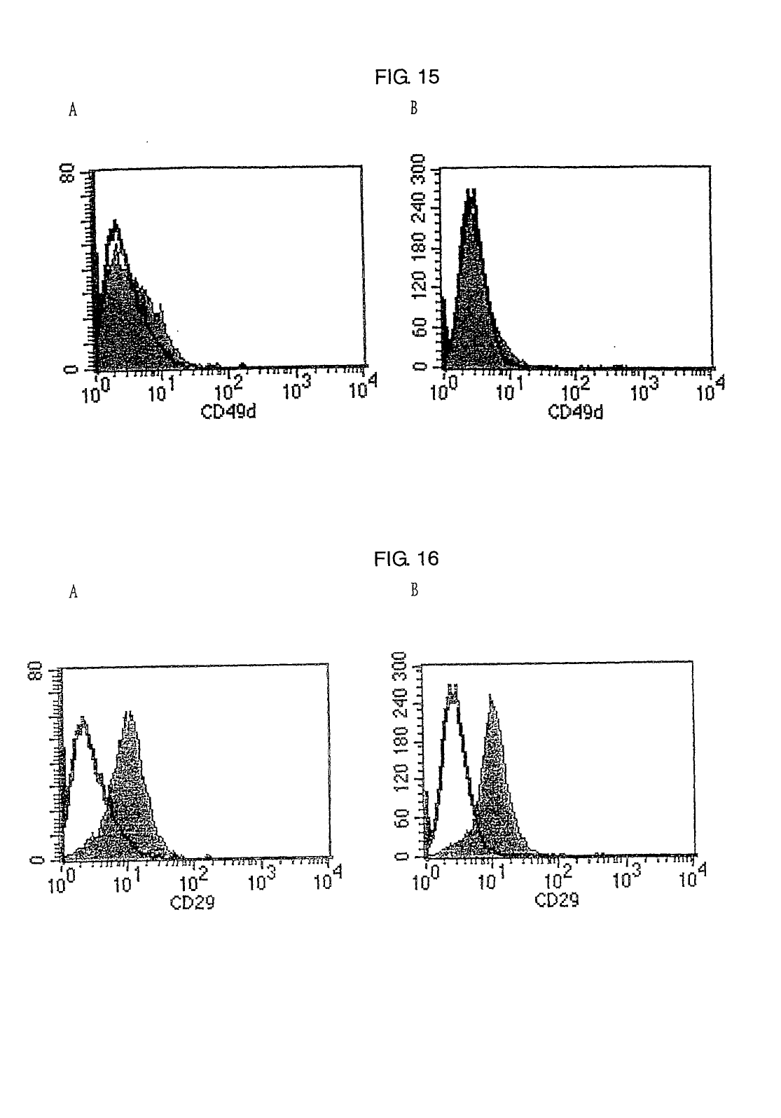

Regarding the occurrence of the expression of the CD49d antigen, an antibody reaction was carried out using an FITC-labeled anti-mouse CD49d antibody (PM-01274, manufactured by Pharmingen), followed by measurement with a flow cytometer. The results are shown in FIG. 15. As a result, it was found that the KUM2 cells and the BMSC cells were both CD49d-negative.

Regarding the occurrence of the expression of the CD29 antigen, an antibody reaction was carried out using an FITC-labeled anti-mouse CD29 antibody (PM-22634, manufactured by Pharmingen), followed by measurement with a flow cytometer. The results are shown in FIG. 16. As a result, it was found that the KUM2 cells and the BMSC cells were both CD29-positive.

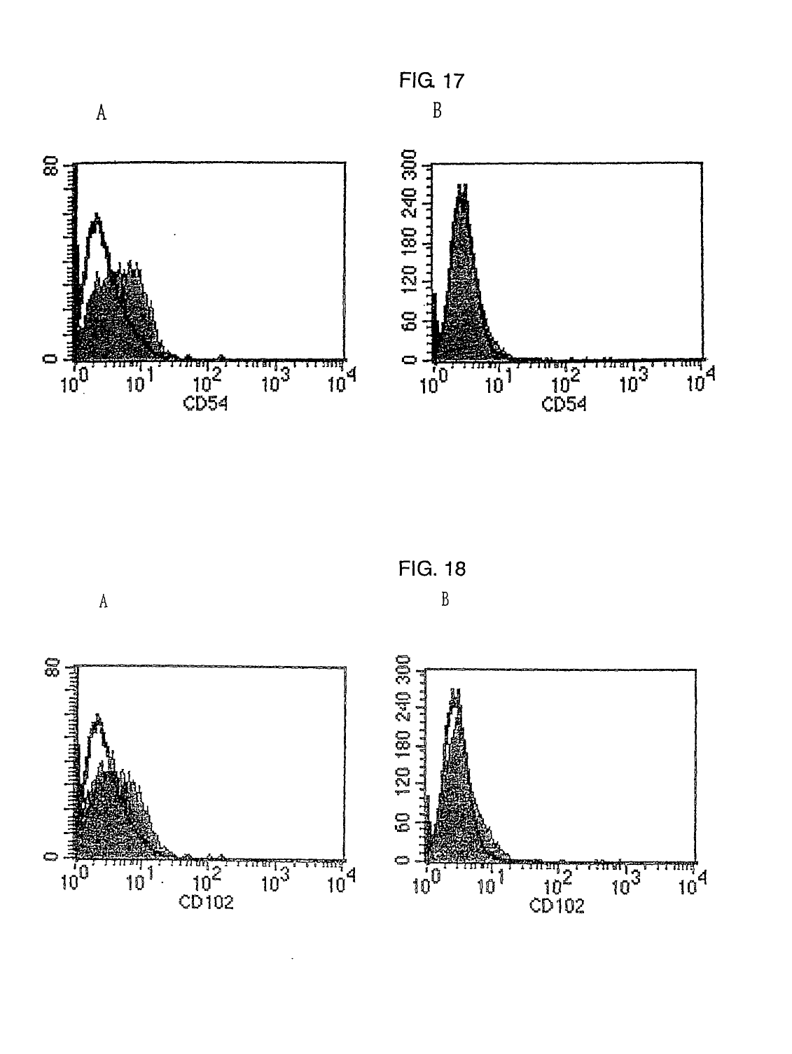

Regarding the occurrence of the expression of the CD54 antigen, an antibody reaction was carried out using an FITC-labeled anti-mouse CD54 antibody (PM-01544, manufactured by Pharmingen), followed by measurement with a flow cytometer. The results are shown in FIG. 17. As a result, it was found that the KUM2 cells were CD54-positive, while the BMSC cells were CD54-negative.

Regarding the occurrence of the expression of the CD102 antigen, an antibody reaction was carried out using an FITC-labeled anti-mouse CD102 antibody (PM-01804, manufactured by Pharmingen), followed by measurement with a flow cytometer. The results are shown in FIG. 18. As a result, it was found that the KUM2 cells and the BMSC cells were both CD102-negative.

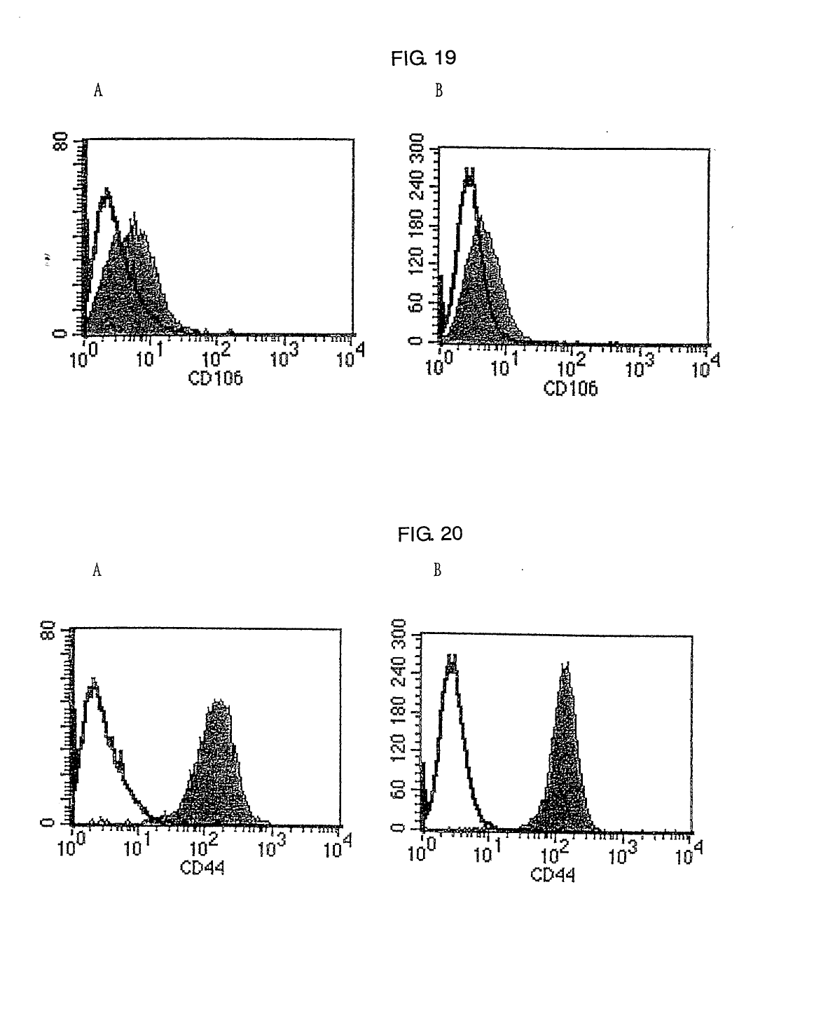

Regarding the occurrence of the expression of the CD106 antigen, an antibody reaction was carried out using an FITC-labeled anti-mouse CD106 antibody (PM-01814 manufactured by Pharmingen), followed by measurement with a flow cytometer. The results are shown in FIG. 19. As a result, it was found that the KUM2 cells were CD106-positive, while the BMSC cells were CD106-negative.

Regarding the occurrence of the expression of the CD44 antigen, an antibody reaction was carried out using an FITC-labeled anti-mouse CD44 antibody (PM-28154, manufactured by Pharmingen), followed by measurement with a flow cytometer. The results are shown in FIG. 20. As a result, it was found that the KUM2 cells and the BMSC cells were both CD44-positive.

Table 1 shows the summarized analytical data obtained using the flow cytometer.

TABLE 1

| | |
|---|---|
| KUM2 | BMSC |

Hemato

| | | |
|---|---|---|
| CD34 | − | ±*¹ |
| CD117 (c-kit) | − | + |
| CD14 | + | − |
| CD45 | − | − |
| CD90(Thyl) | − | − |
| Ly-6a/e(Scal) | + | + |
| Ly6c | + | + |
| Ly6g | − | − |

Endothelial

| | | |
|---|---|---|
| Flk-1 | − | − |
| CD31 | − | − |
| CD105 | − | − |
| CD144 | − | +*² |

Mesenchyaml

| | | |
|---|---|---|
| CD140 (PDGFR) | + | + |

Integrin

| | | |
|---|---|---|
| CD49b(α2) | + | − |
| CD49b(α4) | − | − |
| CD29(β1) | + | + |

Matrix

| | | |
|---|---|---|
| CD54(ICAM-1) | + | − |
| CD102(ICAM-2) | − | − |
| CD106(VCAM-1) | + | − |
| CD44(Hyaluronate) | + | + |

^{*1}: mixture:
^{*2}: weak positive

### EXAMPLE 11

Concentration of Differentiation Precursor Cells Using Mouse MLC2v Promoter:

In order to efficiently obtain cells having the potential to differentiate into myocardium from mouse bone marrow-derived cells having the potential to differentiate into cardiomyocytes, a promoter expression system of a mouse MLC2v (myosin light chain-2v) gene showing cardiomyocyte-specific expression was constructed. Specifically, an EGFP gene (manufactured by CLONTECH) was ligated to the downstream of the promoter sequence of the mouse MLC2v gene followed by constructing a pMLC-2-EGFP plasmid containing the expression unit of neomycin-resistance gene. DNA of this plasmid was obtained by the alkali neutralization method described in Molecular Cloning, A Laboratory Manual, 2nd ed. etc.

2 μg of the above-described DNA was introduced using LIPOFECTAMINE (manufactured by LIFE TECHNOLOGY) into KUM2 cells, which had been cultured in a 6-well plate to give 1×10⁵ cells. Detailed procedure was carried out in accordance with the manufacturer's instructions. Forty-eight hours after the gene transfection, G418 (manufactured by Sigma) was added to give a final concentration of 1 mg/ml followed by selecting survived cells which were transfected by the gene.

On the 14th day after the gene introduction, 5-aza-C was added to give a final concentration of 3 μM, and 24 hours thereafter, the medium was replaced and the differentiation was induced. From the day 3 after the induction of the differentiation, GFP-positive cells were observed. On the day 4 after the induction of the differentiation, GFP-positive cells were exclusively selected from among 1×10⁴ cells using an FACS Caliber (manufactured by Becton Dickinson) and the culturing was further continued. As a result, 90% or more cells had differentiated into cells having a myotube-like structure, which indicates that cells with differentiation potency could be efficiently concentrated. After collecting by FACS, these GFP-positive cells were transplanted in accordance with the method of Example 10. As a result, these cells differentiated not into hemoendothelium but specifically into muscle tissues such as skeletal muscle and myocardium.

### EXAMPLE 12

INDUCTION of Adipocytes from Mouse Bone Marrow-Derived Cells having the Potential to Differentiate into Cardiomyocytes:

Bone marrow cells (BMSC) having the potential to differentiate into cardiomyocytes can be induced to differentiate not only into cardiomyocytes but also into adipocytes. To control the differentiation into adipocytes, the conditions for the induction of the differentiation were examined. First, the expression of PPAR-y receptors was analyzed by the quantitative PCR method. As a result, it was found that PPAR-γ1 receptor was expressed but PPARγ2 receptor was not expressed in the BMSCs. Subsequently, PPAR-γ receptor agonists, pioglitazone and troglitazone, were added at various concentrations to bone marrow cells (BMSC) having the potential to differentiate into cardiomyocytes. As a result, the differentiation into adipocytes was promoted, depending on the concentration, and about 50% and 100% of the BMSCs differentiated into adipocytes respectively at 0.4 μM and 2 μM.

### EXAMPLE 13

Induction of Differentiation into Neurocytes, Hapatocytes and Cardiomyocytes of Mouse Bone Marrow-Derived Cells having the Potential to Differentiate into Cardiomyocytes by Transplantation into Blastocysts:

In order to obtain stable transformants of GFP-labeled bone marrow cells (BMSC) having the potential to differentiate into cardiomyocytes, gene transfection was first performed in the following manner.

GFP was introduced into a retrovirus vector plasmid pCLNCX (manufactured by Imgenex) to prepare PCLNC-GFP. The retrovirus vector plasmid PCLNC-GFP and a pCMV-Eco plasmid vector (manufactured by Imgenex) capable of expressing an ecotropic gene were treated by the alkali neutralization method and the PEG precipitation method described in Molecular Cloning, A Laboratory Manual, 2nd ed. etc. to obtain DNAs of high purity.

A day before the transfection of these DNAs, 293 cells carrying gag and pol genes, which had become confluent were subculured into a 10 cm dish by a dilution ratio of ⅕ and cultured at 37° C. in a 5% CO₂-incubator.

The transfection was carried out in the following manner.

In 0.5 ml of 250 mM CaCl₂ (pH 6.95), 5 μg of the pCLNC-GFP retrovirus vector plasmid DNA and 5 μg of the pCMV-Eco plasmid vector DNA were dissolved. The solution thus obtained was dropped into 0.5 ml of 2× BBS (50 mM BES (N,N-bis(2-hydroxyethl)-2-aminoethanesulfonci acid), 280 mM NaCl, 1.5 mM Na₂HPO₄ (pH 6.95)) in a 15 ml tube and allowed to stand at room temperature for 10 minutes. Subsequently, the DNA solution was dropped into the medium of the 293 cells prepared on the previous day and cultured at 37° C. in a 5% CO₂-incubator. On the next day, the medium was replaced and culture was further continued at 37° C. in a 5% CO₂-incubator.

Two days after the replacement of the medium, the culture supernatant was filtered through a 0.45 μm filter (manufactured by Millipore) and a solution containing the virus vector was collected.

The mouse bone marrow cells (BMSC) having the potential to differentiate into cardiomyocytes, into which the virus vector was introduced, were plated into a 6-well dish at a density of 2×10⁴ cells/well on the previous day of the infection with the virus.

Hexadimethrine bromide(polybrene) (manufactured by Sigma) was added to the virus vector-containing solution obtained above to give a final concentration of 8 μg/ml. After replacing by the medium of the mouse bone marrow cells (BMSC) having the potential to differentiate into cardiomyocytes, followed by culturing at 33° C. in a 5% CO₂-incubator. Five hours thereafter, the medium was replaced with fresh IMDM, followed by further culturing at 33° C. in a 5% CO₂-incubator.

After culturing for two days, G418 was added until the final concentration of G418 came to be 300 μg/ml, followed by further culturing for further 7 days. During this period, a part of the cells died to be suspended. The surviving cells were suspended with trypsin and scattered in a fresh culture dish.

The obtained GFP-labeled bone marrow-derived cells having the potential to differentiate into cardiomyocytes were grown in a 6-cm culture dish. After eliminating the medium, 0.5 ml of 0.25% trypsin EDTA was added and the treatment was carried out for 1 minute. Then, 1.5 ml of a fresh medium was added and the cells were suspended. After adding feral bovine serum (manufactured by Lexicon Genetics) and mixing, the cell suspension was poured into mouse blastocyst. The mouse balstocysts were obtained by spontaneously mating female C57B1/6J mice subjected to hyper-ovulation with male mice of the same line, taking out the uterus 4 days thereafter, and perfusing the inside of the uterus with M15 medium. After allowing to stand at 37° C. under 5% CO₂ until the balstocyst cavities sufficiently dilated, the balstocyst were transferred into M15 medium containing 20 mM HEPES which was cooled to about 4° C. Then, 10 to 15 BMSCs were microscopically injected into each balstocyst cavity while observing under an =inverted microscope (manufactured by Nikon) provided with a microinjector (manufactured by Narumo Kagaku) and a micromanipulator (manufactured by Narumo Kagaku). After allowing to stand at 37° C. under 5% CO₂ until the balstocyst cavities sufficiently dilated, the blastocysts were transplanted into the oviducal side of the uterus of female MCH mice with pseudopregnancy, followed by implantation. The female MCH mice with pseudopregnancy were prepared by mating with vasoligated male MCH mice aged 10 weeks or more on 17:00 three days before the transplantation at the ratio of 1:1. On 9:00 on the next morning, vaginal plugs were confirmed, and two days thereafter, the female mice were used for the above-described purpose.

The mice thus born were sacrificed and organs were extirpated for observing the expression of GFP. As a result, the expression of GFP was observed in the brain and the liver, which suggested that the BMSCs had differentiated into the nerve system and the liver. Genomic DNA was obtained from the heart taken out from another individual and subjected to PCR using the primers of SEQ ID NOS:79 and 80. As a result, it was confirmed that BMSCs were also incorporated into the heart. These results indicate that BMSCs have a totipotency of differentiating into all of the three germ layers of nerve, heart and liver.

### EXAMPLE 14

Telomerase Activity in Mouse Bone Marrow Cells having the Potential to Differentiate into Cardiomyocytes:

The mouse bone marrow cells having the potential to differentiate into cardiomyocytes were examined for telomerase activity by the Telomeric Repeat Amplification Protocol (TRAP) method (TRAPeze Telomerase Detection kit, manufactured by Oncor). The measurement of the telomerase activity was carried out as described below according to, in principle, the manufacture's instructions. The mouse bone marrow cells having the potential to differentiate into cardiomyocytes which had been cultured in a 6-cm culture dish (about 106 cells) were washed with PBS, followed by addition of 200 μl of 1× CHAPS solution. After being allowed to stand on ice for 30 minutes, the cells were recovered together with the solution to a 1.5 ml centrifuge tube and centrifuged at 14000 rpm for 20 minutes (4° C.; himac CF15, manufactured by Hitachi, Ltd.). The supernatant was recovered as a cell extract and the protein content was determined using Protein Assay (manufactured by BioRad). The protein content of the cell extract made from the mouse bone marrow cells having the potential to differentiate into cardiomyocytes under the above conditions was found to be about 1 mg/ml.

The cell extract was then subjected to telomerase elongation reaction and PCR amplification according to the manufacture s instructions. As the Taq polymerase, EX Taq polymerase (manufactured by Takara Shuzo) was used. After completion of the reactions, the samples were mixed with a 1/10 volume of 10× staining solution (0.25% bromophenol blue, 0.25% xylene cyanol FF, and 30% glycerol) and subjected to electrophoresis or 12.5% polyacrylamide gel (prepared according to the manufacture's instructions of TRAPeze Telomerase Detection Kit) at a constant voltage of 250 mV. After the electrophoresis, the gel was stained with Cyber Green (FMC) and analyzed using a fluorescence image analyzer, FluoroImager (manufactured by Molecular Dynamics). The telomerase activity was detected in the samples of the cell extracts having final concentration of 0.4-4 μg/ml.

### EXAMPLE 15

Isolation and Culturing of Bone Marrow Cell having the Potential to Differentiate into Cardiomyocyte from Rat Bone Marrow:

Six female Wistar rats of five week age (SLC Japan) were subjected to cervical dislocation and then disinfected by sufficiently applying 70% ethanol. Next, the skin of each leg was incised in a broad range and muscles covering the thighbone and shinbone were cut out to obtain the thighbone and shinbone. The thus obtained thighbone and shinbone were transferred into a culture dish of 10 cm in diameter (manufactured by Iwaki Glass) filled with PBS (manufactured by Gibco BRL) and muscles and joints were completely removed. Next, both ends of these bones were cut out using scissors, and the contents of bone marrow were squeezed out with a water flow of a culture liquid (D-PBS, manufactured by Gibco BRL) using a 10 ml syringe (manufactured by Terumo) equipped with a 2OG needle. The thus obtained cell mass was loosened into a homogeneous level by passing through the syringe. The thus obtained cell suspension was recovered into a 50 ml capacity centrifugation tube (manufactured by BECTON DICKINSON) and centrifuged at 1,500 rpm for 10 minutes (a low speed centrifuge manufactured by TOMY), and the precipitated cells were suspended in 6 ml of D-PBS. When the number of cells was counted using a modified Neubauer counting chamber, the recovered cells were 2.6×10⁹ in total. This result means that 1×10⁸ cells were recovered from one thighbone or shinbone. The thus recovered cells were diluted to a density of 1.3×10⁸ cells per 1 ml, 5 ml of the resulting suspension was overlaid on a 1.073 g/ml Percoll (manufactured by Amersham Pharmacia Biotech)/D-PBS solution (25 ml) which had been put into a 50 ml capacity centrifugation tube, followed by centrifugation at room temperature and at 3,100 rpm for 30 minutes. After the centrifugation, cells were recovered from the interface between the Percoll solution and cell suspension, diluted to 4 times with D-PBS and centrifuged at 2,300 rpm for 10 minutes and then the thus fractionated cells were recovered. The thus recovered cells were suspended in IMDM medium (manufactured by Gibco BRL) containing 20% FCS, 100 μg/ml penicillin, 250 ng/ml streptomycin and 85 μg/ml amphotericin (manufactured by Gibco BRL). When the number of cells at this stage was again counted, the recovered bone marrow cells were 4.7×10⁷ in total, meaning that cells corresponding to about 2% of the cells before the treatment were recovered. The fractionated bone marrow cells were plated on three culture dishes for animal cells having a diameter of 10 cm (manufactured by Iwaki Glass, hereinafter referred to as “10-cm culture dish”) to a density of 2 to 5×105 cells/cm² and cultured at 33° C. in a 5% CO₂-incubator (manufactured by Tabai). A half volume of the medium was exchanged with a fresh medium after 24 hours and 72 hours. Three or 4 days thereafter, a half volume of the medium was exchanged with a fresh medium. Since colonies became dense after a lapse of 15 days, the cells were removed with a trypsin EDTA treatment and a ⅔ part of them was suspended in 4 ml of a stock solution (10% DMSO, 50% bone marrow cell culture supernatant and 40% the above medium which had not been used), dispensed in 1 ml portions into 2 ml capacity tubes (manufactured by Sumitomo Bakelite) and stored in a freezer, and the remaining ⅓ part was again inoculated into two 10-cm culture dishes and subcultured.

### EXAMPLE 16

Evaluation of Rat Bone Marrow-Derived Cell having the Potential to Differentiate into Cardiomyocyte:

The rat bone marrow cells subcultured in the above were again removed with the trypsin EDTA treatment when they became dense and inoculated into a 6 well plate (manufactured by BECTON DICKINSON) in 5×10⁴ cells per well or into a 6 cm diameter culture dish coated with human fibronectin (Biocoat, manufactured by BECTON DICKINSON) in a density of 1.3×10⁵ cells. One day thereafter, culturing was carried out under two different conditions, one in which only 5-azacytidine (manufactured by Sigma, 10 μM in final concentration) was added, and another in which 5-azacytidine, PDGF-BB (manufactured by Pepro Tech EC LTD, 10 ng/ml in final concentration) and all-trans retinoic acid (RA, manufactured by Sigma, 10⁻⁹ M in final concentration) were added, and the medium was exchanged after 2 days of the culturing (in the latter conditions, PDGF and all-trans retinoic acid were again added at the time of the medium exchange and after 2 days and 4 days). Three or 4 days thereafter, the medium was exchanged, followed by culturing for 3 weeks. As a result, differentiation of myotube-like cells was observed in the conditions in which 5-azacytidine, PDGF-BB and retinoic acid were added.

### EXAMPLE 17

Forced Expression of Transcription Factor MesP1 and Enhancement of Cardiomyocyte Differentiation by Addition of Cytokine:

Influences of forced expression of a cardiomyocyte differentiation-related transcription factor MesP1 in a bone marrow-derived pluripotent stem cell (BMSC) having the potential to differentiate into cardiomyocytes upon its differentiation into cardiomyocytes and influences of a combination of forced expression of MesP1 with cytokine (FGF-8, ET-1, Midkine or BMP4) upon differentiation into cardiomyocytes were examined.

A mouse bone marrow-derived pluripotent stem cell (BMSC-MesP1) having the potential to differentiate into cardiomyocytes in which the MesP1 gene was forced-expressed was obtained using a retrovirus vector in the same manner as in Example 6, and then the differentiation was induced to examine efficiency of differentiation into cardiomyocytes.

The bone marrow cell (BMSC-MesP1) having the potential to differentiate into cardiomyocytes in which MesP1 was forced expressed was plated into a 60-mm culture dish in a density of 2×10⁴ cells/ml and cultured at 33° C. in a 5% CO₂-incubator. On the next day, 5-aza-C was added to the culture medium to give a final concentration of 3 μM, followed by culturing under five different conditions, namely (i) addition of FGF-8 to give a final concentration of 10 ng/ml (culture dish N), (ii) addition of ET-1 to give a final concentration of 10 ng/ml (culture dish P), (iii) addition of Midkine to give a final concentration of 10 ng/ml (culture dish Q), (iv) addition of BMP4 to give a final concentration of 10 ng/ml (culture dish R), and (v) no addition (culture dish S).

On the next day, the medium was exchanged with a fresh medium in order to eliminate 5-aza-C from the medium, and then the culturing was continued by adding FGF-8 to the culture dish N to give a final concentration of 10 ng/ml, ET-1 to the culture dish P to give a final concentration of 10 ng/ml, Midkine to the culture dish Q to give a final concentration of 10 ng/ml and BMP4 to the culture dish R to give a final concentration of 10 ng/ml. Two days and 4 days thereafter, the medium exchange and addition of FGF-8, ET-1, Midkine or BMP4 were carried out similarly.

Four weeks after the addition of 5-aza-C, morphology of the cells was observed under a phase contrast microscope. As a result, the number of myotube-like cells was not changed greatly by the forced expression of MesP1. In addition, about 50% of the cells became myotube-like cells in the culture dish to which FGF-8, ET-1, Midkine or BMP4 had been added.

Next, RNA was recovered from the thus obtained myotube-like cells, and genes expressing in the myotube-like cells were analyzed by quantitative PCR using the synthetic oligonucleotides shown in SEQ ID NOS:71 to 78. As a result, expression of ANP as a gene specific for a myocardium was accelerated by the forced expression of MesP1. On the other hand, FGF-8, ET-1, Midkine or BMP4 did not further accelerate the expression of ANP accelerated by the forced expression of MesP1.

Industrial Applicability

The present invention provides a bone marrow cell, a growth factor, a vitamin and an adhesion molecule which are effective for treating a heart disease accompanied with destruction and denaturation of a cardiomyocyte and for screening a therapeutic agent for it, and application methods thereof.

Free Text of Sequence Listings:

SEQ ID NO:33—Explanation of artificial sequence: Synthetic DNA

SEQ ID NO:34—Explanation of artificial sequence: Synthetic DNA

SEQ ID NO:35—Explanation of artificial sequence: Synthetic DNA

SEQ ID NO:36—Explanation of artificial sequence: Synthetic DNA

SEQ ID NO:37—Explanation of artificial sequence: Synthetic DNA

SEQ ID NO:38—Explanation of artificial sequence: Synthetic DNA

SEQ ID NO:39—Explanation of artificial sequence: Synthetic DNA

SEQ ID NO:40—Explanation of artificial sequence: Synthetic DNA

SEQ ID NO:41—Explanation of artificial sequence: Synthetic DNA

SEQ ID NO:42—Explanation of artificial sequence: Synthetic DNA

SEQ ID NO:43—Explanation of artificial sequence: Synthetic DNA

SEQ ID NO:44—Explanation of artificial sequence: Synthetic DNA

SEQ ID NO:45—Explanation of artificial sequence: Synthetic DNA

SEQ ID NO:46—Explanation of artificial sequence: Synthetic DNA

SEQ ID NO:47—Explanation of artificial sequence: Synthetic DNA

SEQ ID NO:48—Explanation of artificial sequence: Synthetic DNA

SEQ ID NO:49—Explanation of artificial sequence: Synthetic DNA

SEQ ID NO:50—Explanation of artificial sequence: Synthetic DNA

SEQ ID NO:51—Explanation of artificial sequence: Synthetic DNA

SEQ ID NO:52—Explanation of artificial sequence: Synthetic DNA

SEQ ID NO:53—Explanation of artificial sequence: Synthetic DNA

SEQ ID NO:54—Explanation of artificial sequence: Synthetic DNA

SEQ ID NO:55—Explanation of artificial sequence: Synthetic DNA

SEQ ID NO:56—Explanation of artificial sequence: Synthetic DNA

SEQ ID NO:57—Explanation of artificial sequence: Synthetic DNA

SEQ ID NO:58—Explanation of artificial sequence: Synthetic DNA

SEQ ID NO:59—Explanation of artificial sequence: Synthetic DNA

SEQ ID NO:60—Explanation of artificial sequence: Synthetic DNA

SEQ ID NO:61—Explanation of artificial sequence: Synthetic DNA

SEQ ID NO:62—Explanation of artificial sequence: Synthetic DNA

SEQ ID NO:63—Explanation of artificial sequence: Synthetic DNA

SEQ ID NO:64—Explanation of artificial sequence: Synthetic DNA

SEQ ID NO:65—Explanation of artificial sequence: Synthetic DNA

SEQ ID NO:66—Explanation of artificial sequence: Synthetic DNA

SEQ ID NO:67—Explanation of artificial sequence: Synthetic DNA

SEQ ID NO:68—Explanation of artificial sequence: Synthetic DNA

SEQ ID NO:69—Explanation of artificial sequence: Synthetic DNA

SEQ ID NO:70—Explanation of artificial sequence: Synthetic DNA

SEQ ID NO:71—Explanation of artificial sequence: Synthetic DNA

SEQ ID NO:72—Explanation of artificial sequence: Synthetic DNA

SEQ ID NO:73—Explanation of artificial sequence: Synthetic DNA

SEQ ID NO:74—Explanation of artificial sequence: Synthetic DNA

SEQ ID NO:75—Explanation of artificial sequence: Synthetic DNA

SEQ ID NO:76—Explanation of artificial sequence: Synthetic DNA

SEQ ID NO:77—Explanation of artificial sequence: Synthetic DNA

SEQ ID NO:78—Explanation of artificial sequence: Synthetic DNA

SEQ ID NO:79—Explanation of artificial sequence: Synthetic DNA

SEQ ID NO:80—Explanation of artificial sequence: Synthetic DNA

1. A cell which has been isolated from a living tissue or umbilical blood, and which has the potential to differentiate into at least a cardiomyocyte.

2. The cell according to claim 1, wherein the living tissue is bone marrow.

3. The cell according to claim 1 or 2, wherein the cell is a multipotential stem cell.

4. The cell according to any one of claims 1 to 3, wherein the cell is a multipotential stem cell which differentiates into at least a cardiomyocyte and a vascular endothelial cell.

5. The cell according to any one of claims 1 to 4, wherein the cell is a multipotential stem cell which differentiates into at least a cardiomyocyte, an adipocyte, a skeletal muscle cell, an osteoblast, and a vascular endothelial cell.

6. The cell according to any one of claims 1 to 5, wherein the cell is a multipotential stem cell which differentiates into at least a cardiomyocyte, an adipocyte, a skeletal muscle cell, an osteoblast, a vascular endothelial cell, a nervous cell, and a hepatic cell.

7. The cell according to any one of claims 1 to 3, wherein the cell is a multipotential stem cell which differentiates into any cell in adult tissues.

8. The cell according to any one of claims 1 to 7, wherein the cell is CD117-positive and CD140-positive.

9. The cell according to claim 8, wherein the cell is further CD34-positive.

10. The cell according to claim 9, wherein the cell is further CD144-positive.

11. The cell according to claim 9, wherein the cell is further CD140-negative.

12. The cell according to claim 8, wherein the cell is CD34-negative.

13. The cell according to claim 12, wherein the cell is further CD144-positive.

14. The cell according to claim 12, wherein the cell is further CD144-negative.

15. The cell according to claim 10, wherein the cell is further CD14-negative, CD45-negative, CD90-negative, Flk-1-negative, CD31-negative, CD105-negative, CD49b-negative, CD49d-negative, CD29-positive, CD54-negative, CD102-negative, CD106-negative, and CD44-positive.

16. The cell according to claim 11, wherein the cell is further CD14-negative, CD45-negative, CD90-negative, Flk-1-negative, CD31-negative, CD105-negative, CD49b-negative, CD49d-negative, CD29-positive, CD54-negative, CD102-negative, CD106-negative, and CD44-positive.

17. The cell according to claim 12, wherein the cell is further CD14-negative, CD45-negative, CD90-negative, Flk-l-negative, CD31-negative, CD105-negative, CD49b-negative, CD49d-negative, CD29-positive, CD54-negative, CD102-negative, CD106-negative, and CD44-positive.

18. The cell according to claim 13, wherein the cell is further CD14-negative, CD45-negative, CD90-negative, Flk-1-negative, CD31-negative, CD105-negative, CD49b-negative, CD49d-negative, CD29-positive, CD54-negative, CD102-negative, CD106-negative, and CD44-positive.

19. The cell according to claim 1, which does not take up Hoechst 33342.

20. A cardiomyocyte precursor which differentiates into only cardiomyocyte induced from the cell according to any one of claims 1 to 19.

21. The cell according to any one of claims 1 to 20, which has the potential to differentiate into a ventricular cardiac muscle cell.

22. The cell according to any one of claims 1 to 20, which has the potential to differentiate into a sinus node cell.

23. The cell according to any one of claims 1 to 20, wherein the vital tissue or umbilical blood is derived from a mammal.

24. The cell according to claim 23, wherein the mammal is selected from the group consisting of a mouse, a rat, a guinea pig, a hamster, a rabbit, a cat, a dog, a sheep, a swine, cattle, a goat and a human.

25. The cell according to any one of claims 1 to 8, which is mouse bone marrow-derived multipotential stem cell BMSC (FERM BP-7043).

26. The cell according to any one of claims 1 to 25, which has the potential to differentiate into a cardiomyocyte by demethylation of a chromosomal DNA of the cell.

27. The cell according to claim 26, wherein the demethylation is carried out by at least one selected from the group consisting of demethylase, 5-azacytidine, and dimethyl sulfoxide, DMSO.

28. The cell according to claim 27, wherein the demethylase comprises the amino acid sequence represented by SEQ ID NO:1.

29. The cell according to any one of claims 1 to 28, wherein the differentiation is accelerated by a factor which is expressed in a cardiogenesis region of a fetus or a factor which acts on differentiation into a cardiomyocyte in a cardiogenesis stage of a fetus.

30. The cell according to claim 29, wherein the factor which is expressed in a cardiogenesis region of a fetus or the factor which acts on differentiation into a cardiomyocyte in a cardiogenesis stage of a fetus is at least one selected from the group consisting of a cytokine, an adhesion molecule, a vitamin, a transcription factor, and an extracellular matrix.

31. The cell according to claim 30, wherein the cytokine is at least one selected from the group consisting of a platelet-derived growth factor, PDGF; a fibroblast growth factor-8, FGF-8; an endothelin 1, ET1; a midkine; and a bone morphogenetic factor, BMP-4.

32. The cell according to claim 31, wherein the PDGF, FGF-8, ET1, midkine, and BMP-4 comprise the amino acid sequence represented by SEQ ID NO:3 or 5, the amino acid sequence represented by SEQ ID NO:64, the amino acid sequence represented by SEQ ID NO:66, the amino acid sequence represented by SEQ ID NO:68, and the amino acid sequence represented by SEQ ID NO:70, respectively.

33. The cell according to claim 30, wherein the adhesion molecule is at least one selected from the group consisting of a gelatin, a laminin, a collagen, and a fibronectin.

34. The cell according to claim 30, wherein the vitamin is retinoic acid.

35. The cell according to claim 30, wherein the transcription factor is at least one selected from the group consisting of Nkx2.5/Csx, GATA4, MEF-2A, MEF-2B, MEF-2C, MEF-2D, dHAND, eHAND, TEF-1, TEF-3, TEF-5, and MesP1.

36. The cell according to claim 35, wherein the Nkx2.5/Csx, GATA4, MEF-2A, MEF-2B, MEF-2C, MEF-2D, dHAND, eHAND, TEF-1, TEF-3, TEF-5, and MesP1 comprise the amino acid sequence represented by SEQ ID NO:9, the amino acid sequence represented by SEQ ID NO:11, the amino acid sequence represented by SEQ ID NO:13, the amino acid sequence represented by SEQ ID NO:15, the amino acid sequence represented by SEQ ID NO:17, the amino acid sequence represented by SEQ ID NO:19, the amino acid sequence represented by SEQ ID NO:21, the amino acid sequence represented by SEQ ID NO:23, the amino acid sequence represented by SEQ ID NO:25, the amino acid sequence represented by SEQ ID NO:27, the amino acid sequence represented by SEQ ID NO:29, and the amino acid sequence represented by SEQ ID NO:62, respectively.

37. The cell according to claim 30, wherein the extracellular matrix is an extracellular matrix derived from a cardiomyocyte.

38. The cell according to any one of claims 1 to 28, wherein the differentiation is inhibited by a fibroblast growth factor-2, FGF-2.

39. The cell according to claim 38, wherein the FGF-2 comprises the amino acid sequence represented by SEQ ID NO:7 or 8.

40. The cell according to any one of claims 1 to 28, which is capable of differentiating into a cardiomyocyte or a blood vessel by transplantation into a heart.

41. The cell according to any one of claims 1 to 19, which is capable of differentiating into a cardiac muscle by transplantation into a blastocyst or by co-culturing with a cardiomyocyte.

42. The cell according to any one of claims 1 to 28, which is capable of differentiating into an adipocyte by an activator of a nuclear receptor, PPAR-Y.

43. The cell according to claim 42, wherein the activator is a compound having a thiazolidione skeleton.

44. The cell according to claim 43, wherein the compound is at least one selected from the group consisting of troglitazone, pioglitazone, and rosiglitazone.

45. The cell according to any one of claims 1 to 28, which is capable of differentiating into a nervous cell by transplantation into a blastocyst or by transplantation into an encephalon or a spinal cord.

46. The cell according to any one of claims 1 to 28, which is capable of differentiating into a hepatic cell by transplantation into a blastocyst or by transplantation into a liver.

47. A method for differentiating the cell according to any one of claims 1 to 28 into a cardiac muscle, comprising using a chromosomal DNA-dimethylating agent.

48. A method for redifferentiating the cell according to claim 9 into the cell according to 12, comprising using a chromosomal DNA-dimethylating agent.

49. A method for redifferentiating a cell which is CD117-negative and CD140-positive into the cell according to claim 8, comprising using a chromosomal DNA-dimethylating agent.

50. The method according to claim 48 or 49, wherein the chromosomal DNA-dimethylating agent is selected from the group consisting of a demethylase, 5-azacytidine, and DMSO.

51. The method according to claim 50, wherein the demethylase comprises the amino acid sequence represented by SEQ ID NO:1.

52. A method for differentiating the cell according to any one of claims 1 to 28 into a cardiac muscle, comprising using a factor which is expressed in a cardiogenesis region of a fetus or a factor which acts on differentiation into a cardiomyocyte in a cardiogenesis stage of a fetus.

53. The method according to claim 52, wherein the factor which is expressed in a cardiogenesis region of a fetus or the factor which acts on differentiation into a cardiomyocyte in a cardiogenesis stage of a fetus is at least one selected from the group consisting of a cytokine, an adhesion molecule, a vitamin, a transcription factor, and an extracellular matrix.

54. The method according to claim 53, wherein the cytokine is at least one selected from the group consisting of a platelet-derived growth factor, PDGF; a fibroblast growth factor-8, FGF-8; an endothelin 1, ET1; a midkine; and a bone morphogenetic factor, BMP-4.

55. The method according to claim 54, wherein the PDGF, FGF-8, ET1, midkine, and BMP-4 comprise the amino acid sequence represented by SEQ ID NO:3 or 5, the amino acid sequence represented by SEQ ID NO:64, the amino acid sequence represented by SEQ ID NO:66, the amino acid sequence represented by SEQ ID NO:68, and the amino acid sequence represented by SEQ ID NO:70, respectively.

56. The method according to claim 53, wherein the adhesion molecule is at least one selected from the group consisting of a gelatin, a laminin, a collagen, and a fibronectin.

57. The method according to claim 53, wherein the vitamin is retinoic acid.

58. The method according to claim 53, wherein the transcription factor is at least one selected from the group consisting of Nkx2.5/Csx, GATA4, MEF-2A, MEF-2B, MEF-2C, MEF-2D, dHAND, eHAND, TEF-1, TEF-3, TEF-5, and MesP1.

59. The method according to claim 58, wherein the Nkx2.5/Csx, GATA4, MEF-2A, MEF-2B, MEF-2C, MEF-2D, dHAND, eHAND, TEF-1, TEF-3, TEF-5, and MesP1 comprise the amino acid sequence represented by SEQ ID NO:9, the amino acid sequence represented by SEQ ID NO:11, the amino acid sequence represented by SEQ ID NO:13, the amino acid sequence represented by SEQ ID NO:15, the amino acid sequence represented by SEQ ID NO:17, the amino acid sequence represented by SEQ ID NO:19, the amino acid sequence represented by SEQ ID NO:21, the amino acid sequence represented by SEQ ID NO:23, the amino acid sequence represented by SEQ ID NO:25, the amino acid sequence represented by SEQ ID NO:27, the amino acid sequence represented by SEQ ID NO:29, the amino acid sequence represented by SEQ ID NO:62, respectively.

60. The method according to claim 53, wherein the extracellular matrix is an extracellular matrix derived from a cardiomyocyte.

61. A method for differentiating the cell according to any one of claims 1 to 28 into an adipocyte, comprising using an activator of a nuclear receptor, PPAR-γ.

62. The method according to claim 61, wherein the activator is a compound having a thiazolidione skeleton.

63. The method according to claim 62, wherein the compound is at least one selected from the group consisting of troglitazone, pioglitazone, and rosiglitazone.

64. A myocardium-forming agent, comprising, as an active ingredient, a chromosomal DNA-demethylating agent.

65. The myocardium-forming agent according to claim 64, wherein the chromosomal DNA-demethylating agent is at least one selected from the group consisting of a demethylase, 5-azacytidine, and DMSO.

66. The myocardium-forming agent according to claim 65, wherein the demethylase comprises the amino acid sequence represented by SEQ ID NO:1.

67. A myocardium-forming agent, comprising, as an active ingredient, a factor which is expressed in a cardiogenesis region of a fetus or a factor which acts on differentiation into a cardiomyocyte in a cardiogenesis stage of a fetus.

68. The myocardium-forming agent according to claim 67, wherein the factor which is expressed in a cardiogenesis region of a fetus or the factor which acts on differentiation into a cardiomyocyte in a cardiogenesis stage of a fetus is at least one selected from the group consisting of a cytokine, an adhesion molecule, a vitamin, a transcription factor, and an extracellular matrix.

69. The myocardium-forming agent according to claim 68, wherein the cytokine is at least one selected from the group consisting of a platelet-derived growth factor, PDGF; a fibroblast growth factor-8, FGF-8; an endothelin 1, ETI; a midkine; and a bone morphogenetic factor, BMP-4.

70. The myocardium-forming agent according to claim 69, wherein the PDGF, FGF-8, ET1, midkine, and BMP-4 comprise the amino acid sequence represented by SEQ ID NO:3 or 5, the amino acid sequence represented by SEQ ID NO:64, the amino acid sequence represented by SEQ ID NO: 66, the amino acid sequence represented by SEQ ID NO:68, and the amino acid sequence represented by SEQ ID NO:70, respectively.

71. The myocardium-forming agent according to claim 68, wherein the adhesion molecule is selected from the group consisting of a gelatin, a laminin, a collagen, and a fibronectin.

72. The myocardium-forming agent according to claim 71, wherein the vitamin is retinoic acid.

73. The myocardium-forming agent according to claim 68, wherein the transcription factor is at least one selected from the group consisting of Nkx2.5/Csx, GATA4, MEF-2A, MEF-2B, MEF-2C, MEF-2D, dHAND, eHAND, TEF-1, TEF-3, TEF-5, and MesP1.

74. The myocardium-forming agent according to claim 73, wherein the Nkx2.5/Csx, GATA4, MEF-2A, MEF-2B, MEF-2C, MEF-2D, dHAND, eHAND, TEF-1, TEF-3, TEF-5, and MesP1 comprise the amino acid sequence represented by SEQ ID NO:9, the amino acid sequence represented by SEQ ID NO:11, the amino acid sequence represented by SEQ ID NO:13, the amino acid sequence represented by SEQ ID NO:15, the amino acid sequence represented by SEQ ID NO:17, the amino acid sequence represented by SEQ ID NO:19, the amino acid sequence represented by SEQ ID NO:21, the amino acid sequence represented by SEQ ID NO:23, the amino acid sequence represented by SEQ ID NO:25, the amino acid sequence represented by SEQ ID NO:27, the amino acid sequence represented by SEQ ID NO:29, and the amino acid sequence represented by SEQ ID NO:62, respectively.

75. The myocardium-forming agent according to claim 68, wherein the extracellular matrix is an extracellular matrix derived from a cardiomyocyte.

76. A method for regenerating a heart damaged by a heart disease, comprising using the cell according to any one of claims 1 to 46.

77. An agent for cardiac regeneration, comprising, as an active ingredient, the cell according to any one of claims 1 to 46.

78. A method for specifically transfecting a wild-type gene corresponding to a mutant gene in a congenital genetic disease to a myocardium, comprising using the cell according to any one of claims 1 to 46 into which the wild-type gene corresponding to a mutant gene in a congenital genetic disease of a heart has been introduced.

79. A therapeutic agent for a heart disease, comprising, as an active ingredient, the cell according to any one of claims 1 to 46 into which a wild-type gene corresponding to a mutant gene in a congenital genetic disease of a heart has been introduced.

80. A method for producing an antibody which specifically recognizes the cell according to any one of claims 1 to 46, comprising using the cell as an antigen.

81. A method for isolating a cell having the potential to differentiate into a cardiomyocyte according to any one of claims 1 to 46, comprising using an antibody obtained by the method according to claim 80.

82. A method for obtaining a surface antigen specific for the cell according to any one of claims 1 to 46, comprising using the cell.

83. A method for screening a factor which proliferates the cell according to any one of claims 1 to 46, comprising using the cell.

84. A method for screening a factor which induces the cell according to any one of claims 1 to 46 to differentiate into a cardiomyocyte, comprising using the cell.

85. A method for screening a factor which immortalizes the cell according to any one of claims 1 to 46, comprising using the cell.

86. A method for immortalizing the cell according to any one of claims 1 to 46, comprising expressing a telomerase in the cell.

87. The method according to claim 86, wherein the telomerase comprises the amino acid sequence represented by SEQ ID NO:31.

88. A therapeutic agent for a heart disease, comprising, as an active ingredient, the cell according to any one of claims 1 to 46 which has been immortalized by expressing a telomerase.

89. The therapeutic agent according to claim 88, wherein the telomerase comprises the amino acid sequence represented by SEQ ID NO:31.

90. A culture supernatant comprising the cell according to any one of claims 1 to 46.

91. A method for inducing the cell according to any one of claims 1 to 46 to differentiate into a cardiomyocyte, comprising using the culture supernatant according to claim 90.
